(19) United States
(12) Patent Application Publication
(10) Pub. No.: US 2016/0357901 A1
(43) Pub. Date: Dec. 8, 2016

(54) PEPTIDOMIMETIC COMPOUNDS AND RELATED METHODS

(71) Applicant: Kevin Burgess, College Station, TX (US)

(72) Inventor: Kevin Burgess, College Station, TX (US)

(21) Appl. No.: 15/089,828

(22) Filed: Apr. 4, 2016

Related U.S. Application Data
(63) Continuation of application No. 13/866,277, filed on Apr. 19, 2013, now abandoned.
(60) Provisional application No. 61/636,138, filed on Apr. 20, 2012.

(51) Int. Cl. G06F 19/12; C12Q 1/48; C12Q 1/37

(52) U.S. Cl. CPC G06F 19/12; C12Q 1/37; C12Q 1/48; G01N 2333/8139; G01N 2333/91028; G01N 2333/8142; G01N 2333/90203

(57) ABSTRACT

Provided herein are compounds and methods of using same for the perturbation and/or inhibition of protein-protein interactions. Also provided herein is a data mining method useful for the identification of protein-protein interactions that may be inhibited by these compounds.

### RELATED APPLICATIONS

This application claims priority to U.S. Provisional Application No. 61/636,138, filed Apr. 20, 2012, the entire contents of which are incorporated herein by reference.

### BACKGROUND

Methods to facilitate discovery of small molecules that perturb protein-protein interactions (PPIs) include high throughput screening (HTS), fragment- and structure-based strategies, molecular evolution of macrocycles, and design of secondary structure mimics. However, even the most prevalent method, HTS, gives disappointing hit-rates relative to the cost and time expenditures involved. Computational simulations based on matching virtual libraries with computed physiochemical parameters may be used to augment HTS, but rarely replace it for PPI targets.

Compound collections for HTS are typically assembled to find small molecules that bind enzyme active sites, ion channels, and G-coupled protein receptors, based on predicted oral bioavailabilities. It has been suggested that hit-rates in HTS against PPI targets are disappointing because the compound collections do not have appropriate chemotypes. Despite this, there is no widely accepted notion of the ideal types of small molecules.

Accordingly, there remains a need to identify small molecules that perturb or inhibit protein-protein interactions (PPIs).

### SUMMARY

Described herein is a method with the potential to solve the problem of designing small molecule probes to perturb PPIs. To do this we have defined suitable chemotypes, established an approach to elucidate the intrinsically preferred conformations of the small molecule scaffolds, and devised an algorithm to mine huge databases of structurally characterized PPIs to find ones that match these conformations well. We also developed a synthesis of a good small molecule scaffold, applied the algorithm to this, and proved it can be used to design molecules to interfere with a PPI (the dimerization region of HIV-1 protease).

At a minimum, the concept described here will establish an idea generating method to inspire the design of small molecules to perturb PPIs. At best, the proposed method could provide a time and cost-effective alternative to HTS in the pharmaceutical industry and in academia.

With regards to ideal small molecule chemotypes to interfere with PPIs, we agree with others who have argued that expression of amino acid side-chains on semi-rigid small molecules is a valuable concept because interactions between interface side-chains dominate PPIs. Molecules of these types are often designed to resemble secondary structures of protein components at PPI interfaces. However, secondary structure mimicry is limited because PPI hot-spots are often formed from more than one, or from non-ideal, structural motifs. These observations led us to conclude that semi-rigid small molecules are suitable chemotypes, but their design should be primarily based on comparing the orientations of the amino acid side-chains they project with those at protein-protein interfaces, rather than secondary structures.

The level of detail about side-chain orientations that is required to implement the idea outlined above necessitates computational methods that can process huge datasets of structurally characterized PPIs. Consequently, we introduce a concept, Exploring Key Orientations (EKO), to pair preferred conformations of a semi-rigid small molecule with the PPI-interfaces. EKO achieves this by comparing amino acid side-chain orientations in their preferred conformations with those at protein interfaces on a massive scale via computer-assisted data mining techniques.

A major limitation to the design of secondary structure mimics has been to generate structures that are selective for specific PPIs. A key innovation described here is to use data-mining for the reverse process: to find PPIs that match preferred small molecule conformations of the featured interface mimic. The EKO approach achieves this matching process irrespective of whether the small molecule resembles a secondary structure or not. Similarly, the featured concept is the inverse of the HTS where an assay is selected for a particular PPI and huge libraries are screened against it; EKO is chemistry-driven, whereas HTS is target-driven. As far as we are aware, EKO is the first data-mining approach to match PPIs with probes via virtual affinity selection from a huge PPI library using specific small molecule baits.

Accordingly, in one aspect, provided herein is a compound of formula (I):

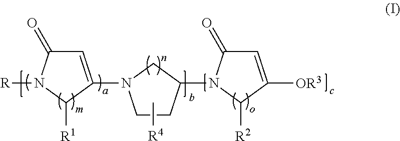

wherein R is selected from hydrogen, alkyl, heteroalkyl, or a nitrogen protecting group;
each R¹ and R² is independently selected from hydrogen, alkyl, cycloalkyl, heteroalkyl, hetercycloalkyl, aryl, heteroaryl, alkyl-aryl, alkyl-heteroaryl and alkyl-heterocycloalkyl, wherein each R¹ is optionally, independently substituted one or more times with substituents selected from oxo, carboxyl (i.e., —CO₂H), carboxamide, carboxyalkyl, hydroxyl, alkoxy, amino, aminoalkyl, thio, thioalkyl and seleno);
R³ is selected from hydrogen, alkyl, heteroalkyl or an oxygen protecting group;
R⁴ is selected from hydrogen, alkyl, alkoxy, aryl and heteroaryl;
Each m is independently 1-2;
Each n is independently 0-2;
Each o is independently 1-2; a is 0-1;
b is 1-3; and
c is 0-1;
wherein, when b is greater than 1, each R¹ is independently selected from hydrogen, alkyl, cycloalkyl, heteroalkyl, hetercycloalkyl, aryl, heteroaryl, alkyl-aryl, alkyl-heteroaryl and alkyl-heterocycloalkyl, each R⁴ is independently selected from hydrogen, alkyl, alkoxy, aryl and heteroaryl and each n is independently 0-2.

In one embodiment of formula (I), each m, n and o is 1. In another embodiment of formula (I), each m, n and o is 2. In still another embodiment, each m and o is 1, and each n is 2.

In one embodiment of formula (I), R¹ and R² are independently selected from the side chains of naturally occurring amino acids, and enantiomers thereof.

In another embodiment, the compound of formula (I) is a compound having the structure of formula (II):

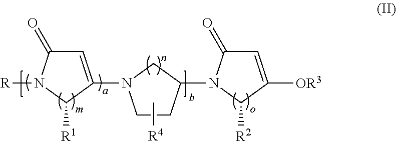

wherein R, R¹-R⁴, a, b, m, n and o are as defined above.

In another embodiment, the compound of formula (I) is a compound having the structure of formula (III):

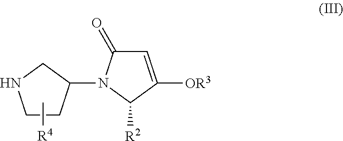

wherein R²-R⁴ are as defined above.

In one embodiment of formula (III), R⁴ is H and R² is selected from methyl, sec-butyl and benzyl.

In another embodiment, the compound of formula (I) is a compound having the structure of formula (IV):

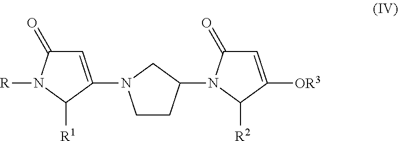

wherein R, R¹-R³ are as defined above.

In one embodiment of formula (IV), R is hydrogen; R³ is ^{t}Bu; and R¹ is methyl and R² is methyl; or R¹ is methyl and R² is benzyl; or R¹ is iso-butyl and R² is methyl; or R¹ is —CH₂CH₂SCH₃ and R² is sec-butyl; or R¹ is benzyl and R² is benzyl; or R¹ is —CH(OBn)CH₃ and R² is sec-butyl; or R¹ is CH₂C(O)OBn and R² is sec-butyl.

In another embodiment, the compound of formula (I) is a compound having the structure of formula (V):

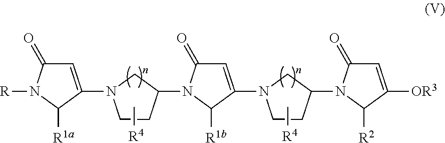

wherein R^{1a}, R^{1b} and R² are independently selected from hydrogen, alkyl, cycloalkyl, heteroalkyl, hetercycloalkyl, aryl, heteroaryl, alkyl-aryl, alkyl-heteroaryl and alkyl-heterocycloalkyl, each R⁴ is independently selected from hydrogen, alkyl, alkoxy, aryl and heteroaryl and each n is independently 0-2.

In another embodiment, the compound of formula (V) is a compound having the structure of formula (VI):

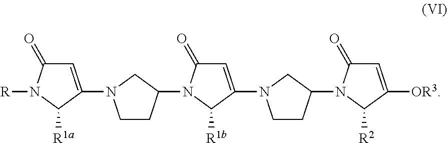

In one embodiment of formula (VI). A compound of claim 9 wherein R is hydrogen, R³ is ^{t}Bu and wherein: R^{1a}, R^{1b} and R² are each methyl; or R^{1a} is iso-butyl, R^{1b} is methyl and R² is sec-butyl.

In another embodiment, the compound of formula (I) is a compound having the structure of formula (VII):

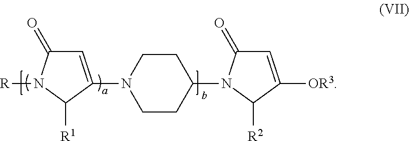

In another embodiment, the compound of formula (VII) is a compound having the structure of formula (VIII):

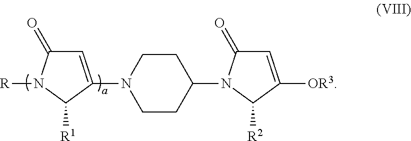

In one embodiment of the compound of formula (VIII), R is hydrogen; a is 0; R³ is ^{t}Bu; and R² is selected from methyl, benzyl, —CH₂-(3)-indoyl, —CH₂O^{t}Bu and —(CH₂)₄NH₂.

In another embodiment formula (VIII), R is hydrogen; a is 1; R³ is ^{t}Bu; and R¹ is methyl and R² is methyl; or R¹ is methyl and R² is benzyl; or R¹ is methyl and R² is benzyl; or R¹ is methyl and R² is —CH₂-(3)-indoyl; or R¹ is methyl and R² is —CH₂O^{t}Bu; or R¹ is methyl and R² is —(CH₂)₄NH₂; or R¹ is benzyl and R² is methyl; or R¹ is benzyl and R² is —CH₂O^{t}Bu; or R¹ is —(CH₂)₂SCH₃ and R² is methyl.

In yet another embodiment, the compound of formula (VIII) is a compound having the structure of formula (IX):

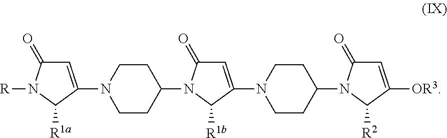

In one embodiment of the compound of formula (IX), R is hydrogen; R^{1a}, R^{1b} and R² are each methyl; and R³ is O^{t}Bu.

In another aspect, provided herein is a method for inhibiting protein-protein interactions, comprising contacting the interacting proteins with a compound of formula (I). In one embodiment, the compound of formula (I) has a structure according to any one of formulas (V), (VI) or (IX). In another embodiment, the protein-protein interaction is a dimerization. In still another embodiment, the protein is HIV-1 protease.

In another aspect, provided herein is a method for identifying protein-protein interactions, comprising:
(i) calculating the preferred conformation(s) of a compound;
(ii) characterizing the preferred conformation(s) in terms of the coordinates of the Cα and Cβ atoms of the side chains of the compound;
(iii) searching structural databases for protein-protein interactions wherein the orientations of amino acid side chains at the protein-protein interface match the Cα and Cβ coordinates of the preferred conformation(s).

In one embodiment of the method for identifying protein-protein interactions, the compound is the compound of formula (I). In yet another aspect, provided herein is a method for identifying protein-protein interactions that contain an interface region where the orientations of three amino acid side-chains match the Cα and Cβ coordinates of one or more preferred conformations of a compound, comprising:
(i) calculating the preferred conformation(s) of the compound;
(ii) characterizing the preferred conformation(s) in terms of the coordinates of the Cα and Cβ atoms of the side chains of the compound;
(iii) searching structural databases for protein-protein interactions that contain an interface region where the orientations of three amino acid side-chains match the Cα and Cβ coordinates of one or more preferred conformations of the compound.

In yet another aspect, provided herein is a method for identifying protein-protein interactions, comprising searching structural databases for protein-protein interactions wherein the orientations of amino acid side chains at the protein-protein interface match the Cα and Cβ coordinates of the preferred conformation(s) of a compound.

In still another aspect, provided herein is a method for identifying protein-protein interactions, comprising searching structural databases for protein-protein interactions that contain an interface region where the orientations of three amino acid side-chains match the Cα and Cβ coordinates of one or more preferred conformations of a compound.

In one embodiment of the method for identifying protein-protein interactions, the compound is the compound of formula (I).

In another aspect, provided herein is a method for selecting protein-protein interactions that may be perturbed by a molecule having two or more amino acid side-chains, comprising the steps of:
(i) simulating one or more conformations of the molecule that have energies within 3 kcal/mol of the most stable conformer located in this simulation procedure.
(ii) assigning three-dimensional coordinates to the Cα and Cβ atoms of the amino acid side chains in each conformation of (i);
(iii) assigning three-dimensional coordinates to the Cα and Cβ atoms of the amino acid side chains in each member of a group of structurally characterized protein-protein interactions;
(iv) overlaying the coordinates from (ii) on the coordinates from (iii) and measuring goodness-of-fit for each overlay
(v) selecting those overlays from (iv) having a goodness-of-fit within a predetermined tolerance.

In one embodiment of the method, the predetermined tolerance is RMSD<0.7 Å. In another embodiment, the predetermined tolerance is RMSD 0.2-0.5. In another embodiment, a computer algorithm is used for steps (i), (ii), (iii), (iv), and/or (v). In another embodiment, the molecule has two or three amino acid side chains. In another embodiment, the amino acid side chains of the molecule are each methyl. In another embodiment, the molecule has three amino acid side chains and wherein the amino acid side chains are each methyl.

In another embodiment of the method, part (iv) comprises:

selecting a protein-protein interaction wherein three sets of coordinates from part (iii) correspond within a predetermined tolerance to three sets of coordinates from part (ii).

In another embodiment of the method, one or more sets of coordinates are assigned to each one of a group of protein-protein interactions using data selected from the group consisting of crystallographic data and/or NMR data.

In another embodiment of the method, the molecule expressing one or more amino acid side-chains is a derivative of compound 1 disclosed herein.

In another embodiment of the method, the molecule expressing one or more amino acid side-chains is a compound of any one of formulas (I)-(IX).

In another embodiment of the method, protein-protein interactions that may tend to be perturbed by a given small molecule are selected that by searching structural databases of NMR and/or X-ray data for protein-protein interactions, for situations wherein the orientations of amino acid side chains at the protein-protein interface match the Cα and Cβ coordinates of amino acid side-chains expressed on the preferred conformation(s) of the small molecule.

In another aspect, provided herein is an algorithm for matching side-chain orientations in protein-protein interactions, as shown by X-ray or NMR studies, with one or more preferred conformations of a compound that has similar side chains. In one embodiment of the algorithm, the protein-protein interactions contain an interface region having at least three amino acid side-chains. In another embodiment, the orientations of three amino acid side-chains in an interface region of the protein-protein interaction is matched to the Cα and Cβ coordinates of one or more preferred conformations of a compound that also has three substituents with Cα and Cβ atoms relative to a semi-rigid organic scaffold.

In another aspect, provided herein is a computer program for instructing a computer to perform a method described herein. In one embodiment, the computer program utilizes an algorithm described herein.

### BRIEF DESCRIPTION OF THE DRAWINGS

FIG. 1. Determination of the IC₅₀ value for inhibition of HIV-1 protease.

FIG. 2. Zhang-Poorman analyses

FIG. 3. IC₅₀ determination for LAI-O^{t}Bu

FIG. 4. IC₅₀ determination for LAI-OH

FIG. 5. IC₅₀ determination for FLA-O^{t}Bu

FIG. 6. IC₅₀ determination for FLA-OH

FIG. 7. Flow chart for implementation of EKO.

FIG. 8. ICL of the protease (throughout, score is our in-house scoring function where lower is better, and ΔE is the energy of the preferred conformation over the global minimum before interaction with the protein; an alternative to RMSD). c First of two matches found for L,L,L-1 conformations by relaxing the RMSD limitation; this is on a C-terminal region slightly shifted from the original match. d As for c except this match was found for an N-terminal region. e Best overlay identified for stereoisomers of 1 gives match with the same C-terminal region as the original hit in b. f Inverse polarities of L,L,L-1 with respect to the HIV-1 protease sequence for Ile93, Cys95, Leu97; Cys95, Leu97, Phe99; and Pro1, Ile3, Leu5. It is convenient to mimic Cys side-chains with Ala (and this is justifiable based on known mutations, see text). Ala is also used as a mimic for C-terminal Pro since it is structurally impossible to make a tetramic acid analog of this in the same as for other amino acids. g Mining other isomers of 1 revealed that only D,L,L-1 preferred conformations overlaid on HIV-1 protease.

### DETAILED DESCRIPTION

As used herein, the term “alkyl” refers to a fully saturated branched or unbranched hydrocarbon moiety. Preferably the alkyl comprises 1 to 20 carbon atoms, more preferably 1 to 16 carbon atoms, 1 to 10 carbon atoms, 1 to 7 carbon atoms, 1 to 6 carbons, 1 to 4 carbons, or 1 to 3 carbon atoms. In a preferred embodiment, the alkyl contains 1 to 6 carbons. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl and the like. Furthermore, the expression “Cₓ-C_{y}-alkyl”, wherein x is 1-5 and y is 2-10 indicates a particular alkyl group (straight- or branched-chain) of a particular range of carbons. For example, the expression C₁-C₄-alkyl includes, but is not limited to, methyl, ethyl, propyl, butyl, isopropyl, tert-butyl and isobutyl.

The term “cycloalkyl”, as used herein, refers specifically to groups having three to seven, preferably three to ten carbon atoms. Suitable cycloalkyls include, but are not limited to cyclopropyl, cyclobutyl, cyclopentyl, cyclohexyl, cycloheptyl and the like, which, as in the case of aliphatic, heteroaliphatic or heterocyclic moieties, may optionally be substituted with substituents including, but not limited to aliphatic; heteroaliphatic; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CH₂OH; —CH₂CH₂OH; CH₂NH₂; —CH₂SO₂CH₃; —C(O)R_{X}; —CO₂(R_{X}); —CON(R_{X})₂; —OC(O)R_{X}; —OCO₂R_{X}; OCON(R_{X})₂—N(R_{X})₂; —S(O)₂R_{X}; —NR_{X}(CO)R_{X} wherein each occurrence of R_{X} independently includes, but is not limited to, aliphatic, heteroaliphatic, aryl, heteroaryl, alkylaryl, or alkylheteroaryl, wherein any of the aliphatic, heteroaliphatic, alkylaryl, or alkylheteroaryl substituents described above and herein may be substituted or unsubstituted, branched or unbranched, cyclic or acyclic, and wherein any of the aryl or heteroaryl substituents described above and herein may be substituted or unsubstituted.

The term “heteroalkyl”, as used herein, refers to alkyl moieties in which one or more carbon atoms in the main chain have been substituted with a heteroatom. Thus, a heteroalkyl group refers to an alkyl chain which contains one or more oxygen, sulfur, nitrogen, phosphorus or silicon atoms, e.g., in place of carbon atoms. Heteroalkyl moieties may be branched or linear unbranched. In certain embodiments, heteroalkyl moieties are substituted by independent replacement of one or more of the hydrogen atoms thereon with one or more moieties including, but not limited to aliphatic; alicyclic; heteroaliphatic; heteroalicyclic; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CH₂OH; —CH₂CH₂OH; CH₂NH₂; —CH₂SO₂CH₃; —C(O)R_{X}; —CO₂(R_{X}); —CON(R_{X})₂; —OC(O)R_{X}; —OCO₂R_{X}; OCON(R_{X})₂—N(R_{X})₂; —S(O)₂R_{X}; —NR_{X}(CO)R_{X} wherein each occurrence of Rx independently includes, but is not limited to, aliphatic, alicyclic, heteroaliphatic, heteroalicyclic, aryl, heteroaryl, alkylaryl, or alkylheteroaryl, wherein any of the aliphatic, alicyclic, heteroaliphatic, heteroalicyclic, alkylaryl, or alkylheteroaryl substituents described above and herein may be substituted or unsubstituted, branched or unbranched, cyclic or acyclic, and wherein any of the aryl or heteroaryl substituents described above and herein may be substituted or unsubstituted.

The terms “halo” and “halogen” as used herein refer to an atom selected from fluorine, chlorine, bromine and iodine.

The term “haloalkyl” denotes an alkyl group, as de fined above, having one, two, or three halogen atoms attached thereto and is exemplified by such groups as chloromethyl, bromoethyl, trifluoromethyl, and the like.

The term “aryl” includes aromatic monocyclic or multicyclic e.g., tricyclic, bicyclic, hydrocarbon ring systems consisting only of hydrogen and carbon and containing from six to nineteen carbon atoms, or six to ten carbon atoms, where the ring systems may be partially saturated. Aryl groups include, but are not limited to, groups such as phenyl, tolyl, xylyl, anthryl, naphthyl and phenanthryl. Aryl groups can also be fused or bridged with alicyclic or heterocyclic rings which are not aromatic so as to form a polycycle (e.g., tetralin).

The term “aryloxy” refers to a moiety comprising an oxygen atom that is substituted with an aryl group, as defined above.

The term “heteroaryl,” as used herein, represents a stable monocyclic or bicyclic ring of up to 7 atoms in each ring, wherein at least one ring is aromatic and contains from 1 to 4 heteroatoms selected from the group consisting of O, N and S. Heteroaryl groups within the scope of this definition include but are not limited to: acridinyl, carbazolyl, cinnolinyl, quinoxalinyl, pyrrazolyl, indolyl, benzotriazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl, tetrahydroquinoline. As with the definition of heterocycle below, “heteroaryl” is also understood to include the N-oxide derivative of any nitrogen-containing heteroaryl. In cases where the heteroaryl substituent is bicyclic and one ring is non-aromatic or contains no heteroatoms, it is understood that attachment is via the aromatic ring or via the heteroatom containing ring, respectively.

The term “heterocycle” or “heteroaryl” or “heterocycloalkyl” refers to a five-member to ten-member, fully saturated or partially unsaturated nonaromatic heterocyclic groups containing at least one heteroatom such as O, S or N. The most frequent examples are piperidinyl, morpholinyl, piperazinyl, pyrrolidinyl or pirazinyl. Attachment of a heterocycle substituent can occur via a carbon atom or via a heteroatom.

Moreover, the alkyl, alkoxy, aryl, aryloxy and heteroaryl groups described above can be “unsubstituted” or “substituted.” The term “substituted” is intended to describe moieties having substituents replacing a hydrogen on one or more atoms, e.g. C, O or N, of a molecule. Such substituents can independently include, for example, one or more of the following: straight or branched alkyl (preferably C₁-C₅), cycloalkyl (preferably C₃-C₈), alkoxy (preferably C₁-C₆), thioalkyl (preferably C₁-C₆), alkenyl (preferably C₂-C₆), alkynyl (preferably C₂-C₆), heterocyclic, carbocyclic, aryl (e.g., phenyl), aryloxy (e.g., phenoxy), aralkyl (e.g., benzyl), aryloxyalkyl (e.g., phenyloxyalkyl), arylacetamidoyl, alkylaryl, heteroaralkyl, alkylcarbonyl and arylcarbonyl or other such acyl group, heteroarylcarbonyl, or heteroaryl group, (CR′R″)₀₋₃NR′R″ (e.g., —NH₂), to (CR′R″)₀₋₃CN (e.g., —CN), —NO₂, halogen (e.g., —F, —Cl, —Br, or —I), (CR′R″)₀₋₃C(halogen)₃ (e.g., —CF₃), (CR′R″)₀₋₃CH(halogen)₂, (CR′R″)₀₋₃CH₂(halogen), (CR′R″)₀₋₃CONR′R″, (CR′R″)₀₋₃(CNH)NR′R″, (CR′R″)₀₋₃S(O)₁₋₂NR′R″, (CR′R″)₀₋₃CHO, (CR′R″)₀₋₃O(CR′R″)₀₋₃H, (CR′R″)₀₋₃S(O)₀₋₃R′ (e.g., —SO₃H, —OSO₃H), (CR′R″)₀₋₃O(CR′R″)₀₋₃H (e.g., —CH₂OCH₃ and —OCH₃), (CR′R″)₀₋₃S(CR′R″)₀₋₃H (e.g., —SH and —SCH₃), (CR′R″)₀₋₃OH (e.g., —OH), (CR′R″)₀₋₃COR′, (CR′R″)₀₋₃(substituted or unsubstituted phenyl), (CR′R″)₀₋₃(C₃-C₈ cycloalkyl), (CR′R″)₀₋₃CO₂R′ (e.g., —CO₂H), or (CR′R″)₀₋₃OR′ group, or the side chain of any naturally occurring amino acid; wherein R′ and R″ are each independently hydrogen, a C₁-C₅ alkyl, C₂-C₅ alkenyl, C₂-C₅ alkynyl, or aryl group.

The terms “alkyl-aryl”, “alkyl-heteroaryl” and “alkyl-heterocycloalkyl” refer to an alkyl substituent that is substituted by an alkyl, heteroaryl or heterocycloalkyl group, respectively. For example, the term alkyl-aryl includes, but is not limited to the following substituent:

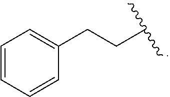 [SMILES: *CCc1ccccc1]

The term “amine” or “amino” should be understood as being broadly applied to both a molecule, or a moiety or functional group, as generally understood in the art, and may be primary, secondary, or tertiary. The term “amine” or “amino” includes compounds where a nitrogen atom is covalently bonded to at least one carbon, hydrogen or heteroatom. The terms include, for example, but are not limited to, “alkyl amino,” “arylamino,” “diarylamino,” “alkylarylamino,” “alkylaminoaryl”, “arylaminoalkyl,” “alkaminoalkyl,” “amide,” “amido,” and “aminocarbonyl.” The term “alkyl amino” comprises groups and compounds wherein the nitrogen is bound to at least one additional alkyl group. The term “dialkyl amino” includes groups wherein the nitrogen atom is bound to at least two additional alkyl groups. The term “arylamino” and “diarylamino” include groups wherein the nitrogen is bound to at least one or two aryl groups, respectively. The term “alkylarylamino,” “alkylaminoaryl” or “arylaminoalkyl” refers to an amino group which is bound to at least one alkyl group and at least one aryl group. The term “alkaminoalkyl” refers to an alkyl, alkenyl, or alkynyl group bound to a nitrogen atom which is also bound to an alkyl group.

By the term “protecting group” as used herein, it is meant that a particular functional moiety, e.g., O, S, or N, is temporarily blocked so that a reaction can be carried out selectively at another reactive site in a multifunctional compound. In preferred embodiments, a protecting group reacts selectively in good yield to give a protected substrate that is stable to the projected reactions; the protecting group must be selectively removed in good yield by readily available, preferably nontoxic reagents that do not attack the other functional groups; the protecting group forms an easily separable derivative (more preferably without the generation of new stereogenic centers); and the protecting group has a minimum of additional functionality to avoid further sites of reaction. As detailed herein, oxygen, sulfur, nitrogen and carbon protecting groups may be utilized. For example, in certain embodiments, as detailed herein, certain exemplary oxygen protecting groups are utilized. These oxygen protecting groups include, but are not limited to methyl ethers, substituted methyl ethers (e.g., MOM (methoxymethyl ether), MTM (methylthiomethyl ether), BOM (benzyloxymethyl ether), PMBM or MPM, para methoxybenzyloxymethyl ether), to name a few), substituted ethyl ethers, substituted benzyl ethers, silyl ethers (e.g., TMS (trimethylsilyl ether), TES (triethylsilylether), TIPS (triisopropylsilyl ether), TBDMS (t-butyldimethylsilyl ether), tribenzyl silyl ether, TBDPS (t-butyldiphenyl silyl ether), to name a few), esters (e.g., formate, acetate, benzoate (Bz), trifluoroacetate, dichloroacetate, to name a few), carbonates, cyclic acetals and ketals.

Tautomers of these compounds can be depicted in at least two ways as illustrated below:

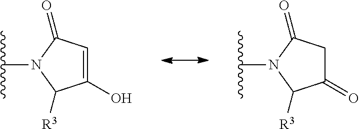

illustrative Structures, Showing Keto-Enol Tautomers

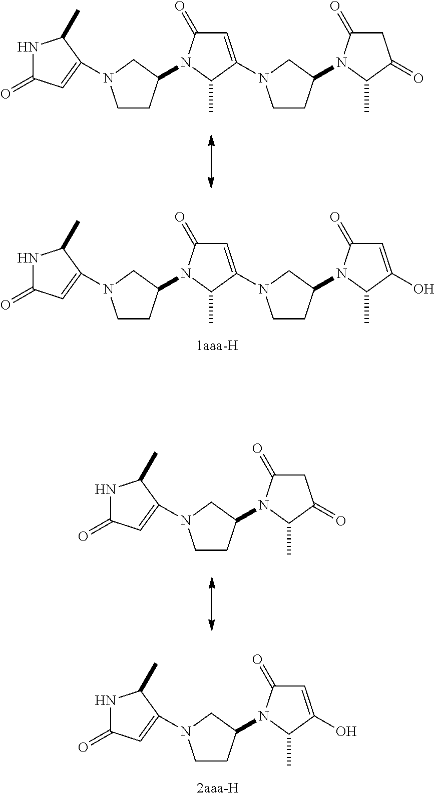

### Part I. Pyrrolinone-Pyrrolidine Oligomers

Provided herein are compounds useful as peptidomimetics. The compounds have preferred conformations that overlay well with amino acid residues in three different types of helix (3₁₀, α and π), in β-strands, and in both parallel- and antiparallel β-sheets. Compounds that overlay with amino acid residues found at the interface of protein-protein interactions have the ability to perturb and/or disrupt those interactions when the compounds are contacted with the protein(s).

### Synthesis of Compounds

For the preparation of scaffold 1 trans-4-hydroxyproline was decarboxylated yield (R)-3-hydroxypyrrolidine. That pyrrolidine was N-protected to give the starting material indicated in Scheme 1, below. Nucleophilic displacement on a triflate-derivative of this (under conditions optimized to avoid elimination) gave the amino esters 4. X-ray analysis of 4d.HCl indicated its formation occurred via a single inversion. The crystalline hydrochloride salts of 4 were reacted with Bestmann's ylide to give the pyrrolinone 5. Hydrogenolysis, then condensation of the free pyrrolidine-NH with 5-substituted 2,4-pyrrolidinediones (tetramic acids) 7 gave the featured trimers 1. The tetramic acid derivatives 7 are useful starting materials because they can be prepared from N-Boc-protected amino acids via a one-pot procedure that affords tens of gram amounts without chromatography. NMR and X-ray analysis of compound 6d indicated its formation was not complicated by epimerization. Condensation of 6 with C-deprotected derivatives of 2 gave the 1-^{t}Bu, Scheme 1.

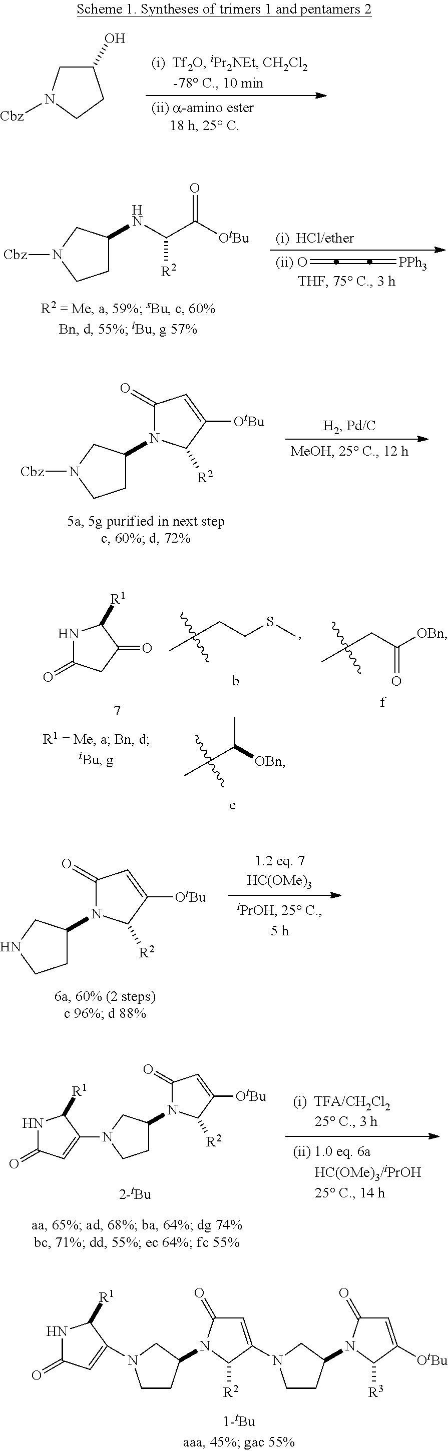

Compounds Overlaid with Secondary Structure Motifs

NMR studies to detect preferred conformations in these types of molecules are inappropriate due to conformational averaging. Consequently, two complementary molecular modeling methods were used. Quenched molecular dynamics (QMD) probes thermodynamic accessibilities of conformational states, as described previously. Briefly, this technique generates 600 minimized structures; ones that are energetically below a user-defined cut-off from the minimum energy conformer located (here 3.0 kcal/mol) are clustered into families based on RMS (root mean squared) deviations from user-defined atoms (0.5 Å). Matching Cα−Cβ bond vectors forms a good basis for measuring fit to secondary structures. Thus, preferred conformations of scaffolds may be defined by frameworks with only Me-side chains (e.g., Ala-analogs, like 2aa-H and 1aaa-H). For this reason, preferred conformers of 2aa-H and 1aaa-H were clustered based on Cα−Cβ coordinates, and representative members of each cluster were tested for fit on Cα−Cβ atom positions of ideal secondary structure motifs.

In this way, 2aa-H was calculated to have 582 conformers (18 families) and 1aaa-H was calculated to have 490 conformers (166 families) within 3.0 kcal/mol and 0.5 Å RMSD. The lowest energy structures from each family were each overlaid on the ideal secondary structures.

In the event, the best match for 1aaa-H was with three residues of a sheet-turn-sheet motif (1.93 kcal/mol above the minimum energy conformer; RMSD: 0.46 Å). Moreover, we found one example of a protein-protein interaction (between monomers in the RAD52 undecamer) where 1aaa-H matched three side-chains with an RMSD of only 0.14 Å.

The next milestone in this study was to check that the different conformers are kinetically accessible. To do this, a density functional theory (DFT) method was used to investigate interconversion between the preferred states of 6a. A maximum energy barrier of 5.10 kcal/mol was calculated using this method. This indicates conformers of 6a should rapidly interconvert on the 1H NMR time scale, and experimentally this was shown to be the case.

A synthesis of one compound corresponding to scaffold 3 was developed to demonstrate that both heterocyclic rings in the “main-chain” of these peptidomimetics could be functionalized with amino acids. That route (Scheme 2 is similar to Scheme 1 except that a thioamide was introduced (9 to 10) then reduced to the amine (12 to 3)).

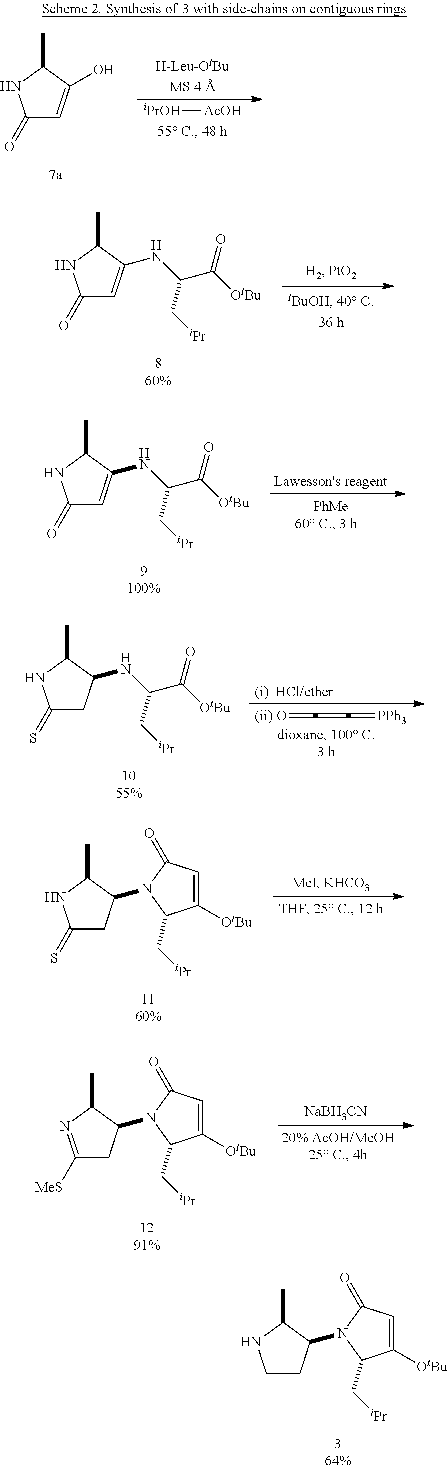

Extensive conformational analyses were performed for compound 3. Preferred conformers of 3 do not fit pairs of amino acid side-chains in secondary structures as well as conformers of 2aa-H does. The side chains in 3 on contiguous residues are constrained in ways that preclude good overlap on common secondary structure motifs. This is supported by the modeling studies shown here and X-ray crystallographic analyses of compound 11. Conversely, the 2aa-H has at least one extra significant degree of freedom, and this allows them to flex into conformations that match secondary structures well.

### In Vitro Assays—Inhibition of HIV-1 Protease Dimerization

In one aspect, provided herein is a method for inhibiting protein-protein interactions, comprising contacting the interacting proteins with a compound of formula (I). In one embodiment, the protein-protein interaction is a dimerization. In another embodiment, the protein is HIV-1 protease.

The following discussion of HIV-1 protease dimerization inhibition refers to the compounds as depicted and numbered in Scheme 3, below. These compounds have side chains corresponding to the HIV-1 dimerization interface, except that the cysteine side-chain (corresponding to Cys⁹⁵) was replaced by Ala. Previous studies have shown HIV-1 protease mutants wherein Cys⁹⁵ was replaced with Ala have almost the same K_{d} for the dimer dissociation, hence we used Ala instead of Cys⁹⁵ in syntheses.

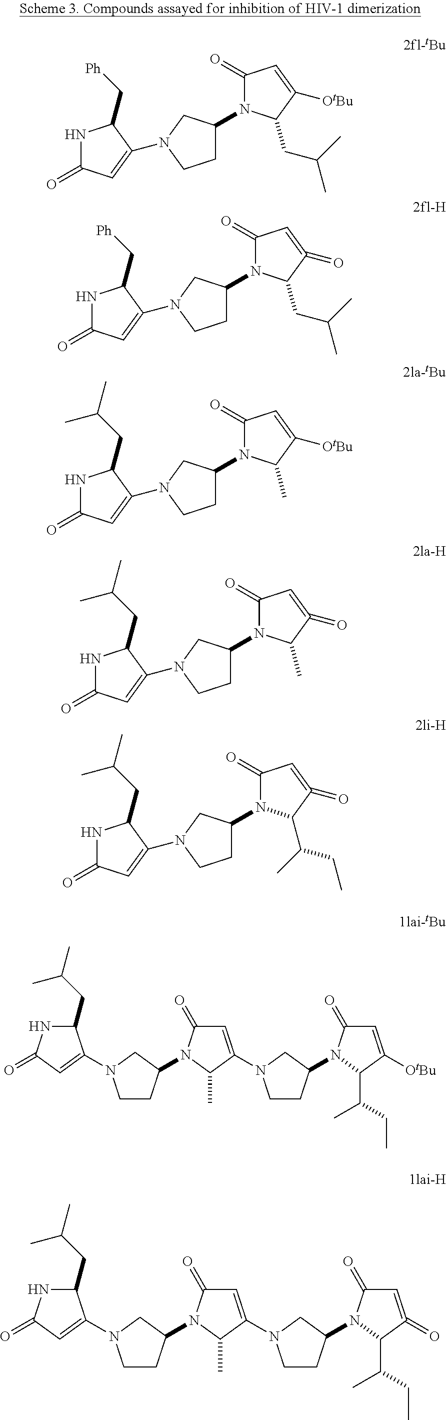

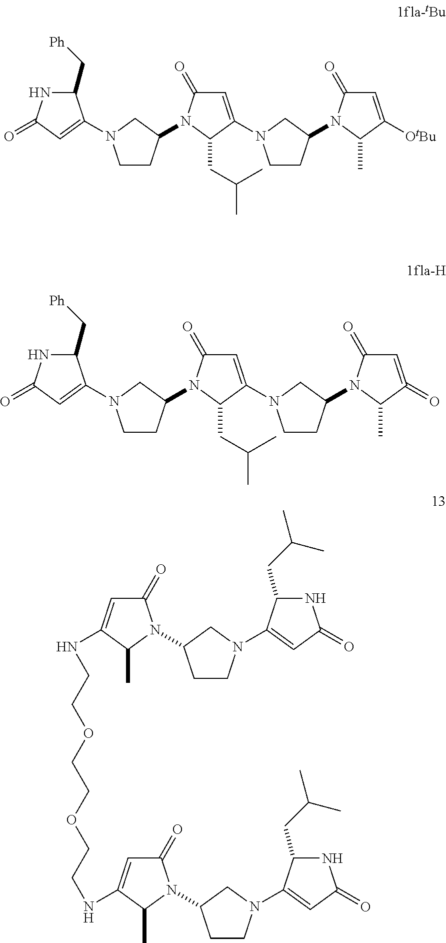

The HIV-1 protease inhibitory activities of the compounds were determined by a FRET method. HIV-1 protease stock solution was diluted with assay buffer (0.1 M sodium acetate, 1.0 M sodium chloride, 1.0 mM EDTA, 1.0 mM DTT, 10% DMSO, and 1.0 mg/mL BSA, pH 4.7). All inhibitors were dissolved in DMSO, and diluted to appropriate concentrations with deionized water such that the maximum conc. of DMSO in the buffer was 8.5%. EDANS/DABCYL-based FRET peptide substrate (Ex/Em=340/490 nm) solution in SensoLyte® 490 HIV Protease Assay Kit (Cat. #71127) and HiLyte Fluor™ 488/QXL™ 520-based FRET peptide substrate (Ex/Em=490/520 nm) solution in SensoLyte® 520 HIV Protease Assay Kit (Cat. #71147) were purchased from Anaspec. We needed to use these two different substrates because the compounds without C-protection (throughout this means protection on the oxygen at the C-terminus, a terminal OH) have weak fluorescence that interferes with that from the EDANS/DABCYL-based FRET peptide substrate (Ex/Em=340/490 nm) so an alternative substrate was used. Concentrations of the substrates are proprietary information of Anaspec, hence we followed a protocol for the substrate preparation in the assay kit.

For the determination of IC₅₀ values, HIV-1 protease (40 μL, 10.2 nM final concentration) and inhibitors (10 μL) were incubated for 15 min at 25° C. Substrate solutions (50 μL) in the buffer were added into the incubated solution to initiate the reaction. EDANS/DABCYL-based FRET peptide substrate was used for C-protected inhibitors, and HiLyte Fluor™ 488/QXL™ 520-based FRET peptide substrate was used for deprotected inhibitors. The total assay volume was 100 μL. Fluorescence was monitored for 5 min at 30° C. in a fluorescence microplate reader (BioTek) at Ex/Em=340/490 nm for ^{t}Bu-protected inhibitors, and Ex/Em=490/520 nm for deprotected inhibitors. The initial velocities were plotted against log [inhibitor] and a sigmoidal curve was fitted to the data points using Graphpad Prism 5 software to obtain IC₅₀ values.

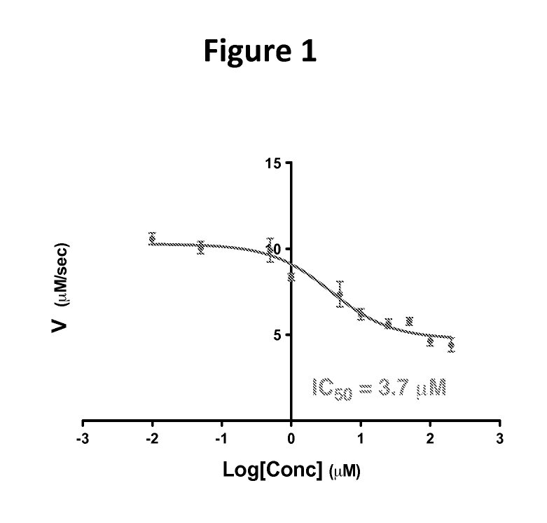

We obtained the best IC₅₀ (3.7±0.3 μM) for inhibition of HIV-1 protease from 1lai-H (see FIG. 1; other results are summarized in Table 2). Overall, the compounds with three side-chains gave better inhibition effects than those with only two, and C-deprotected compounds 1lai-H and 1fla-H showed better inhibition than protected forms (1lai-^{t}Bu and 1fla-^{t}Bu). Compound 2-la-H did not show any inhibition but the corresponding bivalent compound 13 showed two times better inhibition than trimer 2la-^{t}Bu.

To explore if these compounds inhibit dimerization of HIV-1 protease, we carried out a Zhang-Poorman kinetic assay. If a compound acts as a dimerization inhibitor, the Zhang-Poorman plot gives a line with a slope similar to that obtained for the uninhibited control but with a different intercept; active-site inhibitors yield different slopes compared with the uninhibited control. For this assay, HIV-1 protease was used at concentrations from 0.6 to 5.1 nM. Substrate solutions were diluted to 1/4 solution from the original solution, and then used for the kinetic assay. HIV-1 protease (40 μL) was incubated with or without an inhibitor (10 μL) at the desired concentration for 15 min at 25° C. The diluted substrate solution (50 μL) was added to the incubated solution. Fluorescence was monitored for 15 min at 30° C. in a fluorescence microplate reader (BioTek) at Ex/Em=340/490 nm for C-protected inhibitors, and Ex/Em=490/520 nm for deprotected inhibitors.

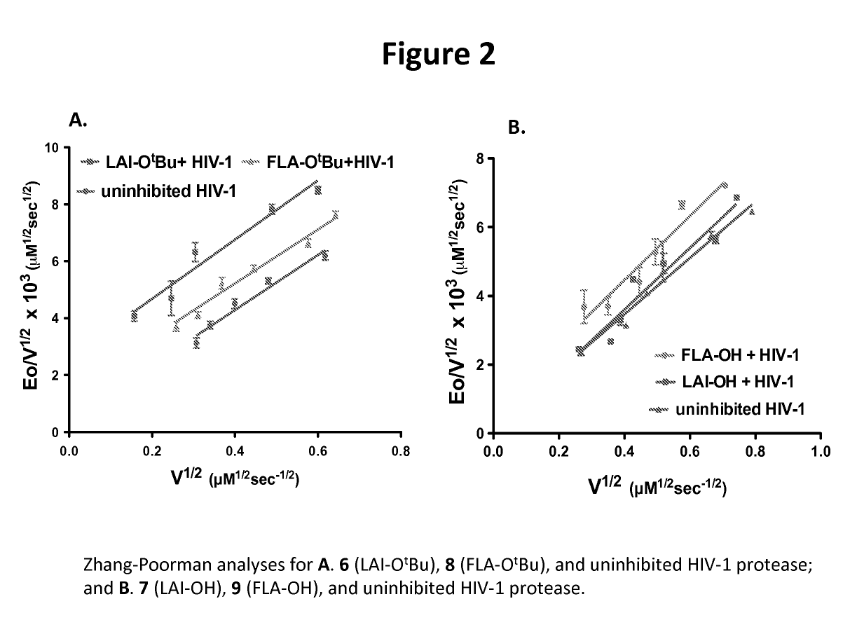

FIG. 2A shows Zhang-Poorman plots for C-protected compounds, 1lai-^{t}Bu and 1fla-^{t}Bu. Slopes for 1lai-^{t}Bu (10.4±1.0) and 1fla-^{t}Bu (9.5±0.6) are similar with one for uninhibited HIV-1 (9.7±0.7) with significantly different y-intercepts (1lai-^{t}Bu y-intercepts 2.6, 1fla-^{t}Bu y-intercepts 1.4, control y-intercepts 0.42); these observations indicate the compounds are acting as dimerization inhibitors (see below). The deprotected compounds 1lai-H and 1fla-H also showed similar patterns in FIG. 2B. Slopes for 1lai-H (8.2±0.6) and 1fla-H (9.3±1.2) compared with uninhibited HIV-1 (9.0±0.6) have different y-intercepts (1lai-H y-intercepts 0.19, 1fla-H y-intercepts 0.73, control y-intercepts 0.013). y-Intercepts of uninhibited HIV-1 have different values between experiments for C-protected and deprotected compounds because the substrates used in the assay are different (see above). The results are consistent with 1-H-1-^{t}Bu (side-chains as indicated above) acting as dimerization inhibitors. Kᵢ values calculated from the y-intercepts using the Zhang-Poorman equation are summarized in Table 1. We note that it has been reported that inhibition of HIV-1 protease activities by dimerization inhibitors is dependent on the time of pre-incubation with the enzyme and inversely dependent on enzyme concentration; consequently, these factors must be standardized if comparing our data with those from different labs.

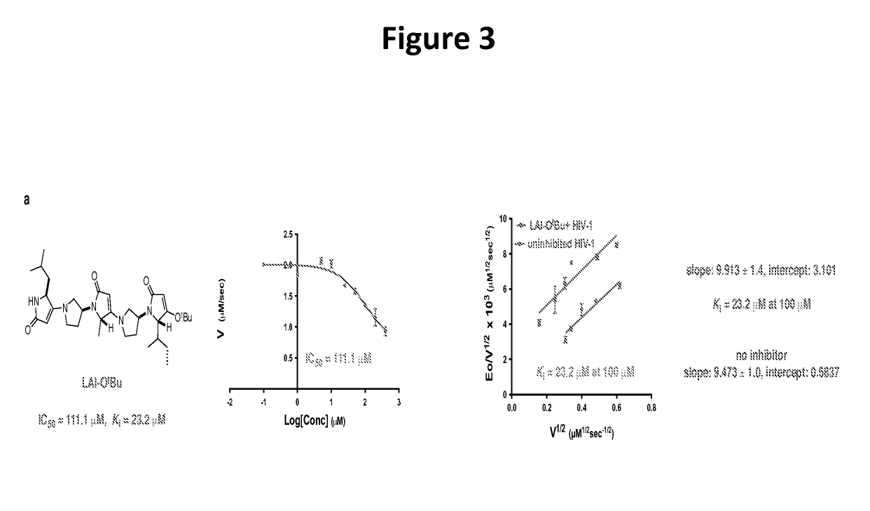

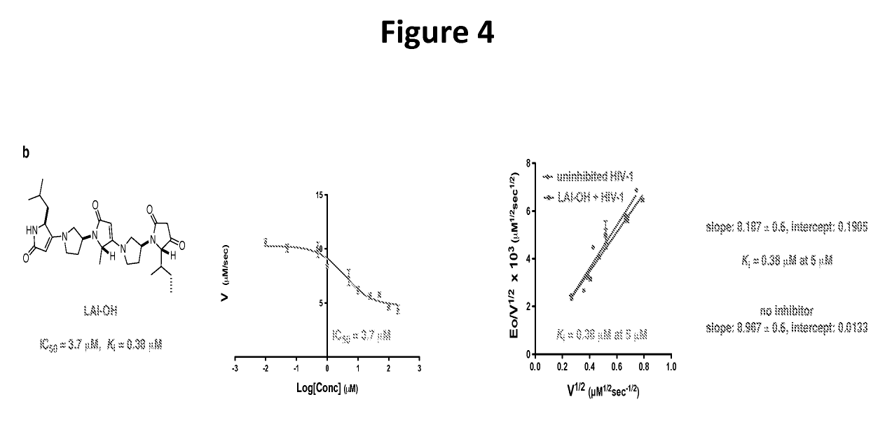

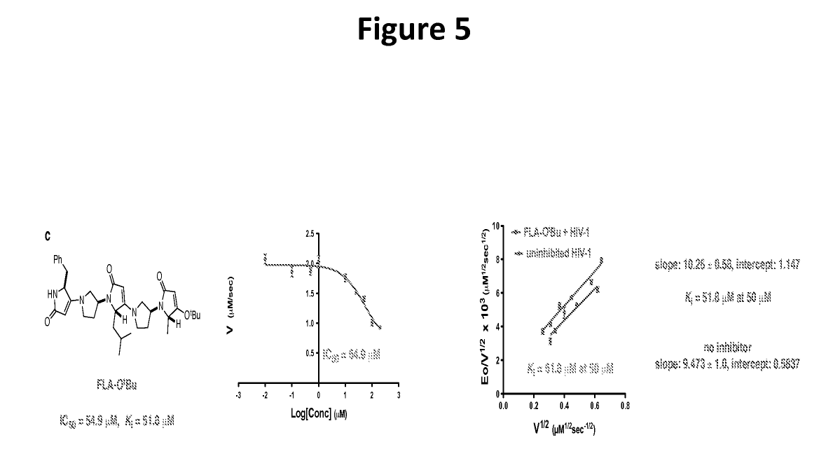

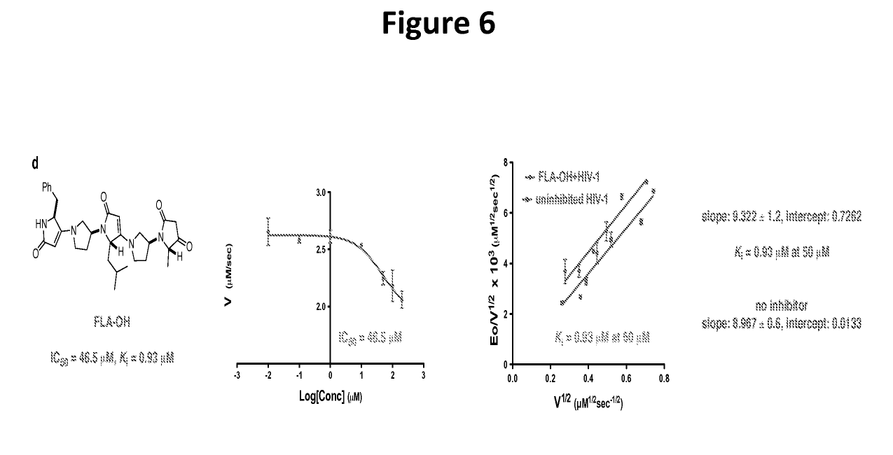

TABLE 1
Summary of IC₅₀ and Kᵢ Data For Compounds Indicated
(see FIGS. 3-6 for more illustrative data)

| entry | compound | IC₅₀ (μM) | Kᵢ (μM) |
|---|---|---|---|
| 1 | 2fl-^{t}Bu | 516.3 | |
| 2 | 2fl-H | 418.7 | |
| 3 | 2la-^{t}Bu | 176.4 ± 16 | |
| 4 | 2la-H | — | — |
| 5 | 2li-H | 623.2 | |
| 6 | 1lai-^{t}Bu | 111.1 ± 18 | 19.4 ± 4.1 |
| 7 | 1lai-H | 3.7 ± 0.4 | 0.38 ± 0.07 |
| 8 | 1fla-^{t}Bu | 54.9 ± 6 | 21.0 ± 2.1 |
| 9 | 1fla-H | 46.5 ± 8 | 0.93 ± 0.3 |
| 10 | 13 | 84.4 ± 10 | |

### Part II. Piperidine-Piperidinone Oligomers

A series of compounds 14 have been prepared for these studies.

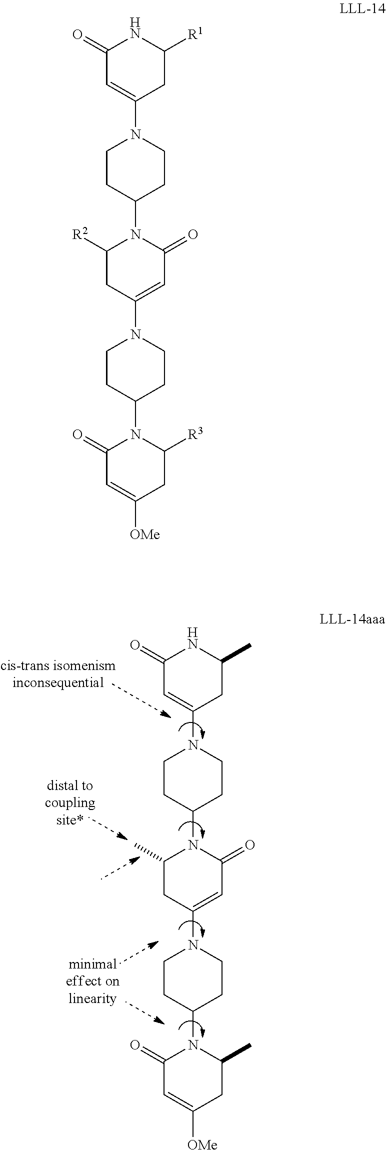

Briefly, preparation of compounds 14 requires first synthesis of “electrophilic cap” components 15 as shown in Scheme 4a.

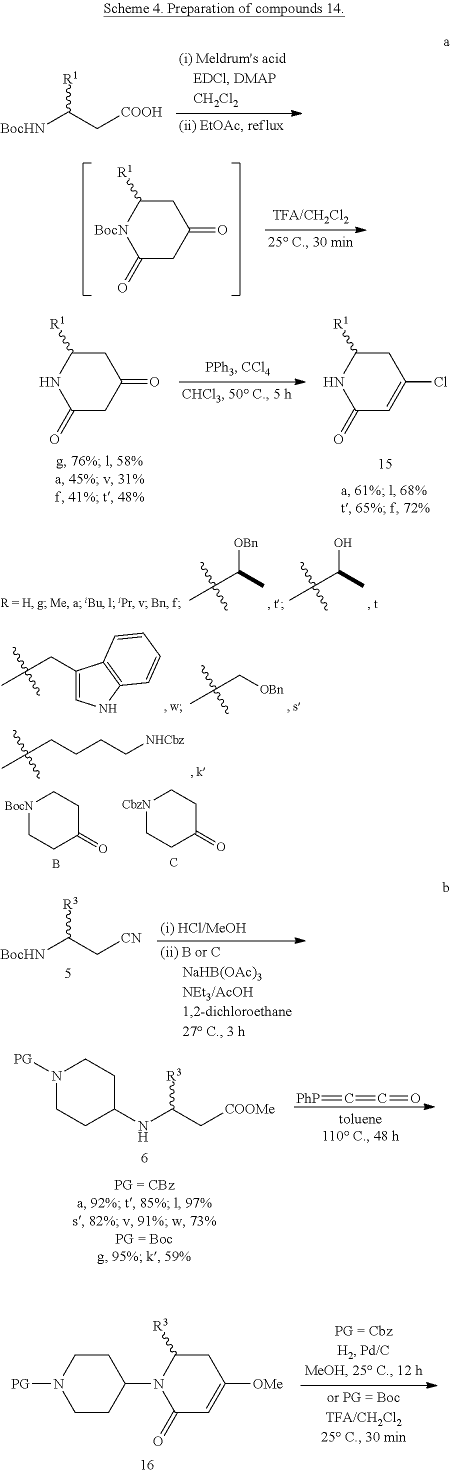

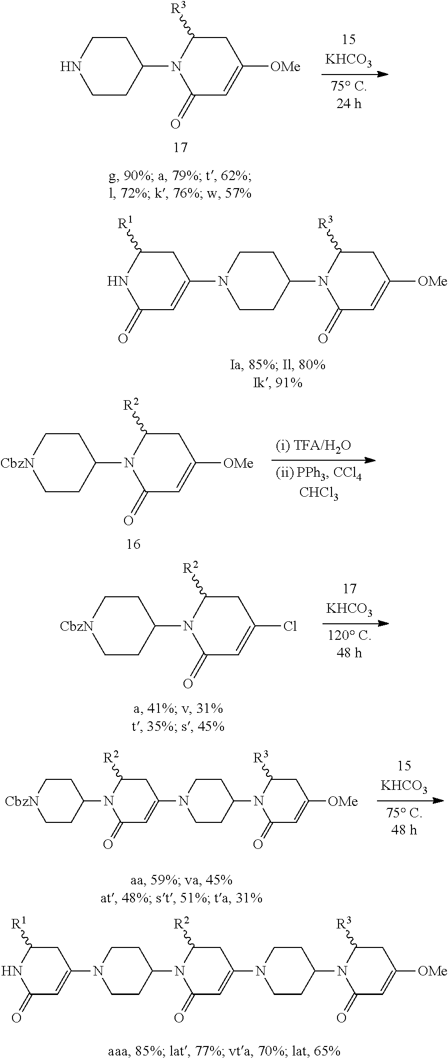

The nitriles starting materials at the beginning of Scheme 4b are intermediates in the syntheses of the β-amino acids used in syntheses of the b-amino acid starting materials in Scheme 4a. These were simultaneously N-deprotected and hydrolyzed, then reductively coupled with the known, and commercially available, synthons B or C (Scheme 4a) to give the amines shown. Reaction of these amines with Bestmann's glide gave the protected intermediates 16 and N-deprotection of these gave the nucleophiles 17. Amines 17 were then condensed with the electrophiles 15 to give scaffolds bearing two side-chains as shown.

Intermediates 16 were alternatively C-deprotected, then converted to vinylogous chlorides then coupled with the nucleophiles 17 then with the electrophiles 15 to give extended systems that were then N-deprotected.

Overall, the syntheses of compounds 1 are divergent-convergent hinging on synthons 16; these can be converted to nucleo- or electrophiles that can be joined to elongate the scaffold. Fragments derived from 16 are similar to C- and N-protected amino acids in peptide syntheses.

Rates of permeation of compounds through Caco-2 cells can be predicted via QikProp as an indicator of cell permeability and oral bioavailability; rates of >20 nm·sec⁻¹ are widely considered to be favorable. This type of data for scaffolds 14 are one order of magnitude greater than systems 1, and two more the corresponding tripeptides.

### Experimental Conditions

In the context of this study, “simulation” refers to generation a series of three dimensional molecular conformations of a molecule (like 1aaa) in solution. These conformations are virtual three-dimensional representations of the shapes the molecule can adopt as the bonds within it that are free to rotate do so. These states are usually expressed as sets of Cartesian coordinates. A set of virtual conformations like this is called a “conformational ensemble”. Relative energies for each member of the conformational ensemble may be calculated. In actuality, the lowest energy conformational states will be significantly populated by real molecules existing in those shapes. Conformational states above, for instance, 3 kcal·mol⁻¹ from the lowest energy one will not be significantly populated by a percentage of the molecules. “Molecular simulation” here is the term given to any computer-based method to predict the lowest energy, most populated, members of a conformational ensemble for the featured substance.

Quenched molecular dynamics (QMD) was used for the molecular simulations performed in this work. Explicit atom representations were used throughout the study. The protein structure files (PSF) for all the peptidomimetics were built using Discovery Studio 2.5 (Accelrys Inc) using the CHARMm force field.

Quenched molecular dynamics (QMD) simulations were performed using the CHARMm force field. All molecules were modeled as neutral compounds in a dielectric continuum of 80 (simulating H₂O). Thus, the starting conformers were minimized using 3000 steps of conjugate gradient. The minimized structures were then subjected to heating, equilibration, and dynamics simulation. Throughout, the equations of motions were integrated using the Verlet algorithm with a time step 1 fs. Each peptidomimetic was heated to 1000 K over 10 ps and equilibrated for another 10 ps at 1000 K, then molecular dynamics runs were performed for a total time of 600 ps with trajectories saved every 1 ps. The resulting 600 structures were thoroughly minimized using 1000 steps of SD followed by 3000 steps of conjugate gradient. Structures with energies less than 3.0 kcal mol⁻¹ relative to the global minimum were selected for further analysis.

The VMD package was used to display, overlay, and classify the selected structures into conformational groups. The best clustering was obtained using a grouping method based on calculation of RMS deviation of a subset of atoms, in this study these were the Cα−Cβ atoms. Thus, threshold cutoff values 0.3 Å were selected to obtain families with reasonable homogeneity. The lowest energy conformation from each family was considered to be a typical representative of the family as a whole.

Throughout this document the term “overlay” is used to describe the act of superimposing a set of three dimensional coordinates (eg six Cartesian coordinates representing the positions of three sets of Cα−Cβ atoms) with an identical number of three dimensional coordinates (eg another set of six Cartesian coordinates representing the positions of three sets of Cα−Cβ atoms) so that they correspond to each other as closely as possible.

Throughout this document the term “goodness-of-fit” is used to describe a quantitative measure of how well two sets of coordinates can be overlayed. Typically, goodness of fit is expressed as root mean squared deviation (RMSD, measured in A), though other parameters can be used or conceived to give an alternative measure of goodness of fit.

Reaction path calculations were performed at the B3LYP level of theory with the 6-31G(d′) basis set, and a polarized continuum solvation model with a dielectric of H₂O (∈=78.3553). All B3LYP calculations were performed using Gaussian 03.5. The energy barriers for the compounds 6a′ and 6d were calculated as rotations of the bonds (red arrows). ΔG^{o} values were shown in kcal/mol. For 6a′, DFT calculations showed one more low energy conformer, B′ as a minor conformer.

### Synthesis of Compounds

### General Experimental Methods:

All reactions were carried out under an inert atmosphere (nitrogen or argon where stated) with dry solvents under anhydrous conditions. Glassware for anhydrous reactions were dried in an oven at 140° C. for minimum 6 h prior to use. Dry solvents were obtained by passing the previously degassed solvents through activated alumina columns. Yields refer to chromatographically and spectroscopically (1H-NMR) homogeneous materials, unless otherwise stated. Reagents were purchased at a high commercial quality (typically 97% or higher) and used without further purification, unless otherwise stated. Analytical thin layer chromatography (TLC) was carried out on Merck silica gel plates with QF-254 indicator and visualized by UV, ceric ammonium molybdate, and/or potassium permanganate stains. Flash column chromatography was performed using silica gel 60 (Silicycle, 230-400 mesh) as per the Still protocol.1 1H and 13C spectra were recorded on a Varian Mercury or Inova spectrometer (300 MHz 1H; 75 MHz 13C) and were calibrated using residual non-deuterated solvent as an internal reference (CDCl3: 1H-NMR=7.26, 13C-NMR=77.16, DMSO-d6: 13C-NMR=39.52, CD3OD: 1H-NMR=3.31, 13C-NMR=49.00). The following abbreviations or combinations thereof were used to explain the multiplicities: s=singlet, d=doublet, t=triplet, q=quartet, m=multiplet, p=pentet, br=broad, app=apparent. IR spectra were recorded on an IRAffinity-1 Shimadzu spectrophotometer using NaCl plates. Melting points were recorded on an automated melting point apparatus (EZ-Melt, Stanford Research Systems) and are uncorrected. Optical rotations were obtained on a Jasco DIP-360 digital polarimeter at the D-line of sodium.

### General Procedures for Synthesis:

Procedure for generating α-amino esters from corresponding hydrochloride salts in high yields under mild conditions. The HCl salt (20 mmol) was suspended in 100 mL of 3:1 chloroform/isopropanol and transferred to a 500 mL reparatory funnel. Sodium carbonate solution (5%, 250 mL) was added and the organic layer is separated after extraction. The aqueous layer was extracted with three 50 mL portions of 3:1 chloroform/isopropanol. The combined organic layers was washed with brine (100 mL), dried over MgSO4, filtered and concentrated to afford the α-amino ester as a colorless liquid (>95%).

### General Procedure for X-Ray Structure Determination:

A Leica MZ 75 microscope was used to identify a suitable colorless multi-faceted crystal with very well defined faces with dimensions (max, intermediate, and min) 0.05 mm×0.03 mm×0.01 mm from a representative sample of crystals of the same habit. The crystal mounted on a nylon loop was then placed in a cold nitrogen stream maintained at 110 K.

A BRUKER D8-GADDS X-ray (three-circle) diffractometer was employed for crystal screening, unit cell determination, and data collection. The goniometer was controlled using the FRAMBO software suite. The sample was optically centered with the aid of a video camera such that no translations were observed as the crystal was rotated through all positions. The detector was set at 6.0 cm from the crystal sample (MWPC Hi-Star Detector, 512×512 pixel). The X-ray radiation employed was generated from a Cu sealed X-ray tube (K_{α}=1.54184 Å with a potential of 40 kV and a current of 40 mA) fitted with a graphite monochromator in the parallel mode (175 mm collimator with 0.5 mm mono-capillary optics). The rotation exposure indicated acceptable crystal quality and the unit cell determination was undertaken. 2100 data frames were taken at widths of 0.5° with an exposure time of 10 seconds. Over 6000 reflections were centered and their positions were determined. These reflections were used in the auto-indexing procedure to determine the unit cell. A suitable cell was found and refined by nonlinear least squares and Bravais lattice procedures and reported here in Table 1 No super-cell or erroneous reflections were observed. After careful examination of the unit cell, a standard data collection procedure was initiated. This procedure consists of collection of one hemisphere of data collected using omega scans, involving the collection 0.5° frames at fixed angles for φ, 2θ, and χ (2θ=−28°, χ=54.73°, 2θ=−90°, χ=54.73°), while varying omega. Addition data frames were collected to complete the data set. Each frame was exposed for 10 sec. The total data collection was performed for duration of approximately 24 hours at 110K. No significant intensity fluctuations of equivalent reflections were observed.

### Data Reduction, Structure Solution, and Refinement:

Integrated intensity information for each reflection was obtained by reduction of the data frames with the program SAINT. The integration method employed a three dimensional profiling algorithm and all data were corrected for Lorentz and polarization factors, as well as for crystal decay effects. Finally the data was merged and scaled to produce a suitable data set. The absorption correction program SADABS was employed to correct the data for absorption effects. Systematic reflection conditions and statistical tests for the data suggested the space group P2₁. A solution was obtained readily using SHELXTL (SHELXS). All non-hydrogen atoms were refined with anisotropic thermal parameters. The Hydrogen atoms bound to carbon were placed in idealized positions [C—H=0.96 Å, Uᵢₛₒ(H)=1.2×Uᵢₛₒ(C)]. The structure was refined (weighted least squares refinement on F²) to convergence. X-seed was employed for the final data presentation and structure plots.

### (R)-benzyl 3-hydroxypyrrolidine-1-carboxylate

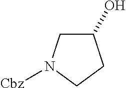 [SMILES: O=C(OCc1ccccc1)N1CC[C@@H](O)C1]

Procedure: To a stirred solution of (R)-pyrrolidin-3-ol maleate salt² (18.0 g, 88.3 mmol) in water (75 mL) was added sodium carbonate (47 g, 442 mmol, 5.0 equiv) portion-wise at 0° C. Benzyl chloroformate (15 mL, 106 mmol, 1.2 equiv) was added dropwise over 30 minutes using a syringe pump. The reaction was stirred at 25° C. for 4 h. Dichloromethane (250 mL) was added and the aqueous layer was separated. The organic layer was extracted once with water (50 mL) and brine (75 mL), dried over MgSO₄, filtered and concentrated. The residue was purified by column chromatography (SiO₂, 1:1 EtOAc/CH₂Cl₂) to afford the Cbz-protected pyrrolidin-3-ol in 78% yield. [α]²⁰−19.7 (c 1.0, MeOH); ¹H-NMR (300 MHz, CDCl₃) δ 7.39-2.29 (m, 5H), 5.18 (app s, 2H), 4.46 (br s, 1H), 3.61-3.42 (m, 4H), 2.45 (d, J=19.2 Hz, 1H), 2.02-1.95 (m, 2H)

### Compound 4f: (S)-benzyl 3-(((S)-1-(tert-butoxy)-1-oxo-3-phenylpropan-2-yl)amino)pyrrolidine-1-carboxylate

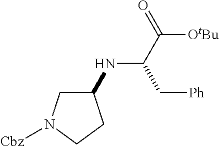 [SMILES: CC(C)(C)OC(=O)[C@H](Cc1ccccc1)N[C@H]1CCN(C(=O)OCc2ccccc2)C1]

Procedure: To a solution of Cbz-protected pyrrolidin-3-ol (2.58 g, 11.7 mmol) in dry dichloromethane (15 mL) at −78° C. was added diisopropylethyl amine (2.2 mL, 12.9 mmol, 1.1 equiv), dropwise. Freshly distilled (P₂O₅) triflic anyhydride (2.1 mL, 12.2 mmol, 1.05 equiv) was added using a syringe pump at rate of 4 mL/hr ensuring that the bath temperature does not exceed −70° C. The reaction mixture turned pink. On complete addition of triflic anyhydride, the reaction was stirred for 10 min. A solution of phenylalanine tert-butyl ester (3.89 g, 17.6 mmol, 1.5 equiv) in dichloromethane (15 mL) was then added at a rate of 30 mL/hr. The reaction was stirred for 10 minutes at −78° C., and allowed to warm to 25° C. During this time the reaction assumed an orange hue. After 18 h, the reaction mixture was transferred to a separatory funnel and diluted with dichloromethane (125 mL). The organic layer was extracted with saturated sodium bicarbonate (2×150 mL) and brine (1×100 mL). The organic layer was dried over MgSO₄, filtered and concentrated. The residue was purified by column chromatography (SiO₂, 1:5 ethyl acetate/dichloromethane; cerric ammonium molybdate stain and UV for visualization) to afford the product in 55% yield.

Note: NMR spectra show two conformers due to restricted rotation about the N—C═O bond of the Cbz group. ¹H-NMR (300 MHz, CDCl₃) δ 7.42-7.18 (m, 10H), 5.16-5.12 (m, 2H), 3.62-3.48 (m, 2H), 3.46-3.34 (m, 2H), 3.32-3.24 (m, 1H), 3.22-3.04 (m, 2H), 2.98-2.80 (m, 2H), 2.08-1.88 (m, 1H), 1.78-1.60 (m, 1H), 1.40-1.34 (m, 9H).

### Compound 4f.HCl: (S)-benzyl 3-(((S)-1-(tert-butoxy)-1-oxo-3-phenylpropan-2-yl)amino)pyrrolidine-1-carboxylate hydrochloride

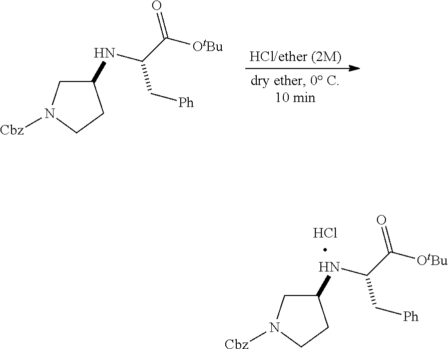 [SMILES: CC(C)(C)OC(=O)[C@H](Cc1ccccc1)N[C@H]1CCN(C(=O)OCc2ccccc2)C1 ; CC(C)(C)OC(=O)[C@H](Cc1ccccc1)N[C@H]1CCN(C(=O)OCc2ccccc2)C1]

Procedure: The amine was dissolved in dry ether (0.05 M) and cooled to 0° C. A solution of HCl(g)/ether (2 M, 1.1 equiv) was added drop wise. Upon complete precipitation, the solution was stirred for 5 min, and filtered. The precipitate was washed with dry ether to afford the pure product in >90% yield, which was recrystallized from ethanol.

Note: NMR spectra show two conformers due to restricted rotation about the N—C═O bond of the Cbz group. [α]²⁰+26.1 (c 0.5, MeOH); ¹H-NMR (300 MHz, CD₃OD) δ 7.41-7.20 (m, 10H), 5.14 (s, 2H), 4.28 (dd, J=9.9, 5.1 Hz, 1H), 4.04-3.94 (m, 1H), 3.92-3.78 (m, 1H), 3.72-3.58 (m, 2H), 3.51 (d, J=5.4 Hz, 1H), 3.46 (d, J=5.1 Hz, 1H), 3.04 (dd, J=13.8, 9.9 Hz, 1H), 2.48-2.32 (m, 1H), 2.28-208 (m, 1H), 1.28 (s, 9H).

### Compound 4i: S)-benzyl 3-(((2S,3S)-1-(tert-butoxy)-3-methyl-1-oxopentan-2-yl)amino)pyrrolidine-1-carboxylate

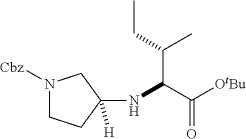

Procedure: As described before for (S)-benzyl 3-(((S)-1-(tert-butoxy)-1-oxo-3-phenylpropan-2-yl)amino)pyrrolidine-1-carboxylate. Column chromatography was performed using 10% ethyl acetate in dichloromethane to afford the product in 60% yield.

Note: NMR spectra show two conformers due to restricted rotation about the N—C═O bond of the Cbz group. ¹H-NMR (300 MHz, CDCl₃) δ 7.42-7.30 (m, 5H), 5.12 (s, 2H), 3.66-3.48 (m, 2H), 3.48-3.39 (m, 1H), 3.27-3.14 (m, 2H), 2.97-2.85 (m, 1H), 2.08-1.98 (m, 1H), 1.80-1.66 (m, 2H), 1.64-1.52 (m, 1H), 1.47 (m, 9H), 1.22-1.10 (m, 1H), 0.93-0.89 (m, 6H). MS (ESI) m/z calcd for (M+H)⁺ C₂₂H₃₅N₂O₄ 391.25. found 391.26.

### Compound 4i.HCl: Characterization of the hydrochloride salt

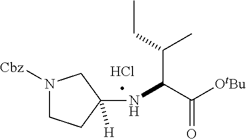

Procedure: As described before for compound 4f.HCl. The product was re-crystallized from hot MeCN (˜28 mL/gram).

Note: NMR spectra show two conformers due to restricted rotation about the N—C═O bond of the Cbz group. [α]²°+24.4 (c 0.5, MeOH); ¹H-NMR (300 MHz, CD₃OD) δ 7.37-7.28 (m, 5H), 5.14 (s, 2H), 4.00 (m, 1H), 3.98-3.78 (m, 2H), 3.72-3.60 (m, 1H), 3.60-3.40 (m, 2H), 2.45-2.28 (m, 1H), 2.22-2.06 (m, 1H), 1.72-1.60 (m, 1H), 1.54 (s, 9H), 1.50-1.36 (m, 2H), 1.08-0.98 (m, 6H).

### Compound 4a: (S)-benzyl 3-(((S)-1-(tert-butoxy)-1-oxopropan-2-yl)amino)pyrrolidine-1-carboxylate

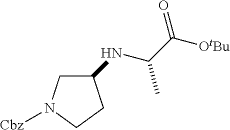 [SMILES: C[C@H](N[C@H]1CCN(C(=O)OCc2ccccc2)C1)C(=O)OC(C)(C)C]

Procedure: As described before for (S)-benzyl 3-(((S)-1-(tert-butoxy)-1-oxo-3-phenylpropan-2-yl)amino)pyrrolidine-1-carboxylate. The residue was purified by column chromatography (SiO₂, 1:2 ethyl acetate/dichloromethane; Cerric ammonium molybdate stain and UV for visualization) to afford the product in 59% yield.

Note: NMR spectra show two conformers due to restricted rotation about the N—C═O bond of the Cbz group. ¹H-NMR (300 MHz, CDCl₃) δ 7.40-7.26 (m, 5H), 5.11 (m, 2H), 4.40 (br s, 1H), 3.66-3.45 (m, 3H), 3.44-3.33 (m, 1H), 3.31-3.05 (m, 2H), 2.02-1.84 (m, 1H), 1.78-1.58 (m, 1H), 1.48 (s, 9H), 1.22 (d, J=6.0 Hz, 3H). MS (ESI) m/z calcd for C₁₉H₂₉N₂O₄ (M+H)⁺ 349.20. found 349.20.

### Compound 4a.HCl

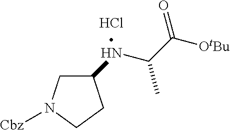

Procedure: As described before for compound 4f.HCl. The product was re-crystallized from hot MeCN (˜10 mL/gram).

Note: NMR spectra show two conformers due to restricted rotation about the N—C═O bond of the Cbz group. [α]²⁰−1.8 (c 0.5, MeOH); ¹H-NMR (300 MHz, CD₃OD) δ 7.44-7.31 (m, 5H), 5.19 (s, 2H), 4.14 (q, J=7.2 Hz, 1H), 4.08-3.94 (m, 1H), 3.94-3.78 (m, 1H), 3.75-3.43 (m, 3H), 2.51-2.34 (m, 1H), 2.27-2.08 (m, 1H), 1.60 (d, J=7.5 Hz, 3H), 1.56 (s, 9H)

### Compound 4l.HCl: (S)-benzyl 3-(((S)-1-(tert-butoxy)-4-methyl-1-oxopentan-2-yl)amino)pyrrolidine-1-carboxylate

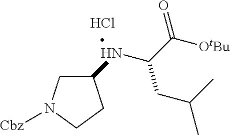

Procedure: As described before for compound 4f.HCl. The product was re-crystallized from hot MeCN (70 mL/gram); ¹H-NMR (300 MHz, CD₃OD) δ 7.43-7.27 (m, 5H), 5.14 (app s, 2H), 4.02-3.74 (m, 3H), 3.68-3.40 (m, 3H), 2.44-2.27 (m, 1H), 2.23-2.03 (m, 1H), 1.88-1.70 (m, 3H), 1.54 (s, 9H), 1.07-0.98 (m, 6H).

General Procedure for Hydrogenation of Substrates 4.HCl to Afford Diamine Derivatives 4′

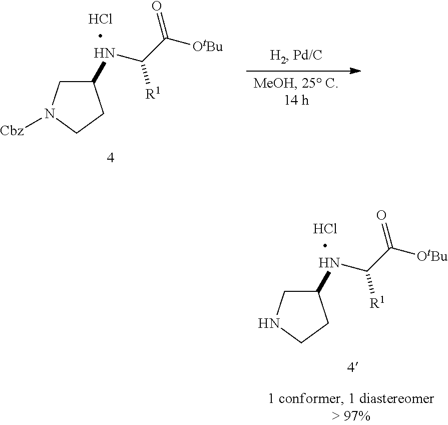

To a stirred solution of the starting material (4.HCl, 0.07 mmol) in MeOH (1 mL) was added 10% palladium on carbon (15 mg, 0.2 eq Pd) under a stream of N₂. The reaction was evacuated and re-filled with N2 and placed under an atmosphere of H₂ (balloon) for 14 h. The reaction was filtered using a small pipet plug of SiO₂. The plug was washed with MeOH (4 mL) and the combined eluent was concentrated to afford the pure products (4′) in >97% yield.

### Compound 4′f: (S)-tert-butyl 3-phenyl-2-((S)-pyrrolidin-3-ylamino)propanoate hydrochloride

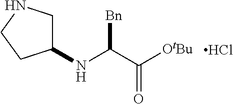 [SMILES: CC(C)(C)OC(=O)[C@H](Cc1ccccc1)N[C@H]1CCNC1 ; Cl]

¹H-NMR (300 MHz, CDCl₃) δ 7.31-7.13 (m, 5H), 3.37-3.27 (m, 4H), 3.24-3.10 (m, 2H), 2.99 (dd, J=13.4, 5.9 Hz, 1H), 2.84 (dd, J=13.2, 7.8 Hz, 1H), 2.16-2.02 (m, 1H), 1.88-1.73 (m, 1H), 1.32 (s, 9H); HRMS (ESI) m/z calcd for C₁₇H₂₇N₂O₂ (M+H)⁺ 291.2072. found 291.2079 (2.2 ppm).

### Compound 4′a: (S)-tert-butyl 2-((S)-pyrrolidin-3-ylamino)propanoate hydrochloride

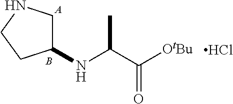

Procedure: As per the general procedure for hydrogenation of 4.HCl. ¹H-NMR (300 MHz, CDCl₃) δ 3.58-3.42 (m, 2H), 3.42-3.30 (m, 1H), 3.27-3.11 (m, 3H), 2.21-2.02 (m, 1H), 1.90-1.75 (m, 1H), 1.44 (s, 9H), 1.24 (d, J=6.9 Hz, 3H).

### Compound 4′i: (2S,3S)-tert-butyl 3-methyl-2-((S)-pyrrolidin-3-ylamino)pentanoate hydrochloride

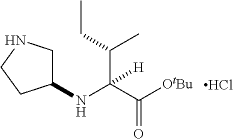 [SMILES: CC[C@H](C)[C@H](N[C@H]1CCNC1)C(=O)OC(C)(C)C ; Cl]

Procedure: As per the general procedure for hydrogenation of 4.HCl

¹H-NMR (300 MHz, CDCl₃) δ 3.52-3.19 (m, 4H), 3.07 (dd, J=11.6, 3.8 Hz, 1H), 2.85 (d, J=5.4 Hz, 1H), 2.18-2.02 (m, 1H), 1.85-1.72 (m, 1H), 1.65-1.43 (m, 1H), 1.42-1.36 (m, 10H), 1.19-1.02 (m, 1H), 0.90-1.82 (m, 6H).

### Compound 5f: (S)-benzyl 3-((S)-2-benzyl-3-(tert-butoxy)-5-oxo-2,5-dihydro-1H-pyrrol-1-yl)pyrrolidine-1-carboxylate

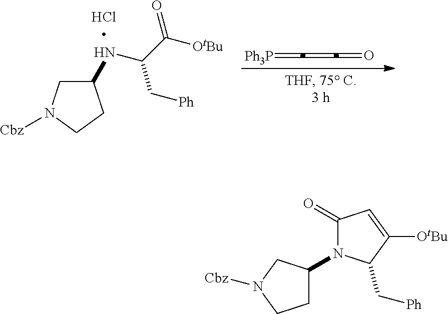

Procedure: The re-crystallized hydrochloride salt (2.8 g, 6.1 mmol) was suspended in dry THF (50 mL) and heated to 75° C. under an Argon atmosphere. Bestmann's ylide (2× re-crystallized from PhMe, 2.21 g, 7.32 mmol, 1.2 equiv) was added in one portion. After 30 min, a second portion of Bestmann's ylide (368 mg, 1.22 mmol, 0.2 equiv) was added, and this process was repeated four additional times at 15 min intervals to complete the addition of 2.2 equiv of ylide. The reaction was monitored by NMR spectroscopy. After completion of reaction (˜3 h), the solvent was evaporated. Ether (150 mL) was added to the residue and stirred for 3 h. The ether layer was decanted, and concentrated to obtain the crude product contaminated with Ph₃PO. The product was isolated by flash chromatography (5-10% acetone/dichlormethane) in 72% yield.

Note: NMR spectra show two conformers due to restricted rotation about the N—C═O bond of the Cbz group. ¹H-NMR (300 MHz, CDCl₃) δ 7.40-7.12 (m, 10H), 5.18-5.12 (m, 2H), 4.90 (app s), 4.18-4.02 (m, 2H), 3.84-3.74 (m, 1H), 3.74-3.58 (m, 2H), 3.42-3.28 (m, 1H), 3.16-2.96 (m, 2H), 2.62-2.38 (m, 1H), 2.18-2.02 (m, 1H), 1.38-1.34 (s, 9H); HRMS (ESI) m/z calcd for (M+H)⁺ C₂₇H₃₃N₂O₄ 449.2455. found 449.2440 (3.3 ppm).

### Compound 5i: (S)-benzyl 3-((S)-3-(tert-butoxy)-2-((S)-sec-butyl)-5-oxo-2,5-dihydro-1H-pyrrol-1-yl)pyrrolidine-1-carboxylate

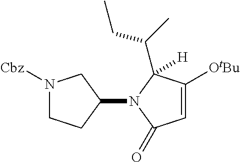

Procedure: As described for compound 5f (in this case the reaction is slower due to steric hindrance and was complete after 24 h; slightly higher yields were obtained by carrying out the reaction in dioxane at 100° C.). Column chromatography using 5% acetone in dichloromethane as eluent afforded the product in 60% yield. [α]²°+42.1 (c 0.8, MeOH)

¹H-NMR (300 MHz, CDCl₃) δ 7.30-7.18 (m, 5H), 5.09 (s, 2H), 4.96 (s, 1H), 4.02-3.92 (m, 1H), 3.77 (d, J=2.4 Hz, 1H), 3.70-3.52 (m, 3H), 3.32-3.21 (m, 1H), 2.54-2.33 (m, 1H), 2.02-1.90 (m, 1H), 1.80-1.69 (m, 1H), 1.58-1.40 (m, 1H), 1.39 (s, 9H), 1.24-1.18 (m, 1H), 0.94-0.84 (m, 3H), 0.71-0.6 (m, 3H); HRMS (ESI) m/z calcd for (M+H)⁺ C₂₄H₃₄N₂O₄ 415.2597. found 415.2580 (4.1 ppm).

### Compound 6f: (S)-5-benzyl-4-(tert-butoxy)-1-((S)-pyrrolidin-3-yl)-1H-pyrrol-2(5H)-one

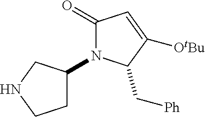 [SMILES: CC(C)(C)OC1=CC(=O)N([C@H]2CCNC2)[C@H]1Cc1ccccc1]

Procedure: To a stirred solution of 5f (410 mg, 0.91 mmol) in methanol (9 mL) under nitrogen was carefully added 10 wt % Pd/C (195 mg, 0.2 equiv Pd) at 25° C. The reaction was evacuated, refilled with N₂, and placed under an atmosphere of H₂ (1 atm, balloon) for 12 h. The reaction mixture was purged with N₂, and filtered over a pad of celite under a gentle vacuum (SAFETY NOTE: Do not let the pad run dry). The celite pad was washed with methanol (2×25 mL), and the combined filtrates were concentrated. Dichloromethane (2×5 mL) was added and the residue was re-concentrated to remove residual methanol. The residue was placed under a high vacuum (<5 mm Hg) for 2 h to afford the product, which was crystallized from MeCN in 88% yield.

¹H-NMR (300 MHz, CDCl₃) δ 7.32-7.24 (m, 3H), 7.12-7.06 (m, 2H), 5.17 (app s, 1H), 4.07 (t, J=5.4 Hz, 1H), 3.77-3.69 (m, 1H), 3.57-3.46 (m, 1H), 3.34-3.28 (m, 3H), 3.18 (dd, J=14.6, 4.7 Hz, 1H), 2.91 (dd, J=14.4, 6.0 Hz, 1H), 2.38-2.26 (m, 1H), 2.02-1.90 (m, 1H), 1.48 (s, 9H); HRMS (ESI) m/z calcd for (M+H) C₁₉H₂₇N₂O₂ 315.2073. found 315.2062 (3.5 ppm).

### Compound 6i: (S)-4-(tert-butoxy)-5-((S)-sec-butyl)-1-((S)-pyrrolidin-3-yl)-1H-pyrrol-2(5H)-one

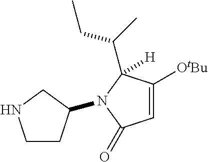

Procedure: To a stirred solution of the starting material 5c (180 mg, 0.43 mmol) in methanol (5 mL) under nitrogen was carefully added 10 wt % Pd/C (92 mg, 0.2 equiv Pd) at 25° C. The reaction was evacuated, refilled with N₂, and placed under an atmosphere of H₂ (1 atm, balloon) for 12 h. The reaction mixture was purged with N₂, and filtered over a pad of celite under a gentle vacuum (SAFETY NOTE: Do not let the pad run dry). The celite pad was washed with methanol (2×15 mL), and the combined filtrates were concentrated. The residue was purified by column chromatography (SiO₂, 5% MeOH/CH₂Cl₂→5% MeOH/CH₂Cl₂ containing 1% Et₃N) to afford the product in 96% yield.

¹H-NMR (300 MHz, CDCl₃) δ 4.92 (app s, 1H), 3.80 (m, 1H), 3.76 (d, J=2.4 Hz, 1H), 3.26-3.18 (m, 2H), 2.94 (dd, J=11.9 and 8.3 Hz, 1H), 2.76-2.72 (m, 1H), 2.04-1.96 (m, 2H), 1.84-1.74 (m, 1H), 1.54-1.41 (m, 2H), 1.39 (s, 9H), 0.92 (t, J=7.5 Hz, 3H), 0.72 (d, J=6.9 Hz, 3H); HRMS (ESI) m/z calcd for C₁₄H₂₉N₂O₂ (M+H)⁺ 281.2229. found 281.2222 (2.5 ppm).

### Compound 6a: (S)-4-(tert-butoxy)-5-methyl-1-((S)-pyrrolidin-3-yl)-1H-pyrrol-2(5H)-one

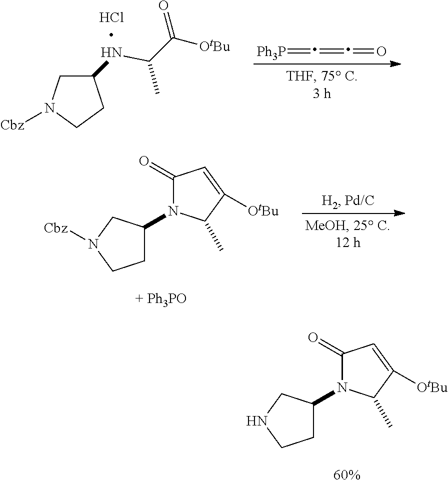

Procedure: The same procedure for cyclization to obtain 5f was used here. The reaction was complete in 3 h. Upon cooling, the THF was removed in vacuo and the residue was loaded onto a short SiO₂ column. Elution with 5% EtOAc/CH₂Cl₂ (to remove traces of unreacted starting material) followed by 100% EtOAc, afforded a mixture of the cyclized product and triphenylphosphine oxide. The mixture was directly utilized in the next step.

To a stirred solution of the above mixture (1.4 g) in methanol (30 mL) under nitrogen was carefully added 10 wt % Pd/C (729 mg, 0.2 equiv Pd) at 25° C. The reaction was evacuated, refilled with N₂, and placed under an atmosphere of H₂ (1 atm, balloon) for 12 h. The reaction mixture was purged with N₂, and filtered over a pad of celite under a gentle vacuum (SAFETY NOTE: Do not let the pad run dry). The celite pad was washed with methanol (2×40 mL), and the combined filtrates were concentrated. The residue was purified by column chromatography (SiO₂, 5% MeOH/CH₂Cl₂→5% MeOH/CH₂Cl₂ containing 1% Et₃N→10% MeOH/CH₂Cl₂ containing 1% TEA) to afford the product (490 mg, 60% yield). Note: Concentration of the rich fractions was performed by first adding toluene (˜10 mL/100 mL of eluent) to protect from epimerization by Et₃N. ¹H-NMR (300 MHz, CDCl₃) δ 4.83 (app s, 1H), 4.00-3.84 (m, 1H), 3.73 (q, J=6.6 Hz, 1H), 3.58 (br s, 1H), 3.21-3.09 (m, 1H), 3.05 (dd, J=11.6, 5.0 Hz, 1H), 2.90 (dd, J=11.7, 7.7 Hz, 1H), 2.77-2.61 (m, 1H), 2.06-1.91 (m, 1H), 1.90-1.74 (m, 1H), 1.32 (s, 9H), 1.20 (d, J=6.6 Hz, 3H); HRMS (ESI) m/z calcd for C₁₃H₂₃N₂O₂ (M+H)⁺ 239.1760. found 239.1752 (3.3 ppm).

### Compound 6l: (S)-4-(tert-butoxy)-5-isobutyl-1-((S)-pyrrolidin-3-yl)-1H-pyrrol-2(5H)-one

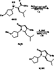

Procedure: As described for 6a.

¹H-NMR (300 MHz, CDCl₃) δ 4.91 (s, 1H), 3.87-3.71 (m, 2H), 3.30-3.14 (m, 2H), 2.90 (dd, J=12.0, 8.1 Hz, 1H), 2.75-2.56 (m, 2H), 2.08-1.92 (m, 1H), 1.92-1.81 (m, 1H), 1.81-1.68 (m, 1H), 1.62-1.52 (m, 2H), 1.39 (s, 9H), 0.88 (d, J=6.6 Hz, 3H); HRMS (ESI) m/z calcd for C₁₆H₂₉N₂O₂ (M+H)⁺ 281.2229. found 281.2222 (2.5 ppm).

### Compound 7m: (S)-5-(2-(methylthio)ethyl)pyrrolidine-2,4-dione

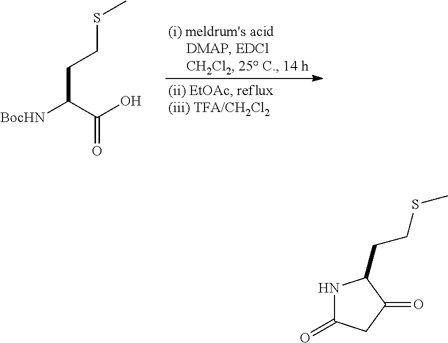

Procedure: A modified literature procedure was used.³ To a stirred solution of meldrum's acid (476 mg, 3.3 mmol, 1.1 equiv) and DMAP (550 mg, 4.5 mmol, 1.5 equiv) at 0° C. in dichloromethane (30 mL) was added N-Boc-Met-OH (748 mg, 3.0 mmol, 1.0 equiv) in one portion. EDCI (1.2 g, 7.2 mmol, 2.4 equiv) was added in one portion and the reaction mixture was stirred at 25° C. for 14 h. The yellow reaction mixture was transferred to a separatory funnel and diluted with ACS reagent grade EtOAc (80 mL) and washed with cold 5% KHSO₄ (3×50 mL) and brine (75 mL). The organic layer was dried over MgSO₄ and filtered. The filtrate was refluxed for 30 min under N₂. Upon concentration, the residue was dissolved in dichloromethane (8 mL) and cooled to 0° C. TFA (8 mL) was added and the reaction was stirred for 30 min. Toluene (25 mL) was added and the solution was concentrated. Residual TFA was azeotroped 3 times with toluene (25 mL ea) and the residue was placed under high vacuum for 3 h. A small portion of dichloromethane was added to obtain a concentrated solution and few drops of hexanes were added to afford crystals at −20° C. The crystals were collected by filtration and washed with cold hexanes to obtain the pure product (333 mg, 64%). NOTE: The crystals are stable at room temperature for several weeks but assume a yellow coloration. It is best stored at −20° C. under N₂. [α]²°+1.9 (c 1.0, MeOH); ¹H-NMR (300 MHz, CDCl₃) δ 8.38 (s, 1H), 4.04 (t, J=5.5 Hz, 1H), 3.02 (d, J=22.1 Hz, 1H), 2.92 (d, J=22.1 Hz, 1H), 2.49 (t, J=6.7 Hz, 2H), 2.02-1.88 (m, 2H), 1.91 (s, 3H); MS (ESI) m/z calcd for (M+H)⁺ C₇H₁₂NO₂S 174.05. found 174.07.

### Compound 7t′: (S)-5-((R)-1-(benzyloxy)ethyl)pyrrolidine-2,4-dione

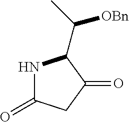 [SMILES: C[C@@H](OCc1ccccc1)[C@@H]1NC(=O)CC1=O]

Procedure: As per the procedure used for 7m. [α]²¹-53.1 (c 0.5, MeOH); ¹H-NMR (300 MHz, CDCl₃) δ 8.1 (br s, 1H), 7.36-7.25 (m, 3H), 7.25-7.18 (m, 2H), 4.56 (d, J=11.7 Hz, 1H), 4.33 (d, J=11.7 Hz, 1H), 3.98-3.83 (m, 2H), 2.99 (d, J=21.9 Hz, 1H), 2.89 (d, J=21.9 Hz, 1H), 1.30 (d, J=6.0 Hz, 3H); HRMS (ESI) m/z calcd for C₁₃H₁₆NO₃ (M+H)⁺ 234.1130. found 234.1139 (3.8 ppm).

### Compound 7d′: (S)-benzyl 2-(3,5-dioxopyrrolidin-2-yl)acetate

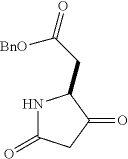 [SMILES: O=C1CC(=O)[C@H](CC(=O)OCc2ccccc2)N1]

Procedure: As per the procedure used for 7m. [α]²¹−33.4 (c 0.5, MeOH); ¹H-NMR (300 MHz, CDCl₃) δ 7.44-7.28 (m, 5H), 5.15 (s, 2H), 4.30-4.19 (m, 1H), 3.17-2.98 (m, 2H), 2.98-2.78 (m, 2H); HRMS (ESI) m/z calcd for C₁₃H₁₄NO₄ (M+H)⁺ 248.0922. found 248.0926 (1.3 ppm).

### Compound 7a

### (S)-5-methylpyrrolidine-2,4-dione

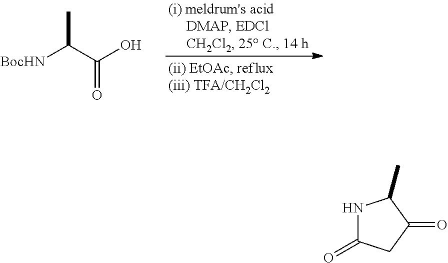

Procedure: As described above for (S)-5-(2-(methylthio)ethyl)pyrrolidine-2,4-dione, 7m. The product (white solid) was obtained by addition of dry ether to the crude residue (brown oil), and stirring for 14 h (890 mg, 92%). The spectra match (¹H and ¹³C-NMR) the reported spectra.³

### Compound 7f

### (S)-5-benzylpyrrolidine-2,4-dione

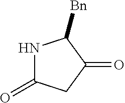 [SMILES: O=C1CC(=O)[C@H](Cc2ccccc2)N1]

Procedure: As described above for (S)-5-(2-(methylthio)ethyl)pyrrolidine-2,4-dione, 7m. The product (white solid) was obtained by addition of dry ether and hexanes to the crude residue (brown oil), and stirring for 2 h (75%). The spectra match (¹H and ¹³C-NMR) the reported spectra.³

### Compound 71

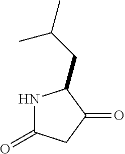

Procedure: As described above for (S)-5-(2-(methylthio)ethyl)pyrrolidine-2,4-dione, 7m. The product (white solid) was obtained by addition of dry ether and hexanes to the crude residue (brown oil), and stirring for 2 h (70%). The spectra match (¹H and ¹³C-NMR) the reported spectra.

### Compound 2af-^{t}Bu: (S)-5-benzyl-4-(tert-butoxy)-1-((S)-1-((S)-2-methyl-5-oxo-2,5-dihydro-1H-pyrrol-3-yl)pyrrolidin-3-yl)-1H-pyrrol-2(5H)-one

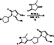

Procedure: To a stirred solution of the amine (1 mmol) and tetramic acid (1.2 mmol) in ⁱPrOH (0.1 M) was added trimethylorthoformate (1.5 mmol, 164 μL) at 25° C. under Argon. The reaction mixture was stirred for 5 h and concentrated at 25° C. The residue was purified by flash chromatography (4-5% MeOH/CH₂Cl₂) to afford the product in 68% yield. [α]²°+56.1 (c 0.7, MeOH); ¹H-NMR (300 MHz, CDCl₃) δ 7.31-7.20 (m, 3H), 7.17-7.09 (m, 2H), 5.23 (br s, 1H), 4.90 (app s, 1H), 4.48 (app s, 1H), 4.25-4.08 (m, 2H), 4.07-0.88 (m, 1H), 3.70 (t, J=8.9 Hz, 1H), 3.49-3.32 (m, 2H), 3.30-3.18 (m, 1H), 3.12 (dd, J=14.4, 4.6 Hz, 1H), 3.00 (dd, J=14.5, 4.7 Hz, 1H), 2.52 (p, J=10.2 Hz, 1H), 2.24-2.05 (m, 1H), 1.37 (s, 9H); HRMS (ESI) m/z calcd for C₂₄H₃₂N₃O₃ (M+H)⁺ 410.2444. found 410.2462 (4.4 ppm).

### Compound 2mi-^{t}Bu: (S)-4-(tert-butoxy)-5-((S)-sec-butyl)-1-((S)-1-((S)-2-(2-(methylthio)ethyl)-5-oxo-2,5-dihydro-1H-pyrrol-3-yl)pyrrolidin-3-yl)-1H-pyrrol-2(5H)-one

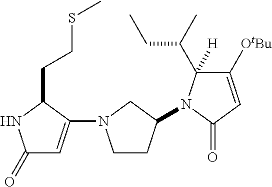 [SMILES: CC[C@H](C)[C@H]1C(OC(C)(C)C)=CC(=O)N1[C@H]1CCN(C2=CC(=O)N[C@H]2CCSC)C1]

Procedure: As described for compound 2af-^{t}Bu. 71% yield. ¹H-NMR (300 MHz, CDCl₃) δ 5.96 (br s, 1H), 5.00 (app s, 1H), 4.55 (app s, 1H), 4.32 (d, J=7.2 Hz, 1H), 4.09-3.94 (m, 1H), 3.86 (d, J=2.1 Hz, 1H), 3.84-3.70 (m, 1H), 3.49-3.34 (m, 2H), 3.32-3.18 (m, 1H), 2.72-2.59 (m, 1H), 2.55 (t, J=7.2 Hz, 2H), 2.17-2.05 (m, 2H), 2.09 (s, 3H), 1.89-1.72 (m, 2H), 1.63-1.47 (m, 2H), 1.43 (s, 9H), 0.97 (app t, J=7.4 Hz, 3H), 0.76 (d, J=6.9 Hz, 3H); HRMS (ESI) m/z calcd for (M+H)⁺ 436.2634. found 436.2624 (2.3 ppm).

### Compound 2aa-^{t}Bu: (S)-4-(tert-butoxy)-5-methyl-1-((S)-1-((S)-2-methyl-5-oxo-2,5-dihydro-1H-pyrrol-3-yl)pyrrolidin-3-yl)-1H-pyrrol-2(5H)-one

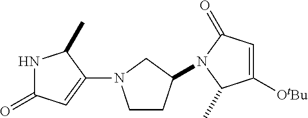 [SMILES: C[C@@H]1NC(=O)C=C1N1CC[C@H](N2C(=O)C=C(OC(C)(C)C)[C@@H]2C)C1]

Procedure: As described for compound 2af-^{t}Bu. 65% yield. [α]²°+12.5 (c 1.0, MeOH); ¹H-NMR (300 MHz, CDCl₃) δ 5.55 (br s, 1H), 4.98 (app s, 1H), 4.49 (app s, 1H), 4.34-4.18 (m, 2H), 3.91-3.82 (m, 1H), 3.64-3.52 (m, 1H), 3.51-3.36 (m, 2H), 3.34-3.25 (m, 1H), 2.54-2.42 (m, 1H), 2.26-2.14 (m, 1H), 1.43 (s, 9H), 1.38-1.30 (m, 6H); HRMS (ESI) m/z calcd for C₁₈H₂₇N₃O₃ 334.2131 (M+H)⁺. found 334.2136 (1.5 ppm).

### Compound 2la-^{t}Bu: (5S)-4-(tert-butoxy)-1-((3S)-1-(2-isobutyl-5-oxo-2,5-dihydro-1H-pyrrol-3-yl)pyrrolidin-3-yl)-5-methyl-1H-pyrrol-2(5H)-one

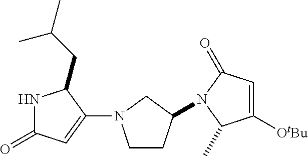 [SMILES: CC(C)C[C@@H]1NC(=O)C=C1N1CC[C@H](N2C(=O)C=C(OC(C)(C)C)[C@@H]2C)C1]

¹H-NMR (300 MHz, CDCl₃) δ 5.52 (br s, 1H), 4.99 (s, 1H), 4.53 (d, J=1.5 Hz, 1H), 4.35-4.20 (m, 1H), 4.19-4.12 (m, 1H), 3.87 (q, J=6.6 Hz, 1H), 3.63-3.51 (m, 1H), 3.47-3.35 (m, 2H), 3.35-3.21 (m, 1H), 2.57-2.39 (m, 1H), 2.26-2.13 (m, 1H), 1.93-1.79 (m, 1H), 1.78-1.67 (m, 1H), 1.66-1.56 (m, 1H), 1.44 (s, 9H), 1.34 (d, J=6.6 Hz, 3H); HRMS (ESI) m/z calcd for C₂₁H₃₄N₃O₃ (M+H)⁺ 376.2600. found 376.2588 (3.2 ppm).

### Compound 2t′i-^{t}Bu: (S)-1-((S)-1-((R)-2-((R)-1-(benzyloxy)ethyl)-5-oxo-2,5-dihydro-1H-pyrrol-3-yl)pyrrolidin-3-yl)-4-(tert-butoxy)-5-((S)-sec-butyl)-1H-pyrrol-2(5H)-one

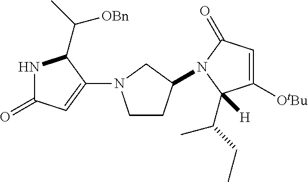 [SMILES: CC[C@H](C)[C@H]1C(OC(C)(C)C)=CC(=O)N1[C@H]1CCN(C2=CC(=O)N[C@H]2C(C)OCc2ccccc2)C1]

Procedure: As described for compound 2af-^{t}Bu. 64% yield. [α]²¹+27.9 (c 1.7, MeOH); ¹H-NMR (300 MHz, CDCl₃) δ 7.35-7.15 (m, 5H), 6.90 (br s, 1H), 4.96 (s, 1H), 4.63-4.50 (m, 2H), 4.42 (d, J=12.0 Hz, 1H), 4.23 (d, J=1.8 Hz, 1H), 4.10-3.93 (m, 1H), 3.87-3.77 (m, 1H), 3.77-3.69 (m, 1H), 3.64-3.51 (m, 1H), 3.39-3.22 (m, 2H), 3.22-3.10 (m, 1H), 2.50-2.31 (m, 1H), 2.10-1.92 (m, 1H), 1.81-1.64 (m, 1H), 1.52-1.33 (m, 11H), 1.16 (d, J=6.0 Hz, 3H), 0.91 (t, J=7.4 Hz, 3H), 0.66 (d, J=6.6 Hz, 3H); HRMS (ESI) m/z calcd for C₂₉H₄₂N₃O₄ (M+H)⁺ 496.3175. found 496.3158 (3.5 ppm).

### Compound 2d′i-^{t}Bu: benzyl 2-((S)-3-((S)-3-((S)-3-(tert-butoxy)-2-((S)-sec-butyl)-5-oxo-2,5-dihydro-1H-pyrrol-1-yl)pyrrolidin-1-yl)-5-oxo-2,5-dihydro-1H-pyrrol-2-yl)acetate

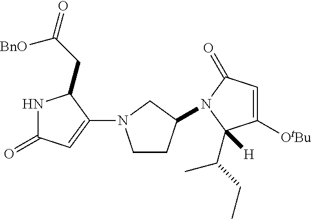 [SMILES: CC[C@H](C)[C@H]1C(OC(C)(C)C)=CC(=O)N1[C@H]1CCN(C2=CC(=O)N[C@H]2CC(=O)OCc2ccccc2)C1]

Procedure: As described for compound 2af-^{t}Bu; the reaction was run for 8 h to afford 2d′i-^{t}Bu in 55% yield. ¹H-NMR (300 MHz, CDCl₃) δ 7.35-7.23 (m, 5H), 5.67 (br s, 1H), 5.08 (s, 2H), 4.93 (s, 1H), 4.49 (s, 1H), 4.43 (dd, J=10.8, 2.1 Hz, 1H), 3.97-3.83 (m, 1H), 3.78 (d, J=2.4 Hz, 1H), 3.79-3.70 (m, 1H), 3.41-3.25 (m, 2H), 3.25-3.08 (m, 1H), 2.92 (dd, J=17.0, 2.6 Hz, 1H), 2.68-2.49 (m, 1H), 2.36 (dd, J=16.9, 11.0 Hz, 1H), 2.11-1.93 (m, 1H), 1.81-1.64 (m, 1H), 1.60-1.39 (m, 2H), 1.37 (s, 9H), 0.91 (t, J=7.4 Hz, 3H), 0.70 (d, J=6.9 Hz, 3H); HRMS (ESI) m/z calcd for C₂₉H₄₀N₃O₅ (M+H)⁺ 510.2967. found 510.2960 (1.6 ppm).

### Compound 2t′i-H: (S)-1-((S)-1-((R)-2-((R)-1-(benzyloxy)ethyl)-5-oxo-2,5-dihydro-1H-pyrrol-3-yl)pyrrolidin-3-yl)-4-(tert-butoxy)-5-((S)-sec-butyl)-1H-pyrrol-2(5H)-one

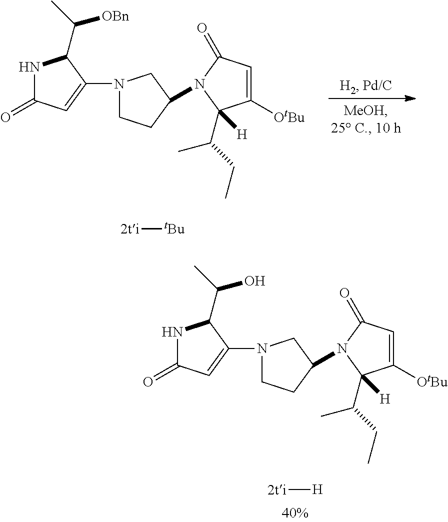

Procedure: Compound 1ec (0.22 mmol) in MeOH (2 mL) was subject to hydrogenloysis using 10% Pd/C (47 mg, 0.2 eq Pd) for 10 h. The reaction was purges with N₂ for a few minutes and filtered over Celite. The filtrate was concentrated and purified by flash chromatography (5-7% MeOH/CH₂Cl₂) to afford the product in 40% yield. [α]²¹−2.2 (c 1.2, MeOH); ¹H-NMR (300 MHz, CDCl₃) δ 4.93 (s, 1H), 4.55 (s, 1H), 4.13-3.92 (m, 3H), 3.80 (d, J=2.7 Hz, 1H), 3.78-3.66 (m, 1H), 3.45-3.34 (m, 2H), 3.34-3.18 (m, 1H), 2.63-2.42 (m, 1H), 2.14-1.99 (m, 1H), 1.83-1.69 (m, 1H), 1.61-1.41 (m, 2H), 1.37 (s, 9H), 1.25 (d, J=9.0 Hz, 3H), 0.91 (t, J=7.4 Hz, 3H), 0.70 (d, J=6.6 Hz, 3H); HRMS (ESI) m/z calcd for C₂₂H₃₆N₃O₄ (M+H)⁺ 406.2705. found 406.2692 (3.4 ppm).

### 2ti-^{t}Bu. (S)-1-((S)-1-((R)-2-((R)-1-(benzyloxy)ethyl)-5-oxo-2,5-dihydro-1H-pyrrol-3-yl)pyrrolidin-3-yl)-4-(tert-butoxy)-5-((S)-sec-butyl)-1H-pyrrol-2(5H)-one

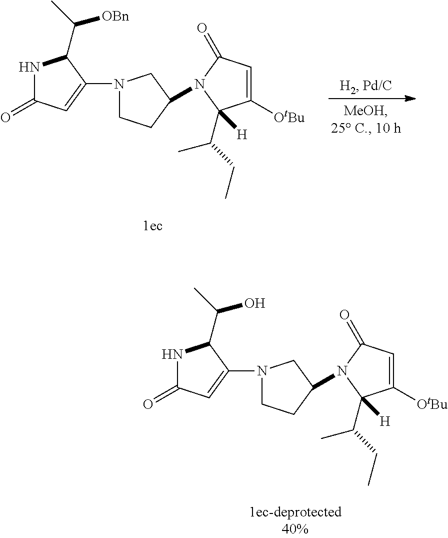 [SMILES: CC[C@H](C)[C@H]1C(OC(C)(C)C)=CC(=O)N1[C@H]1CCN(C2=CC(=O)N[C@H]2[C@@H](C)OCc2ccccc2)C1 ; CC[C@H](C)[C@H]1C(OC(C)(C)C)=CC(=O)N1[C@H]1CCN(C2=CC(=O)N[C@H]2[C@@H](C)O)C1]

Procedure: Compound 1ec (0.22 mmol) in MeOH (2 mL) was subject to hydrogenloysis using 10% Pd/C (47 mg, 0.2 eq Pd) for 10 h. The reaction was purges with N₂ for a few minutes and filtered over Celite. The filtrate was concentrated and purified by flash chromatography (5-7% MeOH/CH₂Cl₂) to afford the product in 40% yield.

Physical state: white solid

[α]²¹−2.2 (c 1.2, MeOH)

¹H-NMR (300 MHz, CDCl₃) δ 4.93 (s, 1H), 4.55 (s, 1H), 4.13-3.92 (m, 3H), 3.80 (d, J=2.7 Hz, 1H), 3.78-3.66 (m, 1H), 3.45-3.34 (m, 2H), 3.34-3.18 (m, 1H), 2.63-2.42 (m, 1H), 2.14-1.99 (m, 1H), 1.83-1.69 (m, 1H), 1.61-1.41 (m, 2H), 1.37 (s, 9H), 1.25 (d, J=9.0 Hz, 3H), 0.91 (t, J=7.4 Hz, 3H), 0.70 (d, J=6.6 Hz, 3H)

¹³C-NMR (75 MHz, CDCl₃) δ 177.6, 173.4, 170.5, 164.3, 97.4, 90.0, 82.0, 65.8, 65.4, 62.9, 52.4, 50.3, 47.9, 36.5, 27.8, 27.4, 26.0, 21.0, 12.6, 12.4

IR (film, cm⁻¹) 3302 (br), 2968, 2874, 1653, 1595, 1396, 1375, 1253, 1167, 779, 735

HRMS (ESI) m/z calcd for C₂₂H₃₆N₃O₄ (M+H)⁺ 406.2705. found 406.2692 (3.4 ppm).

### Compound 2ff-^{t}Bu: (S)-5-benzyl-1-((S)-1-((S)-2-benzyl-5-oxo-2,5-dihydro-1H-pyrrol-3-yl)pyrrolidin-3-yl)-4-(tert-butoxy)-1H-pyrrol-2(5H)-one

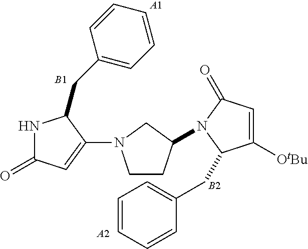

Procedure: As described for compound 2af-^{t}Bu. 55% yield. [α]²⁰-1.5 (c 1.0, MeOH); ¹H-NMR (300 MHz, CDCl₃) δ 7.37-7.22 (m, 6H), 7.21-7.12 (m, 4H), 5.07 (br s, 1H), 4.92 (app s, 1H), 4.51 (d, J=1.5 Hz, 1H), 4.24 (dd, J=9.6, 2.7 Hz, 1H), 4.17 (t, J=4.9 Hz, 1H), 4.10-3.94 (m, 1H), 3.89-3.73 (m, 1H), 3.54-3.36 (m, 2H), 3.34-3.26 (m, 1H), 3.23 (dd, J=13.8, 3.0 Hz, 1H), 3.14 (dd, J=14.6, 5.0 Hz, 1H), 3.03 (dd, J=14.8, 5.0 Hz, 1H), 2.68-2.45 (m, 2H), 2.25-2.09 (m, 1H), 1.38 (s, 9H); HRMS (ESI) m/z calcd for (M+H)⁺ C₃₀H₃₆N₃O₃ 486.2756; 486.2778 found (4.4 ppm).

### Compound 2fl-^{t}Bu: (5S)-1-((3S)-1-(2-benzyl-5-oxo-2,5-dihydro-1H-pyrrol-3-yl)pyrrolidin-3-yl)-4-(tert-butoxy)-5-isobutyl-1H-pyrrol-2(5H)-one

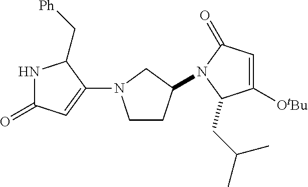 [SMILES: CC(C)C[C@H]1C(OC(C)(C)C)=CC(=O)N1[C@H]1CCN(C2=CC(=O)NC2Cc2ccccc2)C1]

Procedure: As described for compound 2af-^{t}Bu. 74% yield. ¹H-NMR (300 MHz, CDCl₃) δ 7.35-7.08 (m, 5H), 5.17 (br s, 1H), 4.98 (s, 1H), 4.51 (d, J=1.5 Hz, 1H), 4.30 (dd, J=9.6, 2.7 Hz, 1H), 4.21-4.00 (m, 1H), 3.90 (dd, J=6.3, 3.9 Hz, 1H), 3.86-3.72 (m, 1H), 3.62-3.40 (m, 2H), 3.40-3.29 (m, 1H), 3.25 (dd, J=13.6, 2.9 Hz, 1H), 2.66-2.47 (m, 2H), 2.24-2.10 (m, 1H), 1.89-1.74 (m, 1H), 1.68-1.55 (m, 2H), 1.43 (s, 9H), 0.93 (d, J=6.6 Hz, 3H); HRMS (ESI) m/z calcd for C₂₇H₃₈N₃O₃ (M+H)⁺ 452.2913. found 452.2906 (1.5 ppm).

### Compound 2fl-H: (3′S,5S)-1′-(2-benzyl-5-oxo-2,5-dihydro-1H-pyrrol-3-yl)-5-isobutyl-[1,3′-bipyrrolidine]-2,4-dione

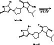

Procedure: As described below for 2aa-H.

¹H-NMR (300 MHz, CDCl₃) δ 7.36-7.17 (m, 5H), 5.54 (br s, 1H), 4.55 (s, 1H), 4.39-4.33 (m, 1H), 4.28-4.12 (m, 1H), 4.02-3.94 (m, 1H), 3.84-3.72 (m, 1H), 3.71-3.61 (m, 1H), 3.60-3.48 (m, 1H), 3.30-3.27 (m, 2H), 3.16-2.98 (m, 2H), 2.69-2.49 (m, 2H), 2.39-2.26 (m, 1H), 1.98-1.81 (m, 1H), 1.74-1.61 (m, 2H), 0.99 (d, J=2.1 Hz, 3H), 0.97 (d, J=2.1 Hz, 3H).

### Compound 1aaa-^{t}Bu: (S)-4-(tert-butoxy)-5-methyl-1-((S)-1-((S)-2-methyl-1-((S)-1-((S)-2-methyl-5-oxo-2,5-dihydro-1H-pyrrol3-yl)pyrrolidin-3-yl)-5-oxo-2,5-dihydro-1H-pyrrol-3-yl)pyrrolidin-3-yl)-1H-pyrrol-2(5H)-one

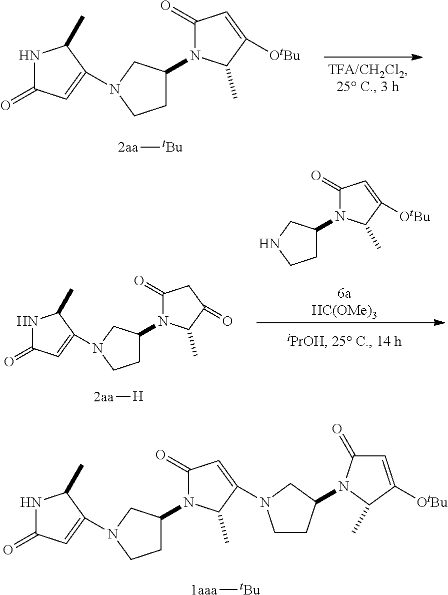

Procedure: 2aa-^{t}Bu (0.8 mmol) was treated with TFA/CH₂Cl₂ (1:1, 1.5 mL) at 25° C. The reaction was stirred in a vented teflon-capped flask until complete disappearance of starting material (monitored by NMR spectroscopy, ˜3 h). Toluene (5 mL) was added and the reaction mixture was concentrated in vacuo. Toluene (2×5 mL) was used to azeotrope residual TFA, and the residue was stirred with dry Et₂O (10 mL). After 2 h, the ether was decanted and the residue was dried under high vacuum to afford 2aa-H as a white solid in quantitative yield. The tetramic acid 2aa-H (0.8 mmol) and amine 6a (0.8 mmol) were stirred in ⁱPrOH (8 mL) under Argon. Trimethylorthoformate (1.2 mmol, 131 μL) was added and the reaction was allowed to proceed for 14 h. The reaction mixture was concentrated at 25° C. to obtain a yellow foam. The product was purified by flash chromatography (4-7% MeOH/CH₂Cl₂) to afford 1aaa-^{t}Bu in 45% yield. [α]²°+17.8 (c 0.9, MeOH); ¹H-NMR (300 MHz, CDCl₃) δ 5.27 (br s, 1H), 4.98 (app s, 1H), 4.53 (app s, 1H), 4.49 (app s, 1H), 4.30-4.14 (m, 3H), 4.13-4.03 (m, 1H), 3.92-3.81 (m, 1H), 3.66-3.54 (m, 2H), 3.53-3.35 (m, 4H), 3.35-3.17 (m, 2H), 2.60-2.38 (m, 2H), 2.28-2.09 (m, 2H), 1.44 (s, 9H), 1.42-1.32 (m, 9H); HRMS (MALDI) m/z calcd for (M+H)⁺ C₂₇H₄₀N₅O₄ 498.3080. found 498.3079 (1.0 ppm).

### 2ll-^{t}Bu. (S)-4-(Tert-butoxy)-5-isobutyl-1-((S)-1-((S)-2-isobutyl-5-oxo-2,5-dihydro-1H-pyrrol-3-yl)pyrrolidin-3-yl)-1H-pyrrol-2(5H)-one

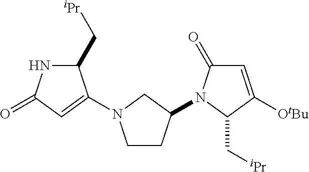 [SMILES: CC(C)C[C@@H]1NC(=O)C=C1N1CC[C@H](N2C(=O)C=C(OC(C)(C)C)[C@@H]2CC(C)C)C1]

Procedure: As per the general procedure for the one-pot coupling reaction.

Physical state: White solid (54%).

¹H-NMR (300 MHz, CDCl₃) δ 5.78 (br s, 1H), 4.97 (s, 1H), 4.51 (d, J=1.5 Hz, 1H), 4.21-3.97 (m, 2H), 3.87 (dd, J=6.3, 3.8 Hz, 1H), 3.79-3.64 (m, 1H), 3.46-3.33 (m, 2H), 3.32-3.14 (m, 1H), 2.65-2.43 (m, 1H), 2.23-2.06 (m, 2H), 1.87-1.67 (m, 2H), 1.67-1.53 (m, 3H), 1.42 (s, 9H), 0.99-0.83 (m, 12H)

¹³C-NMR (75 MHz, CDCl₃) δ 176.3, 173.3, 171.8, 166.8, 96.1, 88.3, 82.0, 61.3, 55.8, 52.5, 49.8, 47.4, 42.1, 39.7, 27.4, 25.8, 24.1, 23.9, 23.7, 23.0, 21.3

IR (film, cm⁻¹) 3211 (br), 2955, 1661, 1651, 1601, 1472, 1371, 1341, 1268, 1167, 731

HRMS (ESI) m/z calcd for C₂₄H₄₀N₃O₃ (M+H)⁺ 418.3070. found 418.3067 (0.6 ppm).

### 2al-^{t}Bu. (S)-4-(Tert-butoxy)-5-isobutyl-1-((S)-1-((S)-2-methyl-5-oxo-2,5-dihydro-1H-pyrrol-3-yl)pyrrolidin-3-yl)-1H-pyrrol-2(5H)-one

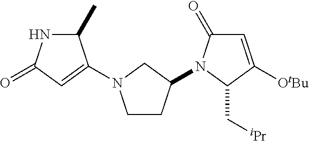 [SMILES: CC(C)C[C@H]1C(OC(C)(C)C)=CC(=O)N1[C@H]1CCN(C2=CC(=O)N[C@H]2C)C1]

Procedure: As per the general procedure for the one-pot coupling reaction.

Physical state: White solid (48%).

¹H-NMR (300 MHz, CDCl₃) δ 5.72 (br s, 1H), 4.97 (s, 1H), 4.48 (d, J=1.2 Hz, 1H), 4.20 (q, J=6.7 Hz, 1H), 4.14-3.99 (m, 1H), 3.87 (dd, J=6.4, 3.8 Hz, 1H), 3.77-3.62 (m, 1H), 3.51-3.33 (m, 2H), 3.33-3.16 (m, 1H), 2.62-2.43 (m, 1H), 2.19-2.06 (m, 1H), 1.87-1.71 (m, 1H), 1.65-1.53 (m, 2H), 1.42 (s, 9H), 1.35 (d, J=6.6 Hz, 3H), 0.90 (d, J=6.3 Hz, 6H)

¹³C-NMR (75 MHz, CDCl₃) δ 176.2, 173.3, 171.9, 167.3, 96.1, 87.7, 82.0, 61.2, 52.9, 52.5, 49.8, 47.5, 39.7, 27.7, 27.4, 24.1, 23.9, 23.0, 19.0

IR (film, cm⁻¹) 3304 (br), 2976, 2871, 1653, 1601, 1397, 1339, 1258, 1167, 731

HRMS (ESI) m/z calcd for C₂₁H₃₄N₃O₃ (M+H)⁺ 376.2600. found 376.2610 (2.6 ppm).

### 2t′l-^{t}Bu. (5S)-1-((3S)-1-(2-((R)-1-(Benzyloxy)ethyl)-5-oxo-2,5-dihydro-1H-pyrrol-3-yl)pyrrolidin-3-yl)-4-(tert-butoxy)-5-isobutyl-1H-pyrrol-2(5H)-one

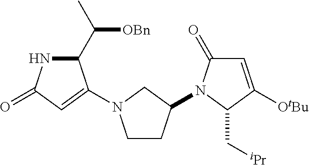 [SMILES: CC(C)C[C@H]1C(OC(C)(C)C)=CC(=O)N1[C@H]1CCN(C2=CC(=O)N[C@H]2[C@@H](C)OCc2ccccc2)C1]

Procedure: As per the general procedure for the one-pot coupling reaction.

Physical state: White solid (53%).

¹H-NMR (300 MHz, CDCl₃) δ 7.36-7.21 (m, 5H), 5.78 (br s, 1H), 4.98 (s, 1H), 4.64-4.55 (m, 2H), 4.44 (d, J=11.7 Hz, 1H), 4.21 (d, J=1.5 Hz, 1H), 4.19-4.07 (m, 1H), 3.83-3.74 (m, 1H), 3.52-3.27 (m, 3H), 3.24-3.11 (m, 1H), 2.41-2.25 (m, 1H), 2.14-2.05 (m, 1H), 1.99-1.86 (m, 1H), 1.82-1.69 (m, 1H), 1.63-1.51 (m, 2H), 1.44 (s, 9H), 1.19 (d, J=6.3 Hz, 3H), 0.89 (d, J=6.3 Hz, 6H)

¹³C-NMR (75 MHz, CDCl₃) δ 176.6, 173.3, 171.8, 163.8, 138.2, 128.3, 127.8, 127.6, 96.0, 90.3, 81.9, 73.8, 71.0, 61.3, 60.8, 51.9, 50.8, 48.1, 39.7, 28.1, 27.4, 24.0, 23.1, 15.7

IR (film, cm⁻¹) 3227 (br), 2976, 2868, 1599, 1339, 1256, 1167, 1098, 737

HRMS (ESI) m/z calcd for C₂₉H₄₂N₃O₄ (M+H)⁺ 496.3175. found 496.3171 (0.9 ppm).

### 2at′-Me. (S)-5-((R)-1-(Benzyloxy)ethyl)-4-methoxy-1-((S)-1-((S)-2-methyl-5-oxo-2,5-dihydro-1H-pyrrol-3-yl)pyrrolidin-3-yl)-1H-pyrrol-2(5H)-one

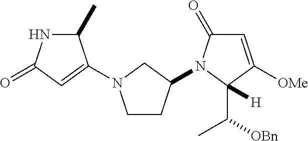 [SMILES: COC1=CC(=O)N([C@H]2CCN(C3=CC(=O)N[C@H]3C)C2)[C@H]1[C@@H](C)OCc1ccccc1]

Procedure: As per the general procedure for the one-pot coupling reaction.

Physical state: White solid (51%).

¹H-NMR (300 MHz, CDCl₃) δ 7.35-7.24 (m, 5H), 5.68 (br s, 1H), 5.04 (s, 1H), 4.70 (d, J=12.0 Hz, 1H), 4.50-4.41 (m, 2H), 4.11-3.96 (m, 3H), 3.78 (s, 3H), 3.78-3.70 (m, 1H), 3.67-3.55 (m, 1H), 3.33-3.21 (m, 2H), 3.21-3.08 (m, 1H), 2.42-2.25 (m, 1H), 2.01-1.88 (m, 1H), 1.30 (d, J=6.6 Hz, 3H), 1.18 (d, J=6.0 Hz, 3H)

¹³C-NMR (75 MHz, CDCl₃) δ 176.1, 175.0, 172.7, 167.2, 137.7, 128.6, 128.0, 127.7, 95.8, 87.7, 74.0, 71.1, 64.5, 58.3, 53.6, 52.8, 49.7, 47.3, 27.2, 19.0, 14.8

IR (film, cm⁻¹) 3260 (br), 2928, 2868, 1670, 1595, 1396, 1361, 1238, 1099, 995, 733, 696

HRMS (ESI) m/z calcd for C₂₃H₃₀N₃O₄ (M+H)⁺ 412.2236. found 412.2277 (3.7 ppm).

### 2ma-^{t}Bu. (S)-4-(tert-butoxy)-5-methyl-1-((S)-1-((S)-2-(2-(methylthio)ethyl)-5-oxo-2,5-dihydro-1H-pyrrol-3-yl)pyrrolidin-3-yl)-1H-pyrrol-2(5H)-one

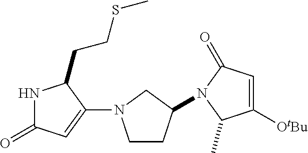 [SMILES: CSCC[C@@H]1NC(=O)C=C1N1CC[C@H](N2C(=O)C=C(OC(C)(C)C)[C@@H]2C)C1]

Procedure: As described in the literature from the corresponding tetramic acid 2 and amine 5; 59%.¹⁵

Physical state: pale yellow oil

¹H-NMR (300 MHz, CDCl₃) δ 6.37 (br s, 1H), 5.00 (app s, 1H), 4.56 (d, J=1.2 Hz, 1H), 4.37-4.25 (m, 2H), 3.94-3.82 (m, 1H), 3.64-3.52 (m, 1H), 3.53-3.38 (m, 2H), 3.38-3.30 (m, 1H), 2.57 (t, J=7.3 Hz, 2H), 2.53-2.40 (m, 1H), 2.28-2.13 (m, 2H), 2.11 (s, 3H), 1.90-1.75 (m, 1H), 1.45 (s, 9H), 1.35 (d, J=6.6 Hz, 3H)

¹³C-NMR (75 MHz, CDCl₃) δ 176.2, 172.7, 172.1, 165.4, 95.5, 89.2, 81.9, 77.3, 57.9, 56.3, 51.7, 50.1, 47.6, 31.9, 29.9, 27.4, 18.0, 15.7

IR (film, cm⁻¹) 3214 (br), 2958, 2873, 1653, 1599, 1480, 1456, 1399, 1373, 1339, 1302, 1259, 1214, 1168, 1096, 880, 840, 781, 757

HRMS (ESI) m/z calcd for (M+H)⁺ C₂₀H₃₂N₃O₃S 394.2164. found 394.2175 (2.7 ppm).

### 2mi-^{t}Bu. (S)-4-(tert-butoxy)-5-((S)-sec-butyl)-1-((S)-1-((S)-2-isobutyl-5-oxo-2,5-dihydro-1H-pyrrol-3-yl)pyrrolidin-3-yl)-1H-pyrrol-2(5H)-one

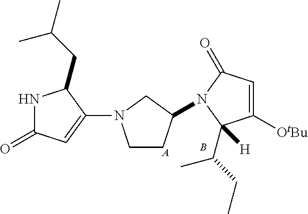

Procedure: As described in the literature from the corresponding tetramic acid 2 and amine 5, 55%.¹⁵

Physical state: Pale yellow solid

¹H-NMR (300 MHz, CDCl₃) δ 5.70 (br s, 1H), 4.99 (s, 1H), 4.52 (d, J=1.5 Hz, 1H), 4.14 (d, J=9.6 Hz, 1H), 4.07-3.88 (m, 1H), 3.86 (d, J=2.7 Hz, 1H), 3.83-3.69 (m, 1H), 3.46-3.15 (m, 3H), 2.74-2.52 (m, 1H), 1.87-1.47 (m, 5H), 1.42 (s, 9H), 1.40-1.35 (m, 1H), 1.0-0.89 (m, 6H), 0.75 (d, J=6.9 Hz, 3H)

¹³C-NMR (75 MHz, CDCl₃) δ 176.2, 173.3, 170.4, 166.7, 97.4, 88.3, 82.0, 65.8, 55.7, 52.6, 49.6, 47.4, 42.2, 36.4, 27.4, 26.0, 25.9, 23.7, 21.4, 12.6, 12.4

Note: Carbons A and B overlap at 36.4

HRMS (ESI) m/z calcd for C₂₄H₄₀N₃O₃ (M+H)⁺ 418.3070. found 418.3083 (3.1 ppm).

### 2le′-^{t}Bu. tert-Butyl 3-((S)-3-(tert-butoxy)-1-((S)-1-((S)-2-isobutyl-5-oxo-2,5-dihydro-1H-pyrrol-3-yl)pyrrolidin-3-yl)-5-oxo-2,5-dihydro-1H-pyrrol-2-yl)propanoate

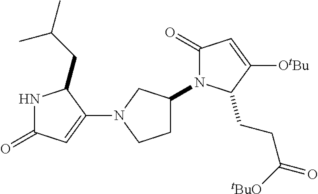 [SMILES: CC(C)C[C@@H]1NC(=O)C=C1N1CC[C@H](N2C(=O)C=C(OC(C)(C)C)[C@@H]2CCC(=O)OC(C)(C)C)C1]

Procedure: As per the general procedure for the one-pot coupling reaction.

Physical state: White solid (49%).

¹H-NMR (300 MHz, CDCl₃) δ 5.71 (br s, 1H), 5.02 (s, 1H), 4.51 (s, 1H), 4.29-4.10 (m, 2H), 4.01-3.94 (m, 1H), 3.67-3.54 (m, 1H), 3.48-3.32 (m, 2H), 3.32-3.19 (m, 1H), 2.57-2.37 (m, 2H), 2.26-1.96 (m, 5H), 1.80-1.67 (m, 1H), 1.66-1.53 (m, 1H), 1.43 (s, 9H), 1.41 (s, 9H), 0.95 (d, J=6.6 Hz, 3H), 0.92 (d, J=6.6 Hz, 3H)

¹³C-NMR (75 MHz, CDCl₃) δ 176.2, 173.2, 172.2, 170.0, 166.7, 97.0, 88.4, 82.3, 80.8, 60.7, 55.7, 52.0, 49.7, 47.4, 42.1, 28.1, 27.4, 25.8, 24.7, 23.7, 21.4

IR (film, cm⁻¹) 3123 (br), 2976, 2871, 1724, 1670, 1600, 1395, 1369, 1341, 1258, 1167, 922, 887, 847, 731

HRMS (ESI) m/z calcd for C₂₇H₄₄N₃O₅ (M+H)⁺ 490.3281. found 490.3270 (2.2 ppm).

### 2t′a-^{t}Bu. (S)-1-((S)-1-((R)-2-((R)-1-(Benzyloxy)ethyl)-5-oxo-2,5-dihydro-1H-pyrrol-3-yl)pyrrolidin-3-yl)-4-(tert-butoxy)-5-methyl-1H-pyrrol-2(5H)-one

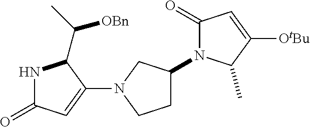 [SMILES: C[C@@H](OCc1ccccc1)[C@@H]1NC(=O)C=C1N1CC[C@H](N2C(=O)C=C(OC(C)(C)C)[C@@H]2C)C1]

Procedure: As described in the literature from the corresponding tetramic acid 2 and amine 5.¹⁵

Physical state: White solid (67%).

¹H-NMR (300 MHz, CDCl₃) δ 7.37-7.19 (m, 5H), 6.29 (br s, 1H), 4.94 (s, 1H), 4.62-4.54 (m, 2H), 4.43 (d, J=11.7 Hz, 1H), 4.39-4.26 (m, 1H), 4.24 (d, J=1.5 Hz, 1H), 3.87-3.76 (m, 1H), 3.69-3.58 (m, 1H), 3.38-3.24 (m, 3H), 3.23-3.09 (m, 1H), 2.30-2.04 (m, 2H), 1.42 (s, 9H), 1.23 (d, J=6.6 Hz, 3H), 1.17 (d, J=6.3 Hz, 3H)

¹³C-NMR (75 MHz, CDCl₃) δ 176.7, 172.7, 172.0, 163.8, 138.1, 128.3, 127.8, 127.7, 95.2, 90.6, 81.8, 74.2, 71.1, 61.2, 57.3, 51.0, 50.9, 48.1, 29.0, 27.4, 18.2, 15.3

IR (film, cm⁻¹) 3250 (br), 2978, 2872, 1597, 1398, 1375, 1257, 1213, 1167, 1096, 735

MS (ESI) m/z calcd for C₂₆H₃₆N₃O₄ (M+H)⁺ 454.27. found 454.26.

### 2fe′-^{t}Bu. Tert-butyl 3-((S)-1-((S)-1-((S)-2-benzyl-5-oxo-2,5-dihydro-1H-pyrrol-3-yl)pyrrolidin-3-yl)-3-(tert-butoxy)-5-oxo-2,5-dihydro-1H-pyrrol-2-yl)propanoate

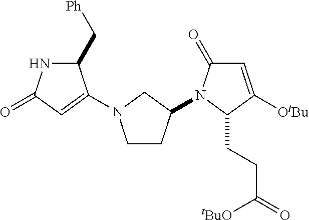 [SMILES: CC(C)(C)OC(=O)CC[C@H]1C(OC(C)(C)C)=CC(=O)N1[C@H]1CCN(C2=CC(=O)N[C@H]2Cc2ccccc2)C1]

Procedure: As described in the literature from the corresponding tetramic acid 2 and amine 5; 64%.¹⁵

Physical state: Colorless oil that crashed into a solid under vacuum (64%).

¹H-NMR (300 MHz, CDCl₃) δ 7.35-7.13 (m, 5H), 5.23 (br s, 1H), 5.03 (s, 1H), 4.52 (s, 1H), 4.32 (dd, J=9.9, 3.0 Hz, 1H), 0.3.04-4.14 (m, 1H), 4.04-3.95 (m, 1H), 3.82-3.66 (m, 1H), 3.65-3.53 (m, 1H), 3.52-3.30 (m, 2H), 3.25 (dd, J=13.5, 3.0 Hz, 1H), 2.66-2.41 (m, 2H), 2.30-2.16 (m, 1H), 2.15-1.95 (m, 4H), 1.44 (s, 9H), 1.43 (s, 9H)

¹³C-NMR (75 MHz, CDCl₃) δ 175.6, 173.3, 172.3, 170.1, 165.5, 136.8, 129.1, 128.8, 128.6, 127.1, 97.0, 88.8, 82.3, 80.9, 60.7, 58.4, 52.1, 49.9, 47.7, 39.4, 28.1, 27.4, 24.8

IR (film, cm⁻¹) 3250 (br), 2978, 2931, 2872, 1724, 1667, 1601, 1395, 1371, 1339, 1371, 1339, 1258, 1165, 1151, 702

HRMS (ESI) m/z calcd for C₃₀H₄₂N₃O₅ (M+H)⁺ 524.3124. found 524.3115 (1.8 ppm).

### 2wl-^{t}Bu. (S)-1-((S)-1-((S)-2-((1H-Indol-3-yl)methyl)-5-oxo-2,5-dihydro-1H-pyrrol-3-yl)pyrrolidin-3-yl)-4-(tert-butoxy)-5-isobutyl-1H-pyrrol-2(5H)-one

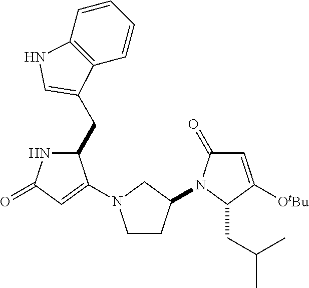 [SMILES: CC(C)C[C@H]1C(OC(C)(C)C)=CC(=O)N1[C@H]1CCN(C2=CC(=O)N[C@H]2Cc2c[nH]c3ccccc23)C1]

Procedure: As described in the literature from the corresponding tetramic acid 2 and amine 5; 52%.¹⁵

Physical state: Pale yellow solid

¹H-NMR (300 MHz, CDCl₃) δ 9.79 (s, 1H), 7.47 (d, J=7.5 Hz, 1H), 7.36 (d, J=7.8 Hz, 1H), 7.14 (t, J=7.8 Hz, 1H), 7.06 (t, J=7.2 Hz, 1H), 6.95 (d, J=1.5 Hz, 1H), 5.35 (br s, 1H), 5.00 (s, 1H), 4.56 (s, 1H), 4.38-4.28 (m, 1H), 4.26-4.12 (m, 1H), 3.94-3.76 (m, 2H), 3.76-3.55 (m, 2H), 3.49-3.31 (m, 2H), 2.69 (dd, J=14.7, 9.6 Hz, 1H), 2.59-2.41 (m, 1H), 2.28-2.13 (m, 1H), 1.91-1.74 (m, 1H), 1.69-1.56 (m, 2H), 1.44 (s, 9H), 0.94 (d, J=0.9 Hz, 3H), 0.92 (d, J=0.9 Hz, 3H)

¹³C-NMR (75 MHz, CDCl₃) δ 176.4, 173.4, 171.9, 166.2, 136.6, 126.9, 123.4, 121.9, 119.3, 118.0, 111.9, 110.4, 96.2, 88.9, 82.1, 61.4, 57.5, 52.4, 50.9, 47.8, 39.6, 30.2, 28.8, 27.4, 24.1, 24.0, 23.1

IR (film, cm⁻¹) 3414, 3246 (br), 2976, 2928, 2868, 1647, 1597, 1422, 1341, 1167, 908, 735

HRMS (ESI) m/z calcd for C₂₉H₃₉N₄O₃ (M+H)⁺ 491.3022. found 491.3035 (2.6 ppm).

### Compound 1lai-^{t}Bu: (S)-4-(tert-butoxy)-5-((S)-sec-butyl)-1-((S)-1-((S)-1-((S)-1-((S)-2-isobutyl-5-oxo-2,5-dihydro-1H-pyrrol-3-yl)pyrrolidin-3-yl)-2-methyl-5-oxo-2,5-dihydro-1H-pyrrol-3-yl)pyrrolidin-3-yl)-1H-pyrrol-2(5H)-one

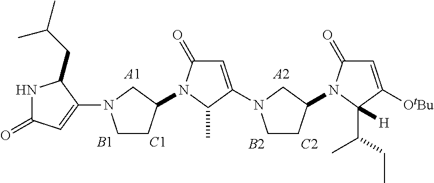

Procedure: Synthesized from 2la and 6i using the procedure reported above for 1aaa-^{t}Bu. 55% yield; ¹H-NMR (300 MHz, CDCl₃) δ 5.72 (br s, 1H), 4.97 (s, 1H), 4.51 (s, 1H), 4.49 (s, 1H), 4.29-3.90 (m, 4H), 3.84 (d, J=2.4 Hz, 1H), 3.82-3.71 (m, 1H), 3.62-3.46 (m, 1H), 3.46-3.32 (m, 4H), 3.30-3.15 (m, 2H), 2.73-2.52 (m, 1H), 2.50-2.29 (m, 2H), 2.26-2.02 (m, 2H), 1.84-1.44 (m, 5H), 1.40 (s, 9H), 1.42-1.34 (m, 3H), 1.02-0.87 (m, 9H), 0.74 (d, J=6.9 Hz, 3H); HRMS (ESI) m/z calcd for (M+H)⁺ C₃₀H₃₆N₃O₃ 582.4019. found 582.4029 (1.5 ppm).

### 1lai-H. (3′S,5S)-5-((S)-sec-butyl)-1′-((S)-1-((S)-1-((S)-2-Isobutyl-5-oxo-2,5-dihydro-1H-pyrrol-3-yl)pyrrolidin-3-yl)-2-methyl-5-oxo-2,5-dihydro-1H-pyrrol-3-yl)-[1,3′-bipyrrolidine]-2,4-dione

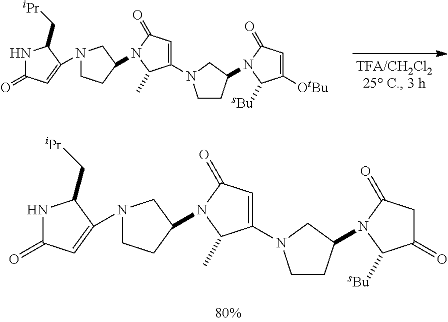

Procedure: As described in the literature.

Physical state: Off-white solid, 80%.

¹H-NMR (300 MHz, CDCl₃) δ 6.01 (br s, 1H), 4.65-4.48 (m, 2H), 4.27-4.02 (m, 4H), 3.93 (d, J=3.0 Hz, 1H), 3.77 (t, J=9.5 Hz, 1H), 3.68-3.51 (m, 3H), 3.50-3.37 (m, 3H), 3.37-3.22 (m, 1H), 2.98 (s, 2H), 2.69-2.42 (m, 2H), 2.36-2.11 (m, 2H), 1.93-1.78 (m, 1H), 1.77-1.48 (m, 2H), 1.47-1.32 (m, 3H), 1.03-0.92 (m, 12H), 0.89 (d, J=6.6 Hz, 3H)

¹³C-NMR (75 MHz, CDCl₃) δ 205.1, 176.2, 173.2, 169.7, 167.1, 165.0, 89.7, 87.8, 71.4, 56.2, 55.9, 53.2, 52.2, 51.8, 50.1, 49.7, 47.4, 47.3, 43.3, 41.9, 37.5, 27.8, 25.8, 25.3, 23.7, 21.4, 18.6, 13.3, 12.1

HRMS (MALDI) m/z calcd for C₂₉H₄₄N₅O₄ (M+H)⁺ 526.3393. found 526.3381 (2.3 ppm).

### 1fla-^{t}Bu. (S)-1-((S)-1-((S)-2-benzyl-5-oxo-2,5-dihydro-1H-pyrrol-3-yl)pyrrolidin-3-yl)-4-((S)-3-((S)-3-(tert-butoxy)-2-methyl-5-oxo-2,5-dihydro-1H-pyrrol-1-yl)pyrrolidin-1-yl)-5-isobutyl-1H-pyrrol-2(5H)-one

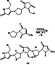

Procedure: As described in the literature.¹⁵

Physical state: White solid (54%)

¹H-NMR (300 MHz, CDCl₃) δ 7.32-7.14 (m, 5H), 5.11 (br s, 1H), 4.96 (s, 1H), 4.58 (s, 1H), 4.50 (s, 1H), 4.33-4.23 (m, 2H), 4.24-4.08 (m, 2H), 3.86 (q, J=6.6 Hz, 1H), 3.63.320 (m, 8H), 2.66-2.38 (m, 4H), 2.26-2.08 (m, 2H), 1.77-1.59 (m, 3H), 1.42 (s, 9H), 1.32 (d, J=6.6 Hz, 3H), 0.91 (d, J=6.3 Hz, 3H), 0.86 (d, J=6.0 Hz, 3H)

¹³C-NMR (75 MHz, CDCl₃) δ 175.5, 174.2, 172.7, 172.1, 165.5, 163.5, 137.0, 129.1, 128.8, 127.1, 95.5, 90.6, 88.6, 82.0, 60.2, 58.5, 57.8, 52.9, 51.7, 50.3, 49.9, 47.6, 47.5, 39.4, 38.2, 27.4, 24.1, 23.9, 23.1, 18.0

Note: Carbons A1, A2 and B1, B2 overlap

HRMS (MALDI) m/z calcd for C₃₆H₅₀N₅O₄ (M+h)⁺ 616.3857. found 616.3865 (1.3 ppm).

### 2afe′-^{t}Bu. Tert-butyl 3-((S)-1-((S)-1-((S)-2-benzyl-1-((S)-1-((S)-2-methyl-5-oxo-2,5-dihydro-1H-pyrrol-3-yl)pyrrolidin-3-yl)-5-oxo-2,5-dihydro-1H-pyrrol-3-yl)pyrrolidin-3-yl)-3-(tert-butoxy)-5-oxo-2,5-dihydro-1H-pyrrol-2-yl)propanoate

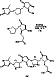

Procedure: As described in the literature.¹⁵

Physical state: Pale yellow solid, 42%.

¹H-NMR (300 MHz, CDCl₃) δ 7.25-7.01 (m, 5H), 5.72 (br s, 1H), 5.00 (s, 1H), 4.41 (s, 1H), 4.38 (s, 1H), 4.32 (t, J=4.5 Hz, 1H), 4.25-4.16 (s, 1H), 4.15-4.05 (s, 1H), 4.01-3.82 (m, 2H), 3.71-3.42 (m, 3H), 3.42-3.17 (m, 4H), 3.17-3.05 (m, 2H), 2.91 (dd, J=14.3, 5.3 Hz, 1H), 2.55-2.29 (m, 2H), 2.25-2.13 (m, 1H), 2.12-1.95 (m, 5H), 1.42 (s, 9H), 1.40 (s, 9H), 1.31 (d, J=6.3 Hz, 3H)

¹³C-NMR (75 MHz, CDCl₃) δ 176.2, 174.3, 173.3, 172.3, 170.1, 167.3, 163.5, 135.1, 129.3, 128.3, 127.1, 96.9, 91.1, 87.4, 82.4, 80.8, 61.2, 60.6, 53.4, 52.9, 52.0, 50.1, 50.0, 47.6, 47.4, 37.6, 28.1, 27.4, 24.7, 19.0

Note: Carbons A1 and A1, B1 and B2, C1 and C2 overlap.

IR (film, cm⁻¹) 3123 (br), 2976, 2871, 1724, 1670, 1600, 1395, 1369, 1341, 1258, 1167, 922, 887, 847, 731

HRMS (MALDI) m/z calcd for C₃₉H₅₄N₅O₆ (M+H)⁺ 688.4067. found 688.4045 (3.2 ppm).

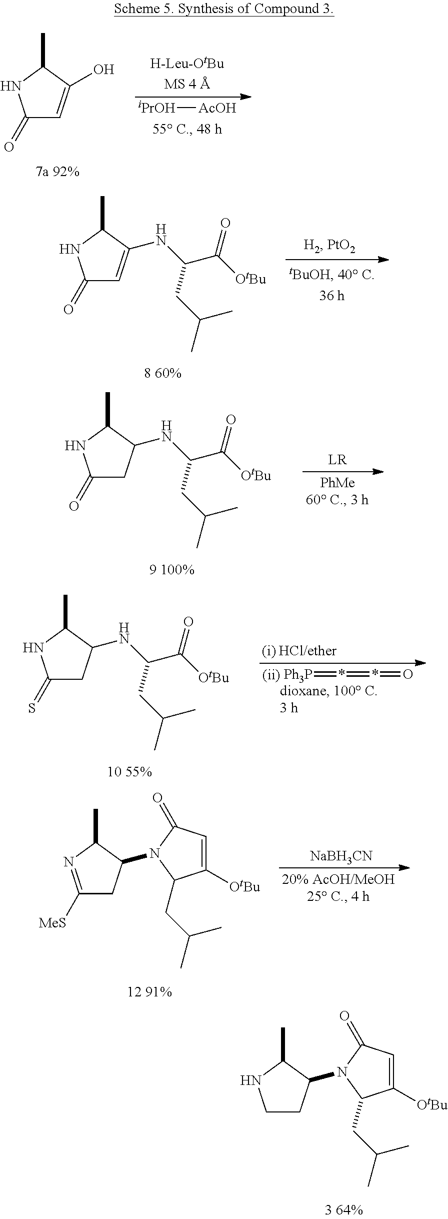

### Compound 8: ((S)-tert-butyl 4-methyl-2-((S)-2-methyl-5-oxo-2,5-dihydro-1H-pyrrol-3-yl)amino)pentanoate

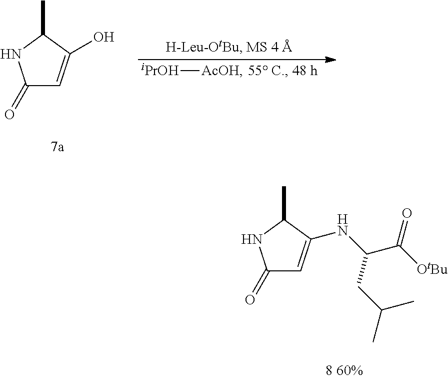

Procedure: A modified procedure was used.⁴ The tetramic acid 7a (1.85 g, 16.4 mmol, 1.5 equiv) and leucine tert-butyl ester (2.51 g, 11 mmol, 1 equiv) was stirred in 9:1 ⁱPrOH/AcOH (55 mL, 0.2 M with respect to leucine tert-butyl ester) in the presence of 4 Å molecular sieves. The reaction was purged with N₂ and heated to 55° C. for 48 h. Upon cooling, the reaction mixture was filtered over Celite. Toluene (50 mL) was added and the solution was concentrated. Residual AcOH was azeotroped with PhMe (3×50 mL) to obtain a brown residue. Purification by flash chromatography (1-2% MeOH/EtOAc) afforded the product as a brown solid, which was further purified by crystallization from hot EtOAc to obtain 8 in 60% yield. [α]²⁰−93.1 (c 1.0, MeOH); ¹H-NMR (300 MHz, CDCl₃) δ 6.40 (br s, 1H), 5.36 (d, J=8.1 Hz, 1H), 4.60 (s, 1H), 4.09 (m, 1H), 3.76 (q, J=7.5 Hz, 1H), 1.72 (m, 1H), 1.61 (t, J=6.7 Hz, 2H), 1.45 (s, 9H), 1.35 (d, J=6.9 Hz, 3H), 0.94 (d, J=6.6 Hz, 3H), 0.91 (d, J=6.6 Hz, 3H); MS (ESI) m/z calcd for (M+H)⁺ C₁₅H₂₇N₂O₃ 283.19. found 283.19.

### Compound 9: (S)-tert-butyl 4-methyl-2-(((2S,3S)-2-methyl-5-oxopyrrolidin-3-yl)amino)pentanoate

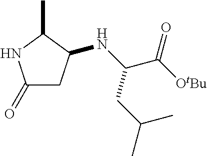 [SMILES: CC(C)C[C@H](N[C@H]1CC(=O)N[C@H]1C)C(=O)OC(C)(C)C]

Procedure: As described in the literature. [α]²⁰−10.0 (c 1.0, MeOH); ¹H-NMR (300 MHz, CDCl₃) δ 7.20 (br s, 1H), 3.70 (m, 1H), 3.35 (m, 1H), 3.01 (app t, J=7.3 Hz, 1H), 2.32 (dd, J=16.4, 7.7 Hz, 1H), 2.14 (dd, J=16.4, 9.6 Hz, 1H), 1.72 (m, 1H), 1.41 (s, 9H), 1.34 (m, 2H), 1.08 (d, J=6.3 Hz, 3H), 0.88 (d, J=6.3 Hz, 3H), 0.85 (d, J=6.6 Hz, 3H); MS (ESI) m/z calcd for (M+H)⁺ C₁₅H₂₉N₂O₃ 285.21. found 285.19.

### Compound 10: (S)-tert-butyl 4-methyl-2-(((2S,3S)-2-methyl-5-thioxopyrrolidin-3-yl)amino)pentanoate

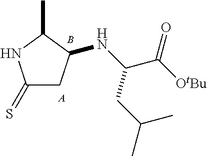

Procedure: The amine 9 (440 mg, 1.55 mmol, 1 equiv) was dissolved in dry toluene (16 mL) and the reaction flask was evacuated and re-filled with N₂. Lawesson's reagent (314 mg, 0.78 mmol, 0.5 equiv) was added under a stream of N₂ and the reaction was heated to 60° C. Upon heating the reaction became clear. After 3 h, the reaction mixture was concentrated in a fume hood. Ether (40 mL) was added to the residue and stirred vigorously for 4 h. The ether layer was decanted and concentrated to obtain the crude product. The pure product was obtained by flash chromatography (SiO₂, PhMe then 20% EtOAc/Hexanes) in 55% yield; [α]²°+13.1 (c 0.5, MeOH); ¹H-NMR (300 MHz, CDCl₃) δ 8.4 (br s, 1H), 3.98 (m, 1H), 3.48 (app q, J=7.5 Hz, 1H), 3.08-2.98 (m, 2H), 2.72 (dd, J=17.9, 8.5 Hz, 1H), 1.79 (m, 1H), 1.46 (s, 9H), 1.40 (m, 2H), 1.23 (d, J=6.6 Hz, 3H), 0.94 (d, J=6.9 Hz, 3H), 0.91 (d, J=6.9 Hz, 3H); MS (ESI) m/z calcd for (M+H)⁺ C₁₅H₂₉N₂O₂S 301.19. found 301.17.

### Compound 11: (S)-4-(tert-butoxy)-5-isobutyl-1-((2S,3S)-2-methyl-5-thioxopyrrolidin-3-yl)-1H-pyrrol-2(5H)-one

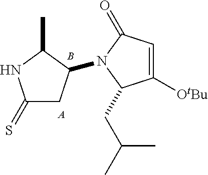

Procedure: The amine 10 (200 mg, 0.67 mmol) was dissolved in dry ether (15 mL) and cooled to 0° C. HCl/ether (2M, 0.37 mL) was added dropwise to precipitate the hydrochloride salt. After 10 min, the solution was filtered using a sintered glass funnel and washed with cold Et₂O (10 mL). Since the hydrochloride salt was hygroscopic, MeOH (10 mL) was added to the funnel to dissolve the salt. The MeOH filtrate was concentrated to obtain 10.HCl.

To a solution of 10.HCl (206 mg, 0.61 mmol) in dioxane (7 mL) at 100° C. under an Argon atmosphere was added Bestmann's ylide (2× re-crystallized from PhMe, 221 mg, 0.73 mmol, 1.2 equiv) in one portion. After 30 min, a second portion of Bestmann's ylide (37 mg, 0.12 mmol, 0.2 equiv) was added, and this process was repeated three additional times at 15 min intervals to complete the addition of 2 equiv of ylide. The reaction was monitored by NMR spectroscopy.

After completion of reaction (˜3 h), the solvent was removed. The product was isolated by flash chromatography (5-10% acetone/dichlormethane). Further purification was achieved by crystallization to obtain 130 mg of 11 60% yield. [α]²⁰−58.5 (c 1.0, MeOH); ¹H-NMR (300 MHz, CDCl₃) δ 9.0 (s, 1H), 5.09 (s, 1H), 4.82-4.66 (m, 1H), 4.37-4.24 (m, 1H), 4.01-3.94 (m, 1H), 3.31 (dd, J=17.4, 6.0 Hz, 1H), 3.12 (dd, J=17.6, 8.3 Hz, 1H), 1.81-1.64 (m, 3H), 1.45 (s, 9H), 1.12 (d, J=6.6 Hz, 3H), 0.98-0.85 (m, 6H); MS (ESI) m/z calcd for (M+H)⁺ C₁₇H₂₉N₂O₂S 325.19. found 325.20.

### Compound 12: (2′S,3′S,5S)-4-(tert-butoxy)-5-isobutyl-2′-methyl-5′-(methylthio)-3′,4′-dihydro-2′H-[1,3′-bipyrrol]-2(5H)-one

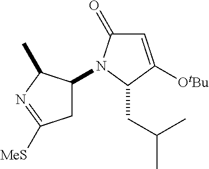

Procedure: To a solution of 11 (98 mg, 0.3 mmol, 1 equiv) in THF (3 mL) was added potassium bicarbonate (45 mg, 0.45 mmol, 1.5 equiv) followed by methyl iodide (28 μL, 0.45 mmol, 1.5 equiv) dropwise. The reaction was stirred at 25° C. for 12 h during the course of which a white precipitate formed. The precipitate was filtered and the filtrate was evaporated to obtain the pure product in 91% yield; ¹H-NMR (300 MHz, CDCl₃) δ 5.01 (s, 1H), 4.79-4.69 (m, 1H), 4.18 (p, J=6.6 Hz, 1H), 3.81-3.72 (m, 1H), 3.06 (dd, J=16.8, 3.9 Hz, 1H), 2.84 (dd, J=16.8, 8.7 Hz, 1H), 2.50 (s, 3H), 1.89-1.78 (m, 1H), 1.64-1.58 (m, 2H), 1.43 (s, 9H), 1.15 (d, J=7.2 Hz, 3H), 0.92 (d, J=6.6 Hz, 3H), 0.89 (d, J=6.6 Hz, 3H); HRMS (ESI) m/z calcd for (M+H)⁺ 339.2106. found 339.2102 (1.3 ppm).

### Compound 3: (S)-4-(tert-butoxy)-5-isobutyl-1-((2S,3S)-2-methylpyrrolidin-3-yl)-1H-pyrrol-2(5H)-one

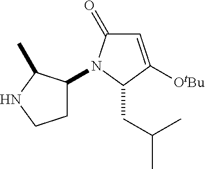

Procedure: To a solution of 12 (43 mg, 0.13 mmol, 1 equiv) in 20% AcOH/MeOH (1.3 mL) at 25° C. was added sodium cyanoborohydride (32 mg, 0.5 mmol, 4 equiv in one portion. This process was repeated 3 additional times every 1 hour. After 4 h, the reaction was brought 0° C. and carefully neutralized with 2 N NaOH (2.5 mL). Ether (10 mL) was added and extracted with saturated NaHCO₃ (5 mL) and brine (5 mL). The organic layer was dried over MgSO₄, filtered and concentrated in a fume hood. The residue was purified by flash chromatography to afford 23 mg of the product as a white solid; ¹H-NMR (300 MHz, CDCl₃) δ 9.6 (br, 1H), 8.8 (br, 1H), 5.09 (s, 1H), 4.01 (t, J=7.2 Hz, 1H), 3.89 (dd, J=7.5, 4.8 Hz, 1H), 3.84-3.70 (m, 1H), 3.62-3.50 (m, 1H), 3.51-3.28 (m, 1H), 2.78-2.64 (m, 1H), 2.11-1.90 (m, 2H), 1.61-1.54 (m, 2H), 1.50 (s, 9H), 1.44 (d, J=6.6 Hz, 3H), 0.99 (app d, 6H); MS (ESI) m/z calcd for (M+H)⁺ C₁₇H₃₁N₂O₂ 295.23. found 295.24.

### Part II. Pyrrolinone-Piperidine Oligomers

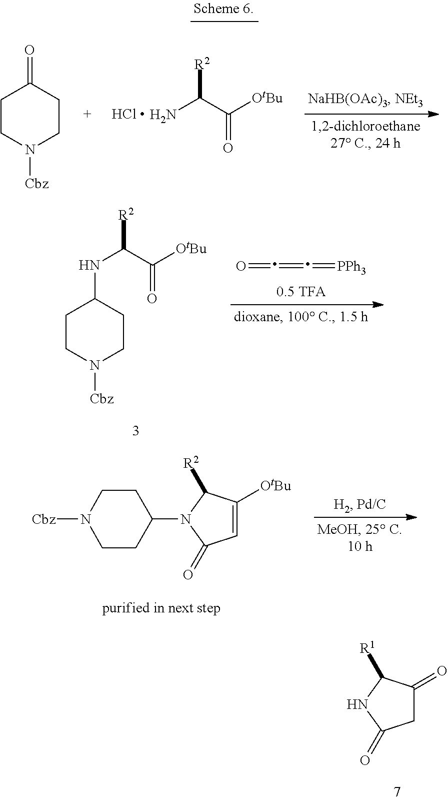

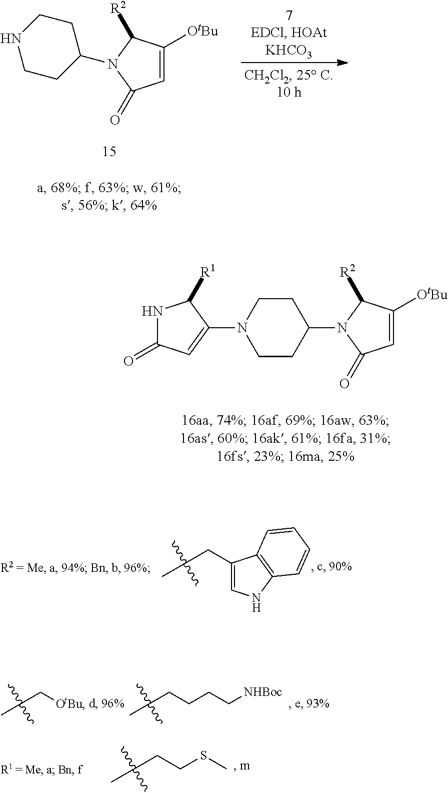

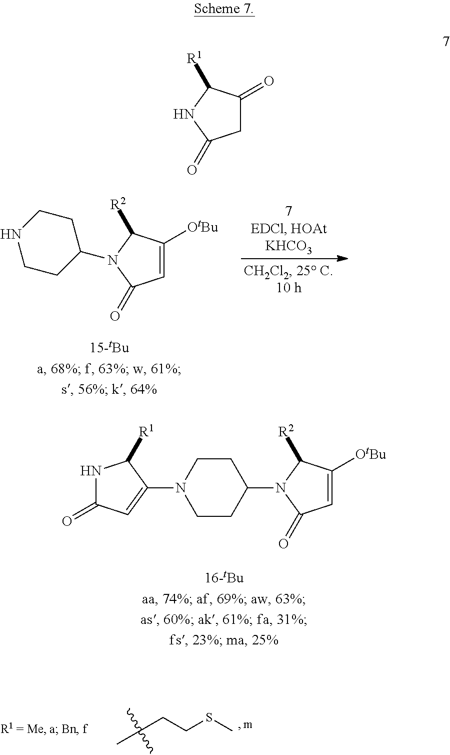

### Compound 14aaa

### (S)-4-Methoxy-6-methyl-1-(1-((S)-2-methyl-1-(1-((S)-2-methyl-6-oxo-1,2,3,6-tetrahydropyridin-4-yl)piperidin-4-yl)-6-oxo-1,2,3,6-tetrahydropyridin-4-yl)piperidin-4-yl)-5,6-dihydropyridin-2(1H)-one

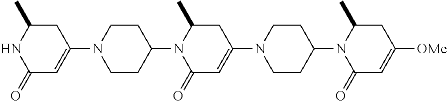 [SMILES: COC1=CC(=O)N(C2CCN(C3=CC(=O)N(C4CCN(C5=CC(=O)N[C@@H](C)C5)CC4)[C@@H](C)C3)CC2)[C@@H](C)C1]

Light yellow oil, 85%;

¹H-NMR (300 MHz, CDCl₃) δ 5.33 (s, 1H), 5.12 (d, J=1.8 Hz, 1H), 4.97 (s, 1H), 4.90 (s, 1H), 4.76-4.57 (m, 2H), 3.84-3.61 (m, 10H), 3.01-2.69 (m, 6H), 2.58-2.50 (m, 1H), 2.49-2.39 (m, 1H), 2.12-1.98 (m, 2H), 1.90-1.57 (m, 8H), 1.34-1.27 (m, 9H);

¹³C-NMR (75 MHz, CDCl₃) δ 170.9, 166.5, 165.9, 165.4, 157.5, 153.8, 94.0, 92.8, 90.5, 55.6, 50.5, 49.9, 46.3, 46.2, 46.1, 45.9, 45.4, 45.2, 45.1, 35.3, 34.0, 33.4, 30.2, 29.9, 29.1, 28.9, 21.2, 20.4, 20.3;

IR (film, cm⁻¹) 3406, 2927, 2361, 1603, 1433, 1377, 1321, 1276, 1207, 1175, 1005, 808, 731;

HRMS (ESI) m/z calcd for C₂₉H₄₃N₅O₄ (M+H)⁺ 526.3393. found 526.3411 (3.4 ppm).

### Compound 14lat′

### (R)-6-((R)-1-(Benzyloxy)ethyl)-1-(1-((S)-1-(1-((R)-2-isobutyl-6-oxo-1,2,3,6-tetrahydropyridin-4-yl)piperidin-4-yl)-2-methyl-6-oxo-1,2,3,6-tetrahydropyridin-4-yl)piperidin-4-yl)-4-methoxy-5,6-dihydropyridin-2(1H)-one

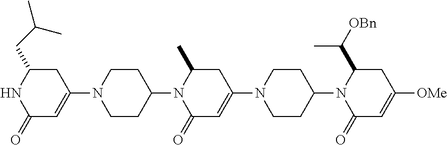 [SMILES: COC1=CC(=O)N(C2CCN(C3=CC(=O)N(C4CCN(C5=CC(=O)N[C@H](CC(C)C)C5)CC4)[C@@H](C)C3)CC2)[C@@H](C(C)OCc2ccccc2)C1]

Colorless oil, 77%;

¹H-NMR (300 MHz, CDCl₃) δ 7.29-7.20 (m, 5H), 5.10 (s, 1H), 4.97 (s, 1H), 4.83 (s, 2H), 4.65-4.47 (m, 2H), 4.32 (d, J=11.7 Hz, 1H), 4.18-4.03 (m, 1H), 3.76-3.38 (m, 11H), 2.94-2.75 (m, 2H), 2.70-2.24 (m, 6H), 2.18-2.01 (m, 2H), 1.92-1.41 (m, 10H), 1.37-1.21 (m, 1H), 1.29 (d, J=6.3 Hz, 3H), 1.07 (d, J=6.3 Hz, 3H), 0.90-0.84 (m, 6H);

¹³C-NMR (75 MHz, CDCl₃) δ 170.7, 166.9, 166.5, 165.9, 157.4, 154.1, 138.1, 128.4, 127.8, 127.7, 94.8, 92.6, 91.0, 75.2, 71.1, 55.7, 55.0, 53.8, 49.9, 48.3, 46.2, 46.0, 45.8, 45.2, 44.4, 33.4, 32.7, 30.0, 29.8, 29.7, 29.4, 29.2, 28.4, 24.3, 23.0, 22.1, 20.3, 15.9;

IR (film, cm⁻¹) 2925, 1614, 1428, 1275, 1224, 1076, 807;

HRMS (ESI) m/z calcd for C₄₀H₅₇N₅O₅ (M+H)⁺ 688.4438. found 688.4463 (3.6 ppm).

### Compound 14vt′a

### (S)-6-((S)-1-(Benzyloxy)ethyl)-1-(1-((R)-2-isopropyl-6-oxo-1,2,3,6-tetrahydropyridin-4-yl)piperidin-4-yl)-4-(4-((R)-4-methoxy-6-methyl-2-oxo-5,6-dihydropyridin-1(2H)-yl)piperidin-1-yl)-5,6-dihydropyridin-2(1H)-one

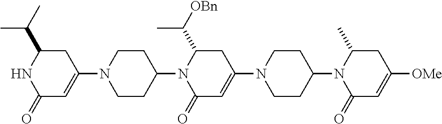 [SMILES: COC1=CC(=O)N(C2CCN(C3=CC(=O)N(C4CCN(C5=CC(=O)N[C@@H](C(C)C)C5)CC4)[C@H](C(C)OCc4ccccc4)C3)CC2)[C@H](C)C1]

Colorless oil, 70%;

¹H-NMR (300 MHz, CDCl₃) δ 7.30-7.20 (m, 5H), 5.29 (br, 1H), 5.04 (s, 1H), 4.83 (s, 1H), 4.79 (s, 1H), 4.59-4.44 (m, 2H), 4.34 (d, J=12.0 Hz, 1H), 4.26-4.10 (m, 1H), 3.73-3.22 (m, 11H), 2.89-2.51 (m, 5H), 2.42-1.84 (m, 5H), 1.82-1.38 (m, 9H), 1.17 (d, J=6.6 Hz, 3H), 1.07 (d, J=6.3 Hz, 3H), 0.95-0.90 (m, 6H);

¹³C-NMR (75 MHz, CDCl₃) δ 171.0, 167.2, 166.5, 165.4, 157.7, 154.4, 138.2, 128.5, 127.8, 127.7, 94.1, 93.7, 90.8, 75.6, 71.2, 56.1, 55.6, 54.5, 52.8, 50.5, 46.2, 46.1, 46.0, 45.9, 45.4, 35.3, 31.8, 30.3, 30.0, 29.7, 29.4, 29.2, 29.1, 20.5, 18.4, 18.3, 15.8;

IR (film, cm⁻¹) 3406 (br), 2927, 2360, 1614, 1433, 1385, 1274, 1243, 1207, 1174, 1078, 1008, 818, 733;

HRMS (ESI) m/z calcd for C₃₉H₅₅N₅O₅ (M+H)⁺ 674.4281. found 674.4301 (3.0 ppm).

### Compound 14lat

### (R)-6-((R)-1-Hydroxyethyl)-1-(1-((S)-1-(1-((R)-2-isobutyl-6-oxo-1,2,3,6-tetrahydropyridin-4-yl)piperidin-4-yl)-2-methyl-6-oxo-1,2,3,6-tetrahydropyridin-4-yl)piperidin-4-yl)-4-methoxy-5,6-dihydropyridin-2(1H)-one

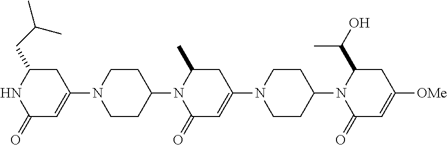 [SMILES: COC1=CC(=O)N(C2CCN(C3=CC(=O)N(C4CCN(C5=CC(=O)N[C@H](CC(C)C)C5)CC4)[C@@H](C)C3)CC2)[C@@H](C(C)O)C1]

White solid, 65%;

¹H-NMR (300 MHz, CDCl₃) δ 5.03 (d, J=1.5 Hz, 1H), 4.90 (s, 1H), 4.87 (s, 1H), 4.66-4.54 (m, 1H), 4.39-4.23 (m, 1H), 3.99-3.85 (m, 1H), 3.82-3.54 (m, 9H), 3.47-3.38 (m, 1H), 2.97-2.43 (m, 6H), 2.42-2.38 (m, 2H), 2.20-2.14 (m, 2H), 1.90-1.44 (m, 11H), 1.38-1.25 (m, 1H), 1.21 (d, J=6.6 Hz, 3H), 1.13 (d, J=6.3 Hz, 3H), 0.96-0.91 (m, 6H);

¹³C-NMR (75 MHz, CDCl₃) δ 170.9, 167.2, 166.7, 166.0, 157.5, 154.1, 94.6, 92.5, 91.0, 67.9, 56.3, 55.7, 53.6, 50.0, 48.3, 46.1, 46.1, 45.9, 45.3, 44.4, 33.4, 32.7, 30.2, 30.1, 29.7, 29.5, 28.9, 28.5, 24.3, 23.0, 22.2, 20.3, 19.4;

IR (film, cm⁻¹) 3286, 2927, 1601, 1432, 1383, 1277, 1225, 1078, 1007, 804;

HRMS (ESI) m/z calcd for C₃₃H₅₁N₅O₅ (M+H)⁺ 598.3968. found 598.3941 (4.3 ppm).

### Part III. Determining Accessible Conformations Illustrated for 1Aaa-H

Provided herein is a method for matching preferred conformations of the small molecules, with orientations of amino acid side-chains at protein-protein interfaces; we call this method Exploring Key Orientations (EKO). EKO could be implemented using different ways of determining preferred conformations of the small molecule, and/or via different versions of a data-mining algorithm, and/or applied to different databases. The common key feature of this method is using a data mining algorithm to compare simulated preferred conformations of a molecule (for example, a semi-rigid organic scaffold) expressing amino acid side chains (preferably 2 or more; more preferably 3) with orientations of side-chains in protein-protein interaction interface regions as determined, for example, by X-ray crystallography or NMR.

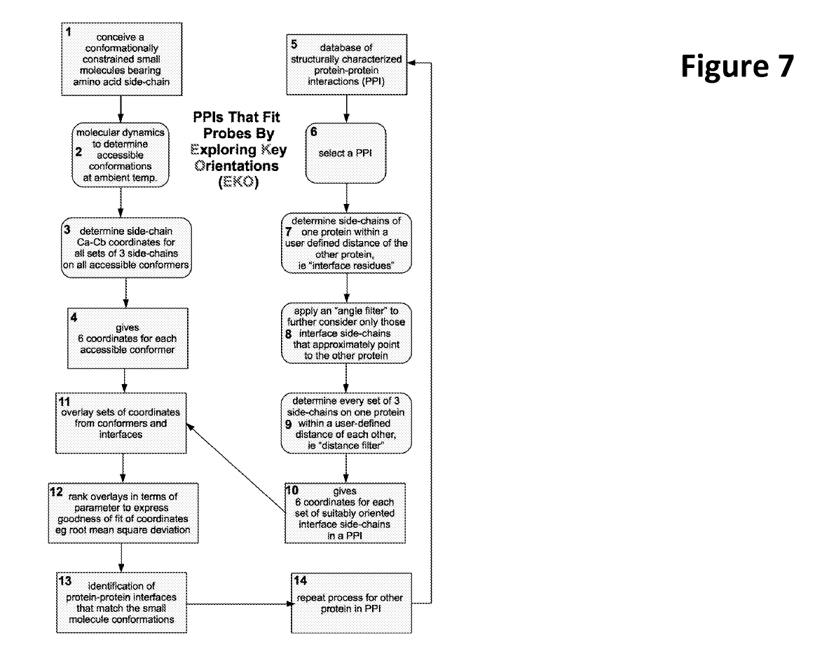

Scaffold 1 is a semi-rigid small molecule design that can be made bearing three amino acid side-chains. Conception of this scaffold corresponds to box 1 in the flow chart FIG. 7. The EKO process is sensitive to the stereochemistries of chiral centers in that scaffold.

Box 2 of FIG. 7 indicates that accessible preferred conformations of the featured scaffold must be determined. These are thermodynamically preferred conformations being within a user defined energy (eg 3 Kcal·mol⁻¹) of the lowest energy conformer identified. For instance, it was necessary to determine the thermodynamically accessible conformations of scaffold 1aaa-H, and classify the conformations based on how they present side-chains (EKO, Exploring Key Orientations, see below, eventually relates these side-chain orientations to ones in PPIs). To do this, we use the Quenched Molecular Dynamics (QMD) technique, but other computational methods to identify preferred conformations could be used.

We routinely determine preferred orientations for the scaffold with three methyl side chains (eg 1aaa-H) because this best represents the intrinsic bias of the scaffold when it bears some side-chain other than hydrogen. Modifications would be envisaged where determination of preferred conformations would be performed for the scaffold with side-chains other than methyl, eg ethyl or CH₂OH. Methyl was chosen because it is the simplest system for which the intrinsic conformational bias of the molecular scaffold can be simulated and, at the same time, the orientations of side-chains than are attached to that scaffold (in the positions represented by the methyl groups).

Box 3 and 4: each preferred conformer of 1aaa-H is represented by six coordinates corresponding to the 3×(Cα−Cβ) atoms.

Conformational analyses with QMD typically generate 600 low energy structures. These conformations are clustered into families with similar RMSDs (0.5 Å is typical) based on Cα and Cβ coordinates. The conformer from each family having the lowest energy is always selected. However, within each family, there are “sub-clusters” of structures having similar RMSD values relative to the lowest RMSD structure in the family. Thus sub-clusters in each family can identified (this is done by the user when he/she sees how the conformers best groups together) and the lowest RMSD from each of these is also earmarked for matching using the algorithm described below.

It might seem that for EKO to work, the QMD experiment should be performed using the side-chains that correspond to each PPI target, and in a special medium; whereas, in fact we set all three side-chains as methyl (Ala-Ala-Ala derivative), and use a featureless medium of dielectric 80 (corresponding to water). Although QMD analyses where the side-chains are not methyl do predict slightly different preferred conformers, this is unimportant for the following reason. The QMD experiments show that 1aaa-H can attain an ideal conformation to present three functionalized side-chains when bound to the protein binding partner. EKO shows which PPI structures have corresponding orientations of the full side-chains. Thus both the scaffold (in a featureless medium) and the protein (in a PPI) favor the same side-chain orientations otherwise EKO would not find them. In other words, the environment provided by the protein binding-partner in the PPI corresponds to a favored orientation of 1aaa-H, and vice versa. Perturbations to the preferred conformations of the scaffold with other side chains in the absence of the protein are inconsequential. Thus in the featured example, modeling and mining was conducted using the compound 1aaa-H, but compounds based on 1 gain affinity and selectivity through the incorporation of side-chains which correspond to the PPI target.

### Typical Algorithm to Implement the EKO Strategy

As shown in Box 5 (FIG. 7), we ran the protein database generated by “3D complex” that covers all protein structures released before 2008, but the same principles can be applied to all pdb files for PPIs in the PDB that identify side-chain locations. The 3D complex database encompasses over 53,000 PPI-interfaces (or “protein chain interactions”) in 15,736 structures. However, similar databases, and the section of the whole PDB that covers PPIs (homo- and hetero-oligomers) can also be used. Not all side-chains are involved at PPI-interfaces, so filters are applied to focus EKO on the most pertinent side-chains. The first filter used in this work is a user defined parameter: for a side-chain to be considered it must be within X Å of the other protein chain; typically, this distance “X” is set at 4 Å (Box 7). Another side-chain filter in the algorithm we used applies to the “angle” made by the side-chain to the other protein chain (Box 8). Interface side-chains remaining after the first filter is applied are only considered if their Cα−Cβ vector points toward any non-H terminal side-chain atom at the PPI interface. Yet another filter that is applied is as follows: triplets of side-chains that pass the other two filters are only considered if they also have their Cα atoms within a user-defined distance of each other, eg 10 Å (Box 9).

The filters described above are applied to restrict the answer set to matches that are truly relevant to perturbation of PPIs, and to keep the volume of data to be processed reasonable so that the process can be applied with minimal computational resources. Other filters may also be added; for instance, we have optionally modified the procedure so that only one set of crystal data is included for each PPI even though many crystal structures on this PPI may be in the PDB. Alternatively, the input database can be made to exclude structures that may be uninteresting (eg ones involving antibodies). Conversely, it is possible to design an algorithm that runs without any or all of these filters, but if less filters are used the answer set will be less relevant and take longer to determine.

The output of the data mining exercise is many sets (potentially hundreds of thousands) of six coordinates (3×Cα−Cβ corresponding to each “triplet” of amino acid side-chains that passed the filters outlined above (Boxes 9 and 10).

The process of overlaying “triplet coordinate sets” from the preferred conformations of the small molecule with the interface side-chain triplets is depicted in Box 11. These combinations are then systematically overlaid with the six coordinates corresponding to the Cα−Cβ atoms of the scaffold (i.e., peptidomimetic compound) side-chains in each of its preferred conformations. A computer program then records the RMSD for each overlay, and moves on to the next structure. The program then ranks the hit interface matches for goodness of fit overlay of conformer side-chains on one protein component at a PPI. In general, smaller the RMSD values indicated better fits. There are numerous overlay routines that can be used for this, and many ways of expressing the goodness of fit, besides RMSD.

After each superposition is scored for goodness of fit, and the output is a prioritized list of PPIs corresponding to a particular PDB, and three particular side-chains at the interface of one protein strand (Box 12). Molecular dynamics of the small molecules is routinely performed using three methyl side-chains, but the Cα−Cβ coordinates of the matching PPI triplets can correspond to any amino acid side-chain. For example, we applied this technique to 368 conformations from compound L,L,L-1aaa-H for 15,736 different crystal structures, and found 106 hits corresponding to conformations of the small molecules that overlay on interface residues with RMSDs of 0.3 Å. Nearly all of these PPI interface regions do not involve three alanine side-chains.

A major attribute of the method described above is the efficiency with which a large number of accessible conformers of a small molecule expressing amino acid side-chains may be compared with orientations of side-chains at a protein-protein interface. This addresses the difficult issue of deciding which protein-protein interfaces have regions that might be expected to be perturbed by the featured small molecule.

The output therefore predicts exactly which side-chains should be presented by the scaffold to perturb the corresponding PPI (Box 13). This predictive tool does not prove that the small molecule will perturb that particular PPI, but instead indicates that the EKO process points to this situation as being statistically favored over others with other side-chains or scaffolds.

The process can be reiterated for many PPIs without human intervention (i.e., computationally) (Box 14).

To screen protein structures of particular interest that may not be in the input database (eg those after Jan. 1, 2008 that are not in the first version of the 3D complex database) we developed a similar algorithm to match conformations on one or more selected crystal structures using the same principles. This can be used to: (i) match any PPI crystal structure, including those released after 2008; (ii) use all the conformations of the molecule without clustering; and, (iii) consume less CPU because only select PPI structures are considered.

Accordingly, provided herein is an algorithm concept for matching protein-protein interactions with one or more preferred conformations of a compound. In one embodiment, the protein-protein interactions contain an interface region having at least three amino acid side-chains. In another embodiment, the orientations of three amino acid side-chains in an interface region of the protein-protein interaction is matched to the Cα and Cβ coordinates of one or more preferred conformations of the compound.

The methods and algorithms described herein can be utilized automatically via a computer. Accordingly, provided herein is a computer-program-concept for instructing a computer to perform the methods disclosed herein. Also provided is a computer program for instructing a computer to perform the methods disclosed herein. Also provided herein is a computer program utilizing the algorithms disclosed herein.

### Part IV. Examples of PPIs Matched to Molecules 1Aaa-H Using the EKO Approach

As described in Example 1, compounds useful for the inhibition of HIV-1 protease by perturbing the PPI interface between the two monomers were predicted using the EKO approach, and validated by preparing the appropriate molecules then testing them for activity as inhibitors of HIV-1 protease. Similarly, compounds useful for the inhibition of several other biological targets have been predicted, as described below in Examples 2-6.

### Example 1

### Dimerization Inhibitors for HIV-1 Protease

### HIV-1 Protease: Hot-Spots and Energetics for HIV-1 Protease Dimerization

HIV-1 protease exists as a stable homodimer for which the Gibbs energy of stabilization has been estimated to be ca 14.5 kcal/mol at 25° C. (pH 5), corresponding to a dissociation constant of 2.3×10⁻¹¹M, or 3.4 nM at 37° C. Isolated subunits of the protease are intrinsically unstable,²⁰ implying that if the dimers are “cracked” the subunits would misfold and be vulnerable to proteolytic degradation. It is therefore pertinent to look carefully at the dimer-dimer interface.

Hot-spots for the PPI between HIV-1 protease monomers appear to be at the C- and N-termini; Cys95-Thr96-Leu97-Asn98-Phe99 and Pro1-Ile3-Leu5; these account for about 75% of the total binding energy (based on Ala-scanning/differential scanning calorimetry). These residues are relatively hydrophobic, as expected for a homodimer interface region. Mutation of Cys95 to Ala has little impact on the protease activity, and presumably on the dimerization energy too. This is fortunate because methyl side-chains can be used in place of —CH₂SH groups so that small molecule mimics are more easily made and manipulated.

Peptides have been designed to perturb the dimerization face of HIV-1 protease. These were based on the C-terminus, the C- and N-termini separately, the C- and N-termini linked by a hydrophobic chain, or C- and N-terminal peptides linked through a side-chain. Efforts to improve cell permeabilities of these compounds have featured linkage of alkyl chains to the peptides (but no cellular activity was reported), or combinations of the peptide with HIV-TAT, but these systems are vulnerable to proteolytic degradation and are delivered into endosomes.

A few peptidomimetics of “dimer-disrupting” peptides for HIV-1 protease also have been prepared. Thus, essential NH residues have been mapped via N-methylation procedures, then completely N-alkylated systems, “peptoids” were made and tested giving compounds with low micromolar IC₅₀ values. A set of Bartlett's @tides gave compounds with K_{d} values of about 400 nM. It has also emerged that some non-peptidic compounds that bind the active site also act as dimerization inhibitors.

Results from Application of the EKO Method

Quenched molecular dynamics (QMD) was used to do simulate preferred conformers of 1aaa. Only conformers within 3 kcal/mol of the most stable one identified were considered. This “3 kcal/mol cut-off” gave the following number of conformers for each stereoisomer of 1: LLL-(490), DLL-(490), LDL-(453), LLD-(512), LDD-(489), DLD-(511), DDL-(487), and DDD-(466).

A data mining algorithm developed “in house” was the used to take each preferred conformation as an input, expresses it as six coordinates {3×(Cα+Cβ)}, and quantify the “goodness of fit” of these on all combinations of three amino acid side-chains in all the structurally characterized PPI interfaces that are entered. Over 53,000 PPI-interfaces corresponding to 15,736 structures was sampled. For eight stereomers of 1aaa, EKO exposed a total of 391 unique PPI-interface regions where orientations of side-chains in preferred conformations matched those at interfaces with RMSDs≦0.30 Å.

The output of this algorithm is a relatively long list of interface regions that matched with preferred conformers of the featured compound. Data from mining a single isomer of 1 takes too much space to show here, but Table 2 illustrates an EKO output for the eleven best “hit” interface overlays, and three others (red), from L,L,L-1aaa. Entries 15, 16, and 23 are, in our view, biomedically significant PPI targets that would interest researchers considering synthesis of molecules with type 1 chemotypes.

TABLE 2
Summary for data mining L,L,L-1aaa

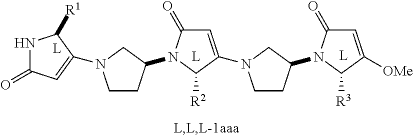

| | | | | residues |
|---|---|---|---|---|
| entry | PDB | proteins | RMSD (Å) | (R¹-R²-R³) |
| 1 | 1kn0 | Rad52 | 0.14 | H121-S119-D117 |
| 2 | 1n2c | nitrogenase | 0.19 | K145-D76-S257 |
| 3 | lg0o | trihydoxynaphtalene | 0.23 | P173-H122-V126 |
| | | reductase | | |
| 4 | 1j3u | aspartase | 0.23 | V236-T234-V232 |
| 5 | 1g17 | TrwB | 0.23 | T352-D349-S346 |
| 6 | 1six | trypsin-ecotin | 0.24 | Me5-T83-L52 |
| 7 | 3pcb | 3,4-PCD^{a} | 0.24 | Q177-175-K173 |
| 8 | 1fcj | O-acetylserine sulfinydrylase | 0.24 | L268-S301-E303 |
| 9 | 2f4f | IS200 transposase | 0.25 | H60-V18-V107 |
| 10 | 1mtp | serpin (thermopin) | 0.26 | Y200-T210-A218 |
| 11 | 1eef | heat-labile enterotoxin | 0.26 | T47-I39-E29 |
| 15 | 1thz | AICAR Tfase^{b} | 0.28 | A218-L220-T222 |
| 16 | 3gpd | GAPDH^{c} | 0.28 | T228-M230-F232 |
| 23 | 1hpv | HIV-1 protease | 0.29 | L97-C95-I93 |

^{a}3,4-PCD: protococatechuate 3,4-dioxygenase.
^{b}AICAR Tfase: avian aminoimidazole-4-carboxamide ribonucleotide transformylase.
^{c}GAPDH: D-glyceraldehyde 3-phosphate dehydrogenase.

In the procedure above, preferred conformations of the featured scaffold are calculated using truncated (Me-) side-chains, but they are overlaid on Cα and Cβ coordinates corresponding to combinations of particular interface amino acids. By making this comparison, EKO searches for intrinsic conformational biases of the scaffold with methyl side-chains that will be reinforced when the molecule binds a protein-binding partner in a hit PPI. Synergy occurs in these situations because the favored scaffold Cα−Cβ orientations coincide with the ways the rest of these side-chains are bound by the protein binding partner at the PPI-interface.

EKO side-steps the most problematic issues encountered in simulations of small molecules interacting with protein surfaces by focusing on static interface regions in structurally characterized PPIs. Structural data clearly shows the interface regions and the side-chain orientations circumventing the issue of how the small molecule and protein might flex to adapt to each other. EKO determines situations where the structurally characterized PPI and favored conformations of the small molecule have similar side-chain orientations: if there are no anomalies in the structural data then those side-chains are sterically and physiochemically matched.

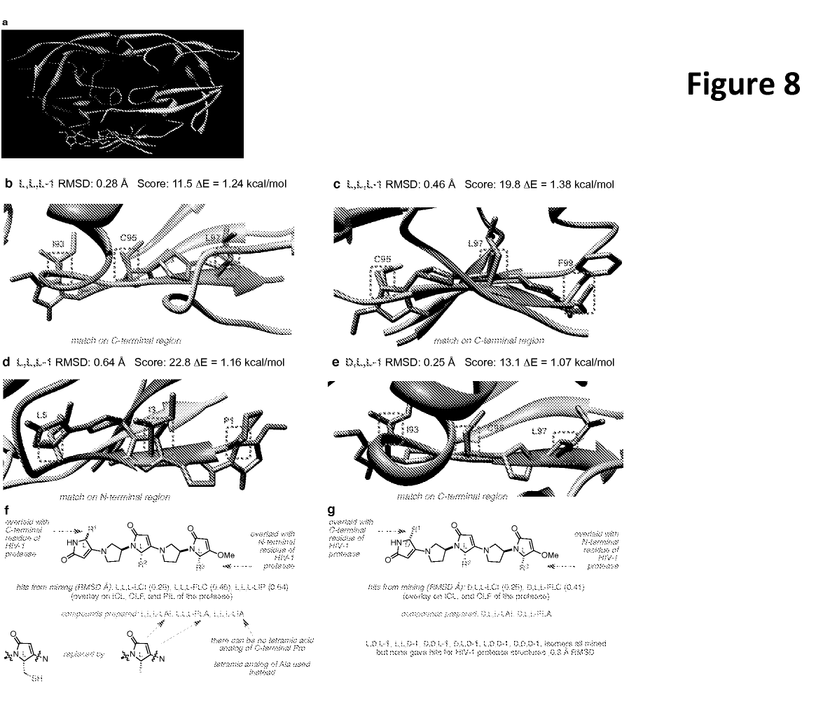

Mining of the 3D complex database for L,L,L-1 showed overlay (RMSD 0.28 Å) on HIV-1 protease interface residues Ile93, Cys95, Leu97 (entry 23 in Table 2; hpv, and in five other HIV-1 protease/ligand or/metal structures; RMSD<0.33 Å). These correspond to the region of the HIV-protease dimer where the C- and N-termini interact to form a four-strand sheet network (FIG. 8a).

Discovery of the hit at 0.28 Å RMSD motivated us to consider the overlays with less exact RMSDs (up to 0.65 Å). This revealed that the protease residues Cys95, Leu97, Phe99 overlapped with a conformer of L,L,L-1 (RMSD 0.46 Å); these are slightly displaced towards the C-terminus relative to the original match (which was Ile93, Cys95, Leu97), and they correspond exactly to the putative hot-spot region.

Both the matches identified above Ile93, Cys95, Leu97 and Cys95, Leu97, Phe99 correspond to C-terminal regions of HIV-1 protease, but we found N-terminal matches. Specifically, preferred conformers of the template overlaid with Pro1, Ile3, Leu5 (RMSD 0.64 Å; FIG. 8d). All these three residues are implicated in a hot-spot region.

Template 1 overlaid with a reverse polarity on the HIV-1 protease fragment, ie the N-terminus of the mimic superimposed with the C-terminus of the enzyme (FIG. 80. This trend persisted when all the stereoisomers of 1 were mined (FIG. 8g; results only for overlays on HIV-1 protease structures considered). Of the other 7 stereoisomers, preferred conformations of only D,L,L-1 were found to overlay with RMSD<0.3 Å (on 17 different HIV-1 protease structures). All these overlays had reversed polarities relative to the HIV-protease chain on which they were overlaid.

The cysteine side-chain is not a convenient one to include in small molecules. Fortunately, as noted above, HIV-1 protease mutants wherein Cys95 was replaced with Ala have almost the same K_{d} for the dimer dissociation; consequently, our primary target is LAI rather than LCI.

### Primary Assays (Enzyme Kinetics)

A widely accepted strategy for assessing in vitro activities of HIV-1 protease dimer-disrupting compounds has emerged from the literature in this area. First, dimer disruption is monitored via a fluorescence-based assay involving cleavage of a peptide with quencher and fluorescent groups at either termini. After that, an enzyme kinetic analysis via the Zhang-Poorman method is performed.

### Example 2

### AICAR Tfase Inhibitors

### AICAR Tfase and Cancer

5-Aminoimidazole-4-carboxamide ribonucleotide transformylase (AICAR Tfase) is one component of a bifunctional enzyme, the other being inosine 5′-monophosphate cyclohydrolase (IMPCH). These catalyze the last two steps in purine biosynthesis. Formyl transfer from the cofactor 10-formyl-tetrahydrofolate (10-f-THF) to the aminoimidazole functionality is mediated by AICAR Tfase, then IMPCH promotes cyclization of this N-formyl group to give the purine framework (of IMP).

Normal cells generate most of the purine they require by a salvage pathway; for them, de novo biosynthesis is relatively unimportant. However, cancer cells depend heavily on the de novo pathway, hence they are vulnerable to inhibitors.

AICAR Tfase is one of several folate-dependent enzyme targets for chemotherapy {cf thymidylidyl synthase, dihydrofolate reductase, and glycinamide ribonucleotide transformylase}. Inhibitors of AICAR Tfase that are not based on folate have advantages over other anti-folate drugs (cf DHFR, “DDATHF” {lometrexol} and LY231514) because they are unlikely to impact non-targeted folate-dependent enzymes giving unpredictable side effects. Two validated strategies for disabling AICAR Tfase that do not involve mimicry of folate are: (i) disruption of the active site function; and, (ii) perturbation the interface in the dimer. AICAR Tfase is only active in the dimeric form, and some molecules that disrupt the dimer interface are known to inhibit the enzyme. These molecules are cyclic peptides (Kᵢ 17 μM or more), or flexible small molecules (from HTS, egKᵢ 17 μM). Thus the only approaches used so far to disrupt AICAR Tfase dimerization have been combinatorial, involving large numbers of randomly produced compounds; they give relatively weak inhibition.

Results from Mining

Hits for screening L,L,L-1 had the sequence ALT; these align with a sheet region on the interface (RMSD 0.28 and 0.31; Table 3). Relaxing the RMSD requirement gave two more hits (0.39 and 0.42;). Mining all the amino acid-based stereoisomers of 1 gave only one more hit, for L,D,L-1, and this matched a different region of the protein L329-E331-K333. All the mimics aligned parallel with the strand.

TABLE 3
Matching of stereoisomers of 1 on AICAR structures.

| | | | RMSD | | | | |
|---|---|---|---|---|---|---|---|
| | conformer | PDB | (Å) | score | residues | direction | source |
| 1 | LLL | 1thz | 0.28 | 17.6 | A218-L220-T222 | N -> C | chicken |
| 2 | LLL | 1m9n | 0.31 | 19.3 | A238-L240-T242 | N -> C | chicken |
| 3 | LDL | 1zcz | 0.26 | 17.0 | L329-E331-K333 | N -> C | thermotoga maritima |

### Example 3

### Cholera- and Entero-Toxin Interface Inhibitors

These toxins are directly associated with cholera and related enteropathies in humans and domestic animals. Diarrhea is perhaps the leading worldwide cause of mortality for children under 5, and the featured toxins are responsible for a significant fraction of these deaths.

Both toxins consist of a 27 kDa A fragment which sits on top of a cyclic homopentamer of 11.7 kDa B fragments giving an AB₅ quaternary arrangement. Over 80% of both the A and B fragments in the two toxins share the same amino acid sequence. Template 1 overlaid with E. coli enterotoxin from pdb:1eef. Specifically, L,L,L-1 gave a good overlay on the highly discontiguous residues T47, 139, E29. Relaxing the RMSD requirement exposed two other matches d and e at Y27, E29, M31 and A98, S100, K102.

In the disease progression, bacterial cells express the constituent A and B fragments, and these assemble into the AB₅ hexamer units. It is the B₅ units of the AB₅ structures that bind the ganglioside GM1 receptor of the host's epithelial cells. Binding of the B₅ pentamer triggers down-regulation of pro-inflammatory immune responses. Receptor-mediated endocytosis delivers the toxin into the cells, then the A unit is proteolytically cleaved. This fragment catalyzes ADP ribosylation of the Gαₛ subunit of the heterotrimeric G protein resulting in constitutive cAMP production, secretion of water and salts into the lumen of the small intestine resulting in rapid dehydration and other factors associated with cholera.

Though there has been no thermodynamic work to determine hot-spots, each B-unit in the B₅ structure shares an extended protein-protein interface, making the pentamers extremely stable. They maintain their secondary structures in ionic detergents, 8 M urea, 7 M guanadinium hydrochloride, and to temperatures>80° C. in aqueous solution. Thus, a high activation energy (151 kJ/mol) has been measured for disassembly on the pentamer units, but they can be denatured into monomeric fragments at pH 2 or less. Dissociated toxins formed at low pH assemble at experimentally convenient rates once the medium is made neutral again.

Small molecule interface mimics possibly will not cleave the preformed toxins B₅ pentamers since they are so stable. However, the impact of the mimics could be assayed in vitro by monitoring their effect on their rate of re-assembly after pH reduction, then restoration to neutrality. A possible therapeutic mode of action for compounds that suppress assembly of the B₅ units would be via penetration of the small molecule into bacterial cells preventing expression and formation of the mature hexamer before it is released.

Data from Mining

Mining L,L,L-1 gave a hit (RMSD 0.26 Å) which overlaid on T47-I39-E29, and relaxing the RMSD requirement gave M31-E29-Y27 (0.42 Å) and K102-S100-A98 (0.49 Å). MEY corresponds to a region of the interface that is known to be vital for H-bonding.

Structures of the featured toxins were mined for all 8 amino acid-based epimers of 1. Ten hits were observed for L,L,L-1, and all of them matched on the same protein region T47, 139, E29. Only L,D,L-1 of the other isomers matched, and this time with I31, E29, and Y27 of the cholera toxin, which corresponds to the “second tier” hit (M31, E29, and Y27) for matching L,L,L-1 on the enterotoxin (5 hits); all the matches were C-to-N.

### Example 4

### α-Antithrombin

### Relevance to Neurological Diseases: Serpins and Serpinopathies

Serpins are serine protease inhibitors that are active in their monomeric forms, but can revert to inactive fibril-like oligomers. Formation of these fibrils is an undesirable characteristic associated with a series of diseases collectively known as “serpinopathies”.⁶¹ Serpinopathies are driven by conformational changes to proteins that lead to fibrils, in ways that parallel, but are different to, amyloid formation in Alzheimer's disease. Overall, interaction of one serpin unit with another, a PPI, governs these events.

A key feature of serpin oligomerizations is that the monomeric proteins are metastable; they revert to thermodynamically more favorable (ca 32 kcal/mol) dimeric, then oligomeric forms via a domain swapping process. This involves opening of the proteins via release of a loop region that is intimately associated with a β-strand arrangement in the monomeric form. There is apparently a significant kinetic barrier to formation of the dimeric form, but once this is reached, it opens a gateway to oligomerization. Thus, dimer formation in serpinopathies has been described to impart “infectivity”. Discovery of a small molecule that can modulate such processes for one serpin would have ramifications for all serpinopathies.

Intriguingly, like Alzheimer's, several serpinopathies are associated with neurological diseases. These include, for instance, involvement of neuroserpin in the formation of “Collins bodies”, a characteristic of familial encephalopathy. However, probably the most studied of the serpins is α-antitrypsin; mutated forms (the “Z-mutant”) of this protein are associated with liver cirrohosis and emphysema. Of particular interest here is another serpin called antithrombin. Mutations of antithrombin are associated with thrombosis, and blood-clotting events in thrombosis are related to stroke.

Various groups have investigated how peptides corresponding to the loop region involved in domain swapping can be used to inhibit oligomer formation in serpinopathies. For instance, this approach has been proven for α-antitrypsin, and antithrombin. In vitro assays used to identify the active peptides in these studies involve differentiation between serpins in monomeric and oligomeric forms. This can be done by gel-electrophoresis and by methods that rely on intrinsic Trp fluorescence.

Ultimately, peptides that are active in vitro are unlikely to be useful in vivo due to the usual reasons associated with bioavailability (cellular and stability to proteases; oral is not required since intravenous injection of therapeutics for life threatening disease is standard and acceptable). Consequently, the awaits for small molecules to be discovered that exert similar inhibition of dimerization properties. This is analogous to the stage of development of Alzheimer's therapies when peptide leads were shown to inhibit amyloid formation.⁷⁰

Results from EKO Mining

Domain swapping processes leading to antithrombin oligomers involves a loop-sheet interaction in the monomeric closed form being transformed into a similar one between one or more protein monomers. Data mining experiments for this proposal were performed using the only available structural information (human antithrombin, 2znh), and that involves the wild type antithromin and not the mutated one that most inclined to form firbils. Hit PPI regions where EKO predicts compounds 1 could bind (see below) correspond to the sheet region where that the loop interacts with in the closed form. Antithrombin mutations that lead to fibril formation are not associated with this loop-sheet interaction and, because of this, the inhibitors that are designed here should be appropriate for mutated serpin.

Table 4 summarizes the interface mimics 1 found after data mining all the stereoisomers; all but one overlaid on the sheet region that is either side of the key loop; the exception (Table 4, entry 5) overlaid on an ill-defined helix-loop motif L,L,L-1aat overlays with the C-terminus of the mimic on the C-terminal end of the featured interface; in other words, the two chains that are overlaid run in the same direction, so we call this an N->C mimic (natural orientation). Overlays for the other mimics listed in Table 1 are superior to this in terms of RMSD.

Table 4 shows that some interface mimics 1 may “align” the protein strand and others “oppose” it (N->C, and C->N respectively) but both can give good side-chain fitting. One compound, D,L,L-1efa overlays with discontiguous amino acids that reverse relative to the mimic (C->N->C).

TABLE 4

| | | RMSD | | | |
|---|---|---|---|---|---|
| | conformer | (Å) | score | residues | Direction |
| 1 | LLL | 0.37 | 15.2 | A382-A384-T386 | N -> C |
| 2 | DLL | 0.28 | 17.6 | E374-F372-A384 | C -> N -> C |
| 3 | LDL | 0.25 | 15.1 | S385-H369-A367 | C -> N |
| 4 | LLD | 0.34 | 21.8 | L373-A383-S385 | N -> C |
| 5 | DDL | 0.36 | 19.5 | D97-C95-A20 | C -> N |
| 6 | DLD | 0.33 | 17.0 | V388-T386-K370 | C -> N |
| 7 | LDD | 0.23 | 12.6 | A384-A382- E374 | C -> N |
| 8 | DDD | 0.34 | 14.5 | H369-A387-V389 | C -> N |

### Example 5

### D-Glyceraldehyde-3-phosphate Dehydrogenase

### Relevance to Neurological Diseases

D-GlycerAldehyde-3-Phosphate DeHydrogenase (GAPDH) mediates oxidative phosphorylation of the aldehyde after which it is named; this is a key step in the glycolytic pathway.⁷¹ The structure of GAPDH is a homotetramer or, more accurately, a dimer of dimers, wherein the active site is a NAD⁺ binding groove found on each monomer component.

GAPDH is implicated in apoptotic cell death, particularly in neurodegeneration. Thus, in cellular assays, rescue from apoptosis can be affected by antisense suppression of GAPDH or using the Parkinson's therapy (R)-deprenyl (Selegiline). Further, a tricyclic deprenyl analog, CGP3466, binds and stabilizes the dimeric form of GAPDH and has 100× the rescuing effect of deprenyl in vitro; CGP3466 is a neuroprotective drug that has featured in clinical trials for Parkinson's disease and ALS. Consistent with these observations, certain fractions of cerebrospinal fluid (CSF) from Parkinson's patients cause apoptosis when added to cells in culture, whereas CSF from healthy patients does not. Further, the apoptidic effects of CSF from Parkinson's patients is prevented by antisense targeting of GAPDH or by (R)-deprenyl.

Based on the assertions above, nefarious roles of GAPDH in several neurodegenerative diseases are implicated. Exact mechanisms that tie GAPDH to apoptosis in neurological diseases like Huntington's, Parkinson's, Alzheimer's, ALS, stroke and glaucoma (neurodegeneration of retinal ganglion cells) are not known, but this has been an area of intense recent interest (GAPDH in human neurodegenerative diseases has been reviewed) and some clues are emerging.

GAPDH has to be imported into the nucleus to trigger apoptosis. After this, nuclear accumulation of GAPDH, or an isoform of it, occurs in the neurological diseases mentioned so far. Association of cystolic GAPDH with the E3 ubiquitin ligase Siah 1 is critical for importing the former into the nucleus, because only the latter has a nuclear localization signal. It has been proposed that CGP3466 may bind the NAD⁺ site causing structural changes that reduce the affinity of GAPDH for Siah 1; in other words, the drug inhibits apoptotic activity of GAPDH by preventing its nuclear localization. Precisely what form of nuclear GAPDH triggers apoptosis is unclear; some evidence suggests that GAPDH-complexation stabilizes the otherwise short-lived Siah 1, but another explanation is that activation of transcription induced by nuclear GAPDH initiates apoptotic cell death via a network of signaling mechanisms. Once inside the nucleus, there appears to be a change in GAPDH structure associated with oxidative modification of a channel Cys residue (#149 or 150 depending on the species). It has been suggested that this modification might be a signal for transcriptional activation of its own gene, but there is no evidence for this at present.

GAPDH binds to unusual oligopeptides that are found in neurodegenerative diseases, but the relevance of this is unclear. Polyglutamine-repeat regions localized in cell nuclei correlate to disease progression and severity in several neurological conditions. Some proteins are known to selectively bind (Gln)ₙ strands, and one of those is GAPDH. Consequently, even though the neurological effects of GAPDH/(Gln)ₙ accumulation in cell nuclei are currently unknown, there is an open possibility that this may have causative deleterious effects. Similarly, in Alzheimer's disease, GAPDH binds the cytoplasmic carboxyl terminus of the b-amyloid protein, and the significance of this is also unresolved.

Overall, there are many possibilities for ways in which GAPDH could be perturbed in therapeutic approaches, particularly in view of the unknowns surrounding its role in the onset and progression of neurological diseases. Our hypothesis is that the quaternary structure of GAPDH may influence the role of this protein in programmed apoptosis, impacting accumulation of the enzyme in the nucleus and what it does there. We are intrigued by the observation that the dimeric forms do not induce apoptotic activities, even though they are more active in glycolysis because this supports our supposition that interface mimics to perturb the dimerization state of GAPDH may selectively effects apoptosis in neurodegeneration (cf, when CGP3466 binds rabbit GAPDH in vitro, it converts the tetramer to a dimeric form, and that is more active than the parent tetramer in glycolysis). The fact that CGP3466 gives 100× the apoptotic rescuing effect of deprenyl may be because it changes the enzyme to the dimeric form more effectively via a different allosteric binding mode. In other words, perhaps deprenyl has less influence on the interface region than CGP3466, converts it to the dimeric form less effectively, and gives less of an apoptotic rescuing effect. Our preliminary studies have uncovered an opportunity to prepare small molecule interface mimics to perturb assembly and persistence of GAPDH monomers into dimers-of-dimers, and we propose to test compounds that are designed to disrupt these interface regions.

Results from Mining

There are two types of interfaces in the GAPDH tetramer; one composed of mainly sheet regions (dimer interface) accounts for a large area of interfacial overlap, and another where the interface is a less well-defined loop (dimer of dimer interface). Overlay of template 1 on the loop region gave unsatisfactory RMSD values, but fit of template 1, and the stereoisomers of this, on the other interface gave some excellent matches.

The core molecule L,L,L-1 overlaid on the sheet interface region of human GAPDH (pdb: 3gpd in which NAD⁺ is also bound) with an RMSD of 0.28; specifically, it matched in a parallel fashion (ie N-termini of protein and peptidomimetic are head-to-head) with residues F232, M230, and T228 on a b-sheet at the hydrophobic interface formed with a b-sheet on the other monomer.

Overlays of the same stereoisomer L,L,L-1aaa-H on the same crystal structure but at slightly higher RMS deviations gave a second hit, interesting in three respects: (i) it overlaid at a different part of the sheet interface region; (ii) the side-chains involved were on discontiguous residues (K308, 65 residues displaced from D243, and V241); and, (iii) the compound rests antiparallel with the primary sequence (C->N).

In the next phase of the mining exercise we applied the EKO process all the other stereoisomers (D,L,L-, L,D,L-, L,L,D-, D,D,L-, D,L,D-, L,D,D-, and D,D,D-) of 1aaa-H on all the available GAPDH structures, and many leads emerged. Molecules 1 overlaid N->C or C->N with the protein. Interestingly, L,D,L-1 gave more matches than any other isomer, and these could be aligned or inverse oriented. Before synthesis of the compounds, we will check the sequence correspondence between organisms for each of the three amino acid combinations outlined in Table 5, and match them to the source of GAPDH used in the assay (human is preferred; GAPDH is commercially available for all the organisms listed below, rabbit is one of the least expensive, and human is the most).

TABLE 5

| | | RMSD | | | | |
|---|---|---|---|---|---|---|
| conformer | PDB | (Å) | score | residues | directionality | source |
| LLL | 3gpd | 0.28 | 12.30 | F232-M230-T228 | N -> C | human |
| LLL | 1j0x | 0.32 | 16.80 | F230-M228-T226 | N -> C | rabbit |
| DLL | 1gd1 | 0.30 | 18.69 | D242-V244-E246 | N -> C | bacillus stearothermophilus |
| LDL | 2hki | 0.24 | 14.41 | K309-I311-W313 | N -> C | spinach |
| LDL | 1znq | 0.26 | 13.40 | L177-T179-V181 | N -> C | human |
| LDL | 1cer | 0.25 | 12.55 | F306-K304-M302 | C -> N | thermus aquaticus |
| LDL | 1dc3 | 0.27 | 12.54 | L171-T173-V175 | N -> C | E-coli |
| LDL | 1qxs | 0.27 | 13.19 | L189-T191-I193 | N -> C | trypanosoma cruzi |
| LDL | 1ml3 | 0.29 | 12.54 | L189-T191-I193 | N -> C | trypanosoma cruzi |
| LDL | 2b4t | 0.29 | 12.55 | L183-T185-V187 | N -> C | plasmodium falciparum |
| LDL | 1i33 | 0.29 | 13.38 | L189-T191-I193 | N -> C | leishmania mexicana |
| LDL | 1rm3 | 0.29 | 13.98 | W313-I311-K309 | C -> N | spinach |
| LDL | 2prk | 0.3 | 13.39 | W313-I311-K309 | C -> N | engyodontium album |
| LDL | 1j0x | 0.3 | 17.71 | W310-I308-K306 | C -> N | rabbit |
| LLD | 1nqa | 0.12 | 7.74 | M231-T175-M173 | C -> N | bacillus stearothermophilus |
| LLD | 2dbv | 0.14 | 8.09 | M231-T175-M173 | C -> N | bacillus stearothermophilus |
| DDL | 1cf2 | 0.29 | 12.85 | T215-V213-I183 | N -> C | methanothermus fervidus |

### Example 6

### Caspases 1 and 3

### Relevance to Neurological Diseases

Caspases (cysteinyl aspartate-specific proteases) are intracellular enzymes that specifically cleave substrates at Asp-residues. Intracellular modulation of caspases is achieved, in the first instance, by activator (eg APAF-1, Fas/FADD) and inhibitor (IAP) proteins. At a second level, the activators are controlled by Bcl-2 family and SMAC proteins which modulate the inhibitors. Above that level are Bcl-2 family modulators like Bim, Bad, and Bid). Thus, Nature uses a hierarchical set of PPIs to control caspase activities in cells.

Eight of the eleven caspases encoded by the human genome function in apoptosis. Two processes turn on caspases: (i) extrinsic pathways spurred by activation of cell surface death receptors and mediated by activation of a caspase zymogen by an “up-stream” caspase (eg caspase 8); or, (ii) intrinsic pathways originating in the mitochondria for which caspase 9 is a typical upstream activator. Signals from both the intrinsic and extrinsic pathways converge at downstream caspases like 3 making this a fundamentally important target for control of apoptosis in neurodegeneration (and cancer).

Three human caspases activate a subset of proinflammatory cytokines, and these include caspase 1 (or “interleukin-1β-converting enzyme, ICE”). Selective inhibition of caspase 1 prevents production of IL-1β at sites of inflammation. Activation of caspase 1, on the other hand, causes mature IL-1β to bind to its type 1 receptor and this plays an important role in promoting neuronal cell death.

Selective inhibition of caspase 1 or 3 could have a range of biochemical consequences. These are difficult to predict, but evidence that enhanced caspase 1 and 3 activity is associated with many adverse neurological conditions (reviewed several times) is indisputable. Ischemic or traumatic injury causes upregulation of caspase 1 and 3 and this has been associated with cell death and neurological deterioration. In Huntington's disease, the protein huntingtin is cleaved by caspase 1 and 3 to afford toxic fragments required for the formation of neural intranuclear inclusions and progression of the disease. Inhibition of caspase 1 slows progression of Huntington's disease in a mouse model. Caspase 1 is also implicated in stroke, ALS, and Parkinson's disease. Caspase 3 is pivotal in apoptotic death in Parkinson's disease, ALS, and Huntington's disease; it also regulates neurogenesis and synaptic activity. In Alzheimer's, caspase 3 (and others) cleave the β-amyloid precursor protein (β-APP) giving a C-terminal fragment that is found in senile plaques and is a potent inducer of apoptosis. For these reasons there has been a great deal of interest in small molecule inhibitors of caspases. Nearly all of the known inhibitors were made to target the enzyme active site and not PPIs that retain the enzyme structure.

Structures of Caspases 1 and 3, and Results from Mining

Caspases 1 and 3 have “dimers of heterodimers” quaternary structures wherein each heterodimer consists of a small and larger fragment (p10 and p20 for caspase 1, and p12 and p17 for caspase 3). Active sites are formed at each small/large interface, hence there are two in the overall quaternary structures. The active site His²³⁷ and Cys²⁸⁵ residues are on the larger fragments, and the substrate-binding cavity is completed by the protein-protein interface between this and the small fragment. Consequently, both caspases are obligatory dimers of heterodimers, because dissociation of the small and large fragments negates the activity of the enzyme. A natural inhibitor of caspase 1, the serpin “crmA” acts by opening the p10/p20 interface, and possibly that between p10 and p10 too. We propose small molecule disruptors of PPIs in caspases 1 and 3 could be used similarly to modulate their activities.

Wells and co-workers used their tethering strategy to identify an allosteric site that is found in caspases. This is about 15 Å from the substrate-binding cavity, and it impacts both the p10/p10 and the p10/p20 interfaces without causing dissociation, but trapping the enzyme in an inactive conformation. In human caspase 1 that allosteric site is Arg²⁸⁶, Cys³³¹ (used for tethering), and Glu³⁹⁰. This is particularly relevant here because the compounds that we find may disrupt interfaces in caspase 1 at sites near the ones Wells found amenable to allosteric binding.

Several small molecules were found that impact the p10/p20 interface. In fact, all the 8 stereomers of 1 considered gave good overlays (RMSD<0.22-0.46 Å) with slightly different, but overlapping, amino acid tracts in this region. The fact that several parts of this sheet are involved enhances the scope for small molecule interface mimic design. For instance, using the L,D,L-1 framework, residues N337-S339-R341 overlaid on a loop region of the p10 units (identified in 4 different structures: 1rwk, 1rwn, 1rww, 1bmq). This region is near the active site of the p20 unit, but not directly impacting it. Two matches at slightly higher RMSD were also found; these overlaid with overlapping sheet regions of the p10 unit, T388-F330-I328 (C-to-N) and I328-F330-S332 (N-to-C) that interacts with a sheet on the p20 fragment. One of these matches alternate residues (T388-F330-I328) bridging two strands, whereas the other (I328-F330-5332) is aligned with only one. This type of variance validates our concept of “universal mimic” design.

All the stereomers of 1 also gave good overlays for the p10/p10 interface (RMSD 0.27-0.42 Å). Thus, L,D,L-1 overlaid with E390-T388-M386 (C-to-N) close to the glutamate residue identified by Wells as binding their allosteric inhibitor.

EKO indicated a good overlay of D,D,L-1 on the A227-Y274-E272 sequence in a N-to-C-to-N orientation at the p17/p12 interface. Again, remarkably, all the stereoisomers of 1 gave good overlays with caspase 3 at this interface (RMSD 0.29-0.42 Å). Similarly, matches could be found for the p12/p12 interface but the RMSD values were higher (0.33-0.50) indicating slightly inferior correspondence.

### Example 7

### Results from EKO-Based Data Mining Using Structures 15

The data mining was performed for LLL-14aaa was performed according to the procedure described above to give the data shown in Table 6.

TABLE 6
Crystal structures containing PPIs that the EKO approach
indicates may be disrupted with compounds 14.

| | | | RMSD | residues | secondary |
|---|---|---|---|---|---|
| entry | PDB | proteins | (Å) | (R¹-R²-R³) | structure |
| 1 | 1h4s | prolyl-tRNA synthetase | 0.21 | N65-Y67-L70 | |
| 2 | 2ghr | homoserine o- | 0.24 | D32-E29-E26 | |
| | | succinyltransferase | | | |
| 3 | 1rj7_l | EDA-A1 (Ectodysplasin A) | 0.27 | A121-V123-T72 | β-sheet |
| 4 | 1gyb_1 | N77Y point mutant of | 0.28 | T85-M83-Q72 | β-sheet |
| | | YNTF2 | | | |
| 5 | 1hwk | human HMG-CoA reductase | 0.28 | P513-L509-E505 | helix |
| 6 | 1jsw | L-aspartate ammonia lyase | 0.29 | T348-I344-V339 | helix |
| 7 | 1wyt | glycine decarboxylase | 0.31 | V78-E422-D425 | |
| 8 | 1fpy | glutamine synthetase | 0.31 | 138I-149V-147S | β-sheet |
| 9 | 2cw0_1 | RNA polymerase II | 0.32 | E154-D168-V170 | |
| 10 | 1ryd | glucose-fructose | 0.32 | L264-G262-N242 | β-sheet |
| | | oxidoreductase | | | |
| 11 | 1yg2 | Vibrio cholerae virulence | 0.32 | N157-T153-R148 | helix |
| | | activator AphA | | | |
| 12 | 1o2b | thymidylate synthase | 0.33 | S95-E97-I100 | |
| | | complementing protein | | | |
| 13 | 1jb5 | NTF2 M118E mutant | 0.33 | I82-M102-R120 | β-sheet |
| 14 | 1gzt | type II dehydroquinase | 0.33 | V55-T18-F20 | β-sheet |
| 15 | 1r8x | glycine N-methyltransferase | 0.33 | W117-A86-K89 | |
| 16 | 1hql | The xenograft antigen in | 0.33 | E74-Y72-N238 | β-sheet |
| | | complex with the B4 | | | |
| | | isolectin | | | |
| 17 | 1g7y_3 | 58KB vegetative lectin | 0.33 | E163-S70-A230 | β-sheet |
| 18 | 2p9c | phosphoglycerate | 0.33 | A143-A170-R150 | |
| | | dehydrogenase | | | |
| 19 | 2iy5 | phenylalanyl-tRNA | 0.33 | E509-Y511-S514 | |
| | | synthetase | | | |
| 20 | 1jz6 | beta-galactosidase | 0.33 | D869-E871-S874 | |
| | | human branched chain | | | |
| 21 | 1ekp | amino acid aminotransferase | 0.34 | Y70-Q73-V145 | |
| | | (Mitochondrial)(BCAT) | | | |
| 22 | 1bdy | C₂ domain from protein | 0.34 | E13-L115-E103 | β-sheet |
| | | kinase c delta | | | |
| 23 | 1oxo | aspartate aminotransferase | 0.34 | G110-V103-A257 | |
| 24 | 1vlr | mRNA decapping enzyme | 0.34 | V63-I73-K91 | β-sheet |
| 25 | 1mov | coral protein mutant | 0.34 | F156-N154-F145 | β-sheet |
| 26 | 1tjo | DpsA | 0.34 | H168-D173-V176 | |
| 27 | 2h4u | thioesterase superfamily | 0.35 | F120-R138-T94 | β-sheet |
| | | member 2 | | | |
| 28 | 2f6i | ClpP protease catalytic | 0.35 | N128-F104-T45 | |
| | | domain | | | |
| 29 | 1tar | crystalline mitochondrial | 0.35 | R113-I106-E265 | |
| | | aspartate aminotransferase | | | |
| 30 | 1ls3 | serine | 0.35 | H255-A56-L480 | |
| | | hydroxymethyltransferase | | | |
| 31 | 1rin | lectin-trimannoside complex | 0.35 | I137-I139-V116 | β-sheet |
| 32 | 1g7k | DSRED | 0.35 | H171-E169-Y160 | β-sheet |

1-36. (canceled)

37. A method for identifying a peptidomimetic compound that will perturb an interface region of a structurally characterized protein-protein interaction,
wherein the interface region comprises a sequence of amino acids; and
wherein the peptidomimetic compound comprises an organic scaffold bearing two or more amino acid side chains; the method comprising the steps of:
(i) identifying one or more thermodynamically-preferred conformations of a template molecule, wherein the template molecule comprises the organic scaffold bearing two or more amino acid side-chains, wherein each amino acid side chain is methyl, and wherein the thermodynamically-preferred conformations have energies within 3 kcal/mol of the lowest energy conformation of the template molecule identified via molecular simulations;
(ii) assigning three-dimensional coordinates to the Cα and Cβ atoms of the amino acid side chains in each conformation of the template molecule;
(iii) assigning three-dimensional coordinates to the Cα and Cβ atoms of the amino acid side chains of the interface region;
(iv) overlaying the coordinates from (ii) onto the coordinates of any two or more amino acid side chains from (iii) and selecting an overlay having a goodness-of-fit within a predetermined tolerance; and
(v) identifying the peptidomimetic compound, wherein the amino acid side chains of the compound correspond to the amino acid side chains of the interface region of the overlay selected in step (iv).

38. The method of claim 37, wherein the predetermined tolerance is RMSD<0.7 Å.

39. The method of claim 38, wherein the predetermined tolerance is RMSD 0.2-0.5.

40. The method of claim 37, wherein a computer algorithm is used for steps (i), (ii), (iii), (iv), and/or (v).

41. The method of claim 37, wherein in step (iii), crystallographic data and/or NMR data is used to assign three-dimensional coordinates to the Cα and Cβ atoms of the amino acid side chains of the interface region.

42. The method of claim 37, wherein the peptidomimetic compound has the structure of formula (I): 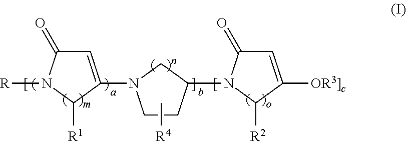
wherein:
R is selected from the group consisting of hydrogen, alkyl, heteroalkyl, and a nitrogen protecting group;
R¹ and R² are independently selected from the group consisting of hydrogen, alkyl, cycloalkyl, heteroalkyl, hetercycloalkyl, aryl, heteroaryl, alkyl-aryl, alkyl-heteroaryl and alkyl-heterocycloalkyl; wherein each R¹ and each R² is optionally, independently substituted one or more times with substituents selected from oxo, carboxyl, carboxamide, carboxyalkyl, hydroxyl, alkoxy, amino, aminoalkyl, thio, thioalkyl and seleno;
R³ is selected from the group consisting of hydrogen, alkyl, heteroalkyl and an oxygen protecting group;
R⁴ is selected from the group consisting of hydrogen, alkyl, alkoxy, aryl and heteroaryl;
Each m is independently 1-2;
Each n is independently 0-2;
Each o is independently 1-2;
a is 0-1;
b is 1-3; and
c is 0-1;
wherein, when b is greater than 1, each R¹ is independently selected from the group consisting of hydrogen, alkyl, cycloalkyl, heteroalkyl, hetercycloalkyl, aryl, heteroaryl, alkyl-aryl, alkyl-heteroaryl and alkyl-heterocycloalkyl, each R⁴ is independently selected from the group consisting of hydrogen, alkyl, alkoxy, aryl and heteroaryl, and each n is independently 0-2.

43. The method of claim 43, wherein the peptidomimetic compound and the template molecule both comprise an organic scaffold bearing three amino acid side chains.

44. The method of claim 43, wherein the peptidomimetic compound is compound 1lai-H:

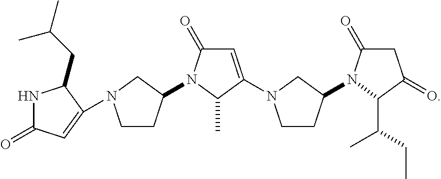

45. The method of claim 37, wherein the template molecule is compound 1aaa-H:

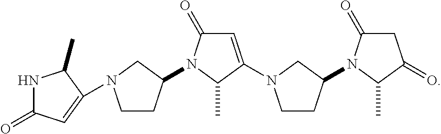 [SMILES: C[C@@H]1NC(=O)C=C1N1CC[C@H](N2C(=O)C=C(N3CC[C@H](N4C(=O)CC(=O)[C@@H]4C)C3)[C@@H]2C)C1]

46. The method of claim 37, further comprising:
(vi) synthesizing the molecule identified in step (v).

47. The method of claim 46, further comprising:
(vii) testing the ability of the peptidomimetic compound to inhibit a protein-protein interaction using an in vitro assay.
